(19) United States
(12) Patent Application Publication
(10) Pub. No.: US 2024/0180912 A1
(43) Pub. Date: Jun. 6, 2024

(54) COMPOSITIONS AND METHODS FOR INHIBITING CREATINE TRANSPORT AS A TREATMENT FOR CANCER

(71) Applicant: Inspirna, Inc., New York, NY (US)

(72) Inventors: Masoud TAVAZOIE, New York, NY (US); Isabel KURTH, New York, NY (US); David DARST, New York, NY (US)

(73) Assignee: Inspirna, Inc., New York, NY (US)

(21) Appl. No.: 18/417,491

(22) Filed: Jan. 19, 2024

Related U.S. Application Data
(63) Continuation of application No. PCT/US2022/073884, filed on Jul. 19, 2022.
(60) Provisional application No. 63/223,926, filed on Jul. 20, 2021.

(51) Int. Cl. A61K 31/519; A61K 31/197; A61K 31/282; A61K 31/513; A61K 31/5383; A61P 35/00

(52) U.S. Cl. CPC A61K 31/519; A61K 31/197; A61K 31/282; A61K 31/513; A61K 31/5383; A61P 35/00

(57) ABSTRACT

The disclosure features compositions and methods that are useful for the treatment of cancer (e.g., gastrointestinal, colorectal cancers) featuring creatine transporter inhibitors (e.g., β-Guanidinopropionic acid) in combination with a KRAS inhibitor or another chemotherapeutic agent (e.g., leucovorin (folinic acid-FOL), 5-fluorouracil (F), and irinotecan hydrochloride (IRI), and/or bevacizumab).

### CROSS-REFERENCE TO RELATED APPLICATIONS

This application is a continuation under 35 U.S.C. § 111(a) of PCT International Patent Application No. PCT/US2022/073884, filed Jul. 19, 2022, designating the United States and published in English, which claims priority to and the benefit of U.S. Provisional Application No. 63/223,926, filed Jul. 20, 2021, the entire contents of each of which are incorporated by reference herein.

### SEQUENCE LISTING

The present application contains a Sequence Listing which has been submitted electronically in XML format. The entire contents of the electronic XML Sequence Listing, (Date of creation: Aug. 29, 2022; Size: 13,162 bytes; Name: 202047-010201PCT_SL.xml), is incorporated by reference herein.

### BACKGROUND OF THE INVENTION

Colorectal cancer is the second leading cause of cancer death in men and women in the United States and a major cause of mortality worldwide. In 2020, roughly 148,000 individuals were diagnosed with colorectal cancer and 53,200 died from the disease. Early-stage colorectal cancers are primarily treated by surgical resection. For larger tumors and those that have spread to lymph nodes, 5-fluorouracil based chemotherapy regimens are administered in the post-surgical ‘adjuvant’ setting to reduce the risk of metastatic relapse. For cancers that have spread to distant organs such as the liver—the primary site of metastatic relapse-systemic 5-fluorouracil containing chemotherapy regimens are given. Targeted therapies including antibodies that engage the Epidermal Growth Factor Receptor (EGFR) or Vascular Endothelial Growth Factor (VEGF) can in combination with chemotherapeutics provide modest survival benefits. However, most metastatic patients succumb to their disease, with a meager 5-year survival of 14%. Recently, innovative small molecules that covalently bind and inhibit the Kirsten rat sarcoma (KRAS) G12C oncogenic driver variant, which is present in approximately 4% of colorectal tumors, have elicited clinical responses in patients harboring this mutant allele.

Other approaches include the development of inhibitors targeting down-stream KRAS signaling in RAS-mutant tumors. Limitations of such therapies include the emergence of resistance which can reduce the durability of an objective response, their lack of efficacy against other KRAS mutant alleles present in ˜40% of CRC patients, and the expected lack of efficacy in KRAS wildtype tumors.

Cancers evolve multiple mechanisms to sustain proliferative growth and survival, which is why cancer is one of the leading causes of death worldwide. One such adaptive mechanism employed by cancer cells is altered metabolism, commonly referred to as metabolic rewiring. By altering various metabolic pathways, cancer cells enhance biosynthesis of cellular building blocks required for growth such as nucleotides, amino acids and lipids. Certain metabolites, however, can become limiting during cancer progression-requiring their extracellular import through metabolic transporters. Gastrointestinal tumors, such as colorectal cancer (CRC) and pancreatic cancers are highly hypoxic, as are metastases formed by these cancers. Metabolic rewiring provides a mechanism to support tumor growth in hypoxic environments and allow for tumors to become resistant to chemotherapy. Therefore, additional approaches and combination therapies are needed to tackle the breadth of various cancer types and metabolic rewiring.

### SUMMARY OF THE INVENTION

In one aspect, the invention features a method of inhibiting cancer cell proliferation or survival, the method involving contacting a cancer cell with a creatine transporter inhibitor and a KRAS inhibitor, thereby inhibiting cancer cell proliferation or survival.

In another aspect, the invention features a method of inhibiting cancer cell proliferation or survival, the method involving contacting a cancer cell with R-GPA, 5-fluorouracil and leflunomide, thereby inhibiting cancer cell proliferation or survival.

In yet another aspect, the invention features a method of inhibiting cancer cell proliferation or survival, the method involving contacting a cancer cell with a creatine transporter inhibitor, FOLFOX, FOLFIRI containing leucovorin (folinic acid-FOL), 5-fluorouracil (F), and irinotecan hydrochloride (IRI), and/or bevacizumab, thereby inhibiting cancer cell proliferation or survival.

In another aspect, the invention features a method of treating cancer in a subject, the method involving administering to the subject a creatine transporter inhibitor and a KRAS inhibitor.

In yet another aspect, the invention features a method of treating a subject having a cancer, the method involving administering to the subject R-GPA or a pharmaceutically acceptable salt, 5-fluorouracil and leflunomide.

In yet another aspect, the invention features a method of treating cancer in a subject, the method involving administering to the subject a creatine transporter inhibitor, FOLFOX, FOLFIRI containing leucovorin (folinic acid-FOL), 5-fluorouracil (5-FU), and irinotecan hydrochloride (IRI), and/or bevacizumab.

In yet another aspect, the invention features a method of treating a selected subject, the method involving administering to the subject a creatine transporter inhibitor and a KRAS inhibitor, where the subject is selected as having a cancer characterized by increased levels of creatine and/or creatine kinase B expression relative to a control cancer.

In yet another aspect, the invention features a method of treating a selected subject, the method involving administering to the subject a creatine transporter inhibitor and a KRAS inhibitor, where the subject is selected as having a cancer containing a KRAS, HRAS, or NRAS mutation.

In yet another aspect, the invention features a method for treating a subject having cancer, the method involving administering to the subject β-GPA or a pharmaceutically acceptable salt thereof and FOLinic acid-Fluorouracil-IRInotecan regimen (FOLFIRI), where (3-GPA or its salt form is administered two times daily at a dosage of about 1000 mg-3600 mg.

In another aspect, the invention features a method of inhibiting cancer cell proliferation or survival. The method involves contacting a cancer cell with β-GPA and Adagrasib, thereby inhibiting cancer cell proliferation or survival.

In various embodiments of any of the above aspects or any other aspect of the invention delineated herein, the method further comprises contacting the cancer cell with an EGFR inhibitor.

In various embodiments of any of the above aspects or any other aspect of the invention delineated herein, the cancer cell is contacted with Adagrasib and β-GPA.

In various embodiments of any of the above aspects or any other aspect of the invention delineated herein, the creatine transporter is SLC6A8. In various embodiments of any of the above aspects or any other aspect of the invention delineated herein the creatine transporter inhibitor is selected from those listed in Table 2 (e.g., β-Guanidinopropionic acid, N-methylamidino-N-methylglycine, 1-carboxymethyl-2-imino-hexahydropyrimidine (cyclocreatine), DL-alpha-guanidinopropionic acid, N-methyl-N-amidino-beta-alanine, N-ethyl-N-amidinoglycine, DL-alpha-guanidinobutyric acid, DL-beta-guanidinobutyric acid, gamma-guanidinobutyric acid, and guanidinoacetic acid, or a pharmaceutically acceptable salt thereof).

In one embodiment, the creatine transporter inhibitor is β-Guanidinoproprionic acid (β-GPA) or a pharmaceutically acceptable salt thereof. In various embodiments of any of the above aspects or any other aspect of the invention delineated herein, a KRAS inhibitor that is any one or more of the following Sotorasib, Adagrasib, MRTX1133, RMC-6291, and RMC-6236. In various embodiments of any of the above aspects or any other aspect of the invention delineated herein, the cancer cell is contacted with β-GPA and a KRAS inhibitor that is any one or more of the following Sotorasib, Adagrasib, MRTX1133, RMC-6291, and RMC-6236. In various embodiments of any of the above aspects or any other aspect of the invention delineated herein, the cancer cell is contacted with β-GPA, FOLFIRI, and bevacizumab. In various embodiments of any of the above aspects or any other aspect of the invention delineated herein, the cancer cell is a breast cancer cell, a gastrointestinal cancer cell, colorectal cancer cell, lung cancer, a melanoma cell, or a pancreatic cancer cell. In various embodiments of any of the above aspects or any other aspect of the invention delineated herein, the cancer cell is a non-small cell lung cancer cell. In various embodiments of any of the above aspects or any other aspect of the invention delineated herein, the cancer is a breast cancer, a gastrointestinal cancer, a colorectal cancer, a lung cancer, a melanoma, or a pancreatic cancer. In various embodiments of any of the above aspects or any other aspect of the invention delineated herein, the cancer is a non-small cell lung cancer. In various embodiments of any of the above aspects or any other aspect of the invention delineated herein, the cancer is a colorectal cancer. In various embodiments of any of the above aspects or any other aspect of the invention delineated herein, the cell is a colorectal cancer cell. In various embodiments of any of the above aspects or any other aspect of the invention delineated herein, the cancer cell contains a KRAS, HRAS, or NRAS mutation (e.g., a mutation is at amino acid position 12, 13, or 61). In various embodiments of any of the above aspects or any other aspect of the invention delineated herein, the mutation is one or more of G12C, G12D, G12V, G12S, G13C, G13D, Q61H, and Q61L. In various embodiments of any of the above aspects or any other aspect of the invention delineated herein, the cancer contains a KRAS, HRAS, or NRAS mutation. In various embodiments of any of the above aspects or any other aspect of the invention delineated herein, the KRAS, HRAS, or NRAS mutation is Q61H.

In various embodiments of any of the above aspects or any other aspect of the invention delineated herein, the KRAS, HRAS, or NRAS mutation is a G12C or G12D mutation. In various embodiments of any of the above aspects or any other aspect of the invention delineated herein, the method further involves contacting the cell with 5-fluorouracil (5-FU) and leflunomide. In various embodiments of any of the above aspects or any other aspect of the invention delineated herein, the cancer cell contains one or more markers that is any one or more of carcinoembryonic antigen (CEA), carbohydrate antigen (CA) 19-9 (CA19-9), and carbohydrate antigen 15-3 (CA15-3). In various embodiments of any of the above aspects or any other aspect of the invention delineated herein, the cancer cell contains an increased level of creatine or phospho-creatine relative to the level of creatine or phospho-creatine present in a control cell. In various embodiments of any of the above aspects or any other aspect of the invention delineated herein, the method further involves contacting the cell with one or more additional chemotherapeutic agents that is any one or more of atovaquone, brequinar sodium, leflunomide, teriflunomide, BAY-2402234, AG-636, leucovorin, 5-fluorouracil, irinotecan, and oxaliplatin. In various embodiments of any of the above aspects or any other aspect of the invention delineated herein, the cell proliferation or survival is reduced by at least about 10% relative to an untreated cancer cell. In various embodiments of any of the above aspects or any other aspect of the invention delineated herein, the method further involves contacting the cell with 5-fluorouracil (5-FU) and leflunomide. In various embodiments of any of the above aspects or any other aspect of the invention delineated herein, the cancer cell contains a marker that is any one or more of carcinoembryonic antigen (CEA), carbohydrate antigen (CA) 19-9 (CA19-9), and carbohydrate antigen 15-3 (CA15-3). In various embodiments of any of the above aspects or any other aspect of the invention delineated herein, the cancer is characterized as having an increased level of creatine or phospho-creatine relative to the level of creatine or phospho-creatine present in a control cancer. In various embodiments of any of the above aspects or any other aspect of the invention delineated herein, the subject is administered R-GPA or a pharmaceutically acceptable salt thereof and a KRAS inhibitor that is any one or more of Sotorasib, Adagrasib, MRTX1133, RMC-6291, and RMC-6236. In various embodiments of any of the above aspects or any other aspect of the invention delineated herein, the method further involves administering to the subject an EGFR inhibitor. In various embodiments of any of the above aspects or any other aspect of the invention delineated herein, the subject is administered Adagrasib and R-GPA. In various embodiments of any of the above aspects or any other aspect of the invention delineated herein, treatment is monitored by detecting creatine excretion in urine, where an increase in such excretion indicates of SLC6A8 inhibition. In various embodiments of any of the above aspects or any other aspect of the invention delineated herein, R-GPA or its salt form is administered two times daily at a dosage of about 2400 mg or about 3000 mg (BID). In various embodiments of any of the above aspects or any other aspect of the invention delineated herein, the cancer is gastrointestinal (GI) adenocarcinoma or colorectal. In various embodiments of any of the above aspects or any other aspect of the invention delineated herein, the cancer contains a KRAS, HRAS, or NRAS mutation associated with constitutive GTPase activity. In various embodiments of any of the above aspects or any other aspect of the invention delineated herein, where prior to treatment the cancer developed resistance to one or more previous therapies. In various embodiments of any of the above aspects or any other aspect of the invention delineated herein, the cancer was resistant to treatment with oxaliplatin and/or capecitabine. In various embodiments of any of the above aspects or any other aspect of the invention delineated herein, the subject receives irinotecan, folinic acid, and 5-FU on Days 1 and 15 following commencement of treatment. In various embodiments of any of the above aspects or any other aspect of the invention delineated herein, irinotecan is administered intravenously at about 180 mg/m². In various embodiments of any of the above aspects or any other aspect of the invention delineated herein, folinic acid is administered intravenously at about 400 mg/m². In various embodiments of any of the above aspects or any other aspect of the invention delineated herein, 5-FU is administered intravenously at about 2400 mg/m². In various embodiments of any of the above aspects or any other aspect of the invention delineated herein, the intravenous infusion occurs over 46 hours. In various embodiments of any of the above aspects or any other aspect of the invention delineated herein, the method further involves administering bevacizumab. In various embodiments of any of the above aspects or any other aspect of the invention delineated herein, bevacizumab is administered at the commencement of treatment and 15 days following commencement of treatment. In various embodiments of any of the above aspects or any other aspect of the invention delineated herein, bevacizumab is administered at about 5 mg/kg. In various embodiments of any of the above aspects or any other aspect of the invention delineated herein, treatment is administered in a 28-day cycle. In various embodiments of any of the above aspects or any other aspect of the invention delineated herein, RGX-202-01 is administered twice a day at about 3000 mg on Days 1-28 of the 28-day cycle. In various embodiments of any of the above aspects or any other aspect of the invention delineated herein, bevacizumab is administered at a dose of about 5 mg/kg on Days 1 and 15 of each 28-day cycle. In various embodiments of any of the above aspects or any other aspect of the invention delineated herein, β-GPA is administered two times daily at a total daily dose of about 6000 mg/day on Days 1-28 of the 28-day cycle. In various embodiments of any of the above aspects or any other aspect of the invention delineated herein, irinotecan is administered at about 180 mg/m2 intravenously over 90 minutes concurrently with folinic acid (leucovorin), which is administered at 400 mg/m2 intravenously over 2 hours, followed by 5-FU, which is administered at about 2400 mg/m2 intravenously over 46 hours on Days 1 and 15 of each 28-day cycle. In various embodiments of any of the above aspects or any other aspect of the invention delineated herein, Bevacizumab is administered at about 5 mg/kg on Days 1 and 15 of each 28-day cycle.

In various embodiments of any of the above aspects or any other aspect of the invention delineated herein, treatment results in a reduction in target lesions. In various embodiments of any of the above aspects or any other aspect of the invention delineated herein, the target lesions are abdominal wall metastases. In various embodiments of any of the above aspects or any other aspect of the invention delineated herein, the reduction in target lesions is about a 20% or greater reduction. In various embodiments of any of the above aspects or any other aspect of the invention delineated herein, the cancer contains an increased level of creatine or phospho-creatine relative to the level of creatine or phospho-creatine present in a control cancer.

Any compositions or methods provided herein can be combined with one or more of any of the other compositions and methods provided herein.

Compositions and articles defined by the invention were isolated or otherwise manufactured in connection with the examples provided below. Other features and advantages of the invention will be apparent from the detailed description, and from the claims.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (3^{rd} edition. 2006); The Cambridge Dictionary of Science and Technology (Walker ed., 1990); The Glossary of Genetics, 5th Ed., R. Rieger et al. (eds.), Springer Verlag (1991); and Hale & Marham, The Harper Collins Dictionary of Biology (1991), The Biology of Cancer (2^{nd} edition, Weinberg et al., 2013), and Cancer: Principles and Practice of Oncology Primer of Molecular Biology in Cancer (3^{rd} edition, LWW, 2020). As used herein, the following terms have the meanings ascribed to them below, unless specified otherwise.

By “agent” is meant any small molecule chemical compound, antibody, nucleic acid molecule, or polypeptide, or fragments thereof.

By “alteration” or “modulation” is meant a change (increase or decrease) in the expression levels, structure, or activity of a gene or polypeptide as detected by standard art known methods such as those described herein. As used herein, an alteration includes a 10% change in expression levels, preferably a 25% change, more preferably a 40% change, and most preferably a 50% or greater change in expression levels.

By “ameliorate” is meant decrease, suppress, attenuate, diminish, arrest, or stabilize the development or progression of a disease.

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

By “chemotherapeutic agent” is meant any agent that inhibits cancer cell proliferation, inhibits cancer cell survival, inhibits or stabilizes tumor growth, or that is otherwise useful in the treatment of cancer. Exemplary anti-cancer agents provided herein can be used in combination with a creatine transport inhibitor (e.g., RGX-202, RGX-202-01) and/or a creatine kinase inhibitor provided herein to reduce cancer cell proliferation, or reduce tumor size in a subject.

Exemplary chemotherapeutic agents include, but are not limited to irinotecan, oxaliplatin, cetuximab, bevacizumab (AVASTIN®), leucovorin, and 5-fluorouracil (5-FU).

Chemotherapeutic agents as used herein encompass both chemical and biological agents. These agents function to inhibit a cellular activity upon which the cancer cell depends for continued survival. Categories of chemotherapeutic agents include alkylating/alkaloid agents, antimetabolites, hormones or hormone analogs, and miscellaneous antineoplastic drugs. Most if not all of these agents are directly toxic to cancer cells and do not require immune stimulation. In one embodiment, a chemotherapeutic agent is an agent of use in treating neoplasms such as solid tumors. In one embodiment, a chemotherapeutic agent is a radioactive molecule. One of skill in the art can readily identify a chemotherapeutic agent of use (e.g. see Slapak and Kufe, Principles of Cancer Therapy, Chapter 86 in Harrison's Principles of Internal Medicine, 14th edition; Perry et al., Chemotherapy, Ch. 17 in Abeloff, Clinical Oncology 2.sup.nd ed., COPYRGT. 2000 Churchill Livingstone, Inc; Baltzer L, Berkery R (eds): Oncology Pocket Guide to Chemotherapy, 2nd ed. St. Louis, Mosby-Year Book, 1995; Fischer D S, Knobf M F, Durivage H J (eds): The Cancer Chemotherapy Handbook, 4th ed. St. Louis, Mosby-Year Book, 1993). In embodiments, agents and combination described herein decrease cancer cell proliferation or survival by at least 10%, 20%, 30%, 40%, 50%, 60%, 70%, 80%, 90%, 95%, or 100%, and includes inducing cell death (apoptosis) in a cell or cells within a cell mass.

As used herein, the terms “O-Guanidinopropionic acid” or “β-GPA” refers to a small molecule having the structure:

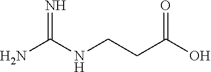

and salts thereof. β-GPA is a creatine analog identified by CAS number 353-09-3.β-Guanidinopropionic acid (0-GPA) can also be referred to as guanidinopropionic acid, beta-guanidinopropionic acid or, N-(aminoiminomethyl)-beta-alanine, which inhibits the creatine transporter by at least about 10%, 20%, 30%, 40%, 50%, 60%, 70%, 80%, 90%, or 100%. β-GPA, RGX-202, and RGX-202-01 are exemplary creatine transporter inhibitors.

“Biological sample” as used herein refers to a sample obtained from a biological subject, including a sample of biological tissue or fluid origin, obtained, reached, or collected in vivo or in situ, that contains or is suspected of containing nucleic acids or polypeptides associated with a cancer, e.g., creatine kinase B (CKB) or mutations in KRAS. A biological sample also includes samples from a region of a biological subject containing precancerous or cancer cells or tissues. Such samples can be, but are not limited to, organs, tissues, fractions and cells isolated from mammals including, humans such as a patient, mice, and rats. Biological samples also may include sections of the biological sample including tissues, for example, frozen sections taken for histologic purposes. A biological sample is typically of an eukaryotic origin, for example, a mammal (e.g., rat, mouse, cow, dog, guinea pig, rabbit, primate, for example, chimpanzees or humans).

By “colorectal cancer” or “CRC” is meant a cancer that affects the colon or rectum, located at the end of the gastrointestinal tract.

“Combination” therapy, as used herein, unless otherwise clear from the context, is meant to encompass administration of two or more therapeutic agents in a coordinated fashion, and includes, but is not limited to, serial or substantially concurrent dosing. Specifically, combination therapy encompasses both co-administration (e.g., administration of a co-formulation or simultaneous administration of separate therapeutic compositions) and serial or sequential administration, provided that administration of one therapeutic agent is conditioned in some way on administration of another therapeutic agent. For example, one therapeutic agent may be administered only after a different therapeutic agent has been administered and allowed to act for a prescribed period of time. See, e.g., Kohrt et al. (2011) Blood 117:2423.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

A “control sample” refers to a sample of biological material representative of healthy, cancer-free animals or animals with cancer. A sample either can be collected from an animal for the purpose of being used in the methods described in the present invention or it can be any biological material representative of normal, cancer-free animals suitable for use in the methods of this invention. A control sample also can be obtained from normal tissue from the animal that has cancer or is suspected of having cancer. A control sample also can refer to a given level of a marker representative of the cancer-free population, that has been previously established based on measurements from normal, cancer-free animals. Alternatively, a biological control sample can refer to a sample that is obtained from a different individual or be a normalized value based on baseline data obtained from a population. Further, a control sample can be defined by a specific age, sex, ethnicity or other demographic parameters. In some situations, the control is implicit in the particular measurement. A typical control level for a gene is two copies per cell. An example of an implicit control is where a detection method can only detect a marker, e.g., Creatine kinase B-type (CKB), or the corresponding gene copy number, when a level higher than that typical of a normal, cancer-free animal is present. Another example is in the context of an immunohistochemical assay where the control level for the assay is known. Other instances of such controls are within the knowledge of the skilled person.

As used herein, the term “creatine” refers to a molecule having the chemical structure:

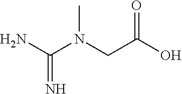

Creatine can rapidly resynthesize ATP from ADP through the anaerobic conversion of phosphorylated creatine (phosphocreatine) to creatine in a reversible reaction by the enzyme creatine kinase.

By “creatine transporter polypeptide” is meant a polypeptide or a fragment thereof having creatine transport activity. The creatine transporter polypeptide can be expressed on the outer membrane of a cell (e.g., a cancer cell). Exemplary creatine transporter polypeptides include solute carrier family 6 member 8 polypeptide (SLC6A8) and solute carrier family 16 member 12 polypeptide (SLC16A12).

By “creatine transport inhibitor” is meant an agent provided herein that the reduces or inhibits activity or reduces the level or expression of a creatine transporter polypeptide. Exemplary creatine transport inhibitors are listed in Table 2. In one embodiment, the creatine transport inhibitor is RGX-202, RGX-202-01, as well as analogs and derivatives thereof.

By “CKB” or “Creatine Kinase B polypeptide” is meant a cytoplasmic polypeptide involved in energy homeostasis or a fragment thereof having kinase activity, e.g., an enzyme capable of reversibly catalyzing the transfer of phosphate between ATP and various phosphogens such as creatine phosphate and having at least about 85% or greater amino acid sequence identity to NCBI Gene identification Gene ID: 1152; NCBI Reference Sequence: NP_001814.2. An exemplary human CKB amino acid sequence is provided below:

>NP_001814.2 creatine kinase B-type isoform 1 [Homo sapiens]
(SEQ ID NO: 1)
MPFSNSHNALKLRFPAEDEFPDLSAHNNHMAKVLTPELYAELRAKSTPSGFTLDDVIQTGVDNPGHPYIM
TVGCVAGDEESYEVFKDLFDPIIEDRHGGYKPSDEHKTDLNPDNLQGGDDLDPNYVLSSRVRTGRSIRGF
CLPPHCSRGERRAIEKLAVEALSSLDGDLAGRYYALKSMTEAEQQQLIDDHFLFDKPVSPLLLASGMARD
WPDARGIWHNDNKTFLVWVNEEDHLRVISMQKGGNMKEVFTRFCTGLTQIETLFKSKDYEFMWNPHLGYI
LTCPSNLGTGLRAGVHIKLPNLGKHEKFSEVLKRLRLQKRGTGGVDTAAVGGVFDVSNADRLGFSEVELV
QMVVDGVKLLIEMEQRLEQGQAIDDLMPAQK

The terms “decrease,” “reduced,” “reduction,” “decrease,” or “inhibit” are all used herein generally to mean a decrease by a statistically significant amount. However, for avoidance of doubt, “reduced”, “reduction” or “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g. absent level as compared to a reference sample), or any decrease between 10-100% as compared to a reference level.

“Detect” refers to identifying the presence, absence or amount of the analyte to be detected.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

The phrase “detecting a cancer” or “diagnosing a cancer” refers to determining the presence or absence of cancer or a precancerous condition in a subject. “Detecting a cancer” also can refer to obtaining indirect evidence regarding the likelihood of the presence of precancerous or cancerous cells in the animal or assessing the predisposition of a patient to the development of a cancer.

By “disease” is meant any condition or disorder that damages or interferes with the normal function of a cell, tissue, or organ. Examples of diseases include, but are not limited to, breast cancer, colorectal cancer (CRC), lung cancer (e.g., non-small cell lung cancer (NSCLC)), melanoma, pancreatic cancer, or gastrointestinal cancer. In some instances, the cancer is associated with a mutation in a KRAS, HRAS, or NRAS polypeptider.

By a “drug resistant” cancer is meant a cancer that does not respond, or exhibits a decreased response to, one or more chemotherapeutic agents.

By “effective amount” is meant the amount of an agent required to ameliorate the symptoms of a disease relative to an untreated subject (e.g., a subject that does not have cancer).

The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

The invention provides a number of targets that are useful for the development of highly specific drugs to treat or a disorder characterized by the methods delineated herein. In addition, the methods of the invention provide a facile means to identify therapies that are safe for use in subjects. In addition, the methods of the invention provide a route for analyzing virtually any number of compounds for effects on a disease described herein with high-volume throughput, high sensitivity, and low complexity.

The term “expression” refers to the biosynthesis of a gene product. For example, in the case of a structural gene, expression involves transcription of the structural gene into mRNA and the translation of mRNA into one or more polypeptides.

As used herein, the term “failed to respond to a prior therapy” or “refractory to a prior therapy,” refers to a cancer that progressed despite treatment with the therapy.

As used herein, the term “FOLFIRI” refers to a chemotherapy regimen, comprising leucovorin (folinic acid-FOL), 5-fluorouracil (F), and irinotecan hydrochloride (IRI). FOLFIRI is a standard treatment for gastrointestinal cancers (e.g., colorectal cancers).

As used herein, the term “FOLFOX” refers to a chemotherapy regimen, comprising leucovorin (folinic acid-FOL), 5-fluorouracil (F), and oxaliplatin (OX). FOLFOX is a standard treatment for gastrointestinal cancers (e.g., colorectal cancers).

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

By “HRAS” or “H-Ras” or “Harvey rat sarcoma polypeptide” or “HRAS polypeptide” is meant a polypeptide or a fragment thereof having at least about 85% or greater amino acid sequence identity to GenBank Accession No. CAG38816.1, which is reproduced below, and having GTPase activity. GTPase activity involves converting guanosine triphosphate (GTP) to guanosine diphosphate (GDP). Exemplary human HRAS amino acid sequences are provided below:

>CAG38816.1 HRAS [Homo sapiens]
(SEQ ID NO: 2)
MTEYKLVVVGAGGVGKSALTIQLIQNHFVDEYDPTIEDSYRKQVVIDGETCLLDILDTAGQEEY
SAMRDQYMRTGEGFLCVFAINNTKSFEDIHQYREQIKRVKDSDDVPMVLVGNKCDLAARTVESR
QAQDLARSYGIPYIETSAKTRQGVEDAFYTLVREIRQHKLRKLNPPDESGPGCMSCKCVLS

By “HRAS polynucleotide” is meant a polynucleotide encoding an HRAS polypeptide.

A “HRAS mutant cancer” as used herein refers to cancers comprising an alteration in the sequence of a HRAS polypeptide (i.e., an HRAS mutation).

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

The terms “increased”, “increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

As used herein, the term “in vitro” refers to events that occur in an artificial environment, e.g., in a test tube or reaction vessel, in cell culture, etc., rather than within a multi-cellular organism.

As used herein, the term “in vivo” refers to events that occur within a multi-cellular organism, such as a non-human animal.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “KRAS” or “K-Ras” or “Kirsten rat sarcoma polypeptide” or “KRAS polypeptide” is meant a polypeptide or a fragment thereof having at least about 85% or greater amino acid sequence identity to NCBI Gene identification 3845, NCBI Reference Sequence: NP_001356715.1 and having GTPase activity. GTPase activity involves converting guanosine triphosphate (GTP) to guanosine diphosphate (GDP). This definition is broad enough to encompass other RAS family members (e.g., HRAS and NRAS). Exemplary human KRAS amino acid sequences are provided below:

NCBI sequences:
>NP_001356715.1 GTPase KRas isoform a [Homo sapiens]
(SEQ ID NO: 3)
MTEYKLVVVGAGGVGKSALTIQLIQNHFVDEYDPTIEDSYRKQVVIDGETCLLDILDTAGQEEYSAMRDQ
YMRTGEGFLCVFAINNTKSFEDIHHYREQIKRVKDSEDVPMVLVGNKCDLPSRTVDTKQAQDLARSYGIP
FIETSAKTRQRVEDAFYTLVREIRQYRLKKISKEEKTPGCVKIKKCIIM
>NP_001356716.1 GTPase KRas isoform b [Homo sapiens]
(SEQ ID NO: 4)
MTEYKLVVVGAGGVGKSALTIQLIQNHFVDEYDPTIEDSYRKQVVIDGETCLLDILDTAGQEEYSAMRDQ
YMRTGEGFLCVFAINNTKSFEDIHHYREQIKRVKDSEDVPMVLVGNKCDLPSRTVDTKQAQDLARSYGIP
FIETSAKTRQGVDDAFYTLVREIRKHKEKMSKDGKKKKKKSKTKCVIM
UNIPROTKB-G3V5T7:
>tr|G3V5T7|G3V5T7_HUMAN GTPase KRas OS = Homo sapiens OX = 9606
GN = KRAS PE = 4 SV = 1
(SEQ ID NO: 5)
MTEYKLVVVGAGGVGKSALTIQLIQNHFVDEYDPTIEGVDDAFYTLVREIRKHKEKMSKD
GKKKKKKSKTKCVIM

In some embodiments, a KRAS polypeptide comprises an amino acid substitution of a cysteine for a glycine at amino acid position 12 (KRAS G12C), an amino acid substitution of an aspartic acid for a glycine at amino acid position 12 (KRAS G12D). The assignment of amino acid codon and residue positions for human KRas is based on the amino acid sequence identified by UniProtKB/Swiss-Prot P01116: Variant p.Glyl2Cys.

KRAS, HRAS, and NRAS are canonical ras gene family members that code for four highly related protein isoforms. These Ras family members share greater than 85% amino acid sequence identity and all share GTPase activity. Oncogenic mutations at positions 12, 13, or 61 of the H-ras, N-ras, and K-ras genes are among the most common genetic lesions in mammalian tumors. See, for example, Castellano and Santos, Genes Cancer. 2011 March; 2(3): 216-231, which is incorporated herein by reference.

By “KRAS polynucleotide” is meant a polynucleotide encoding a KRAS polypeptide.

A “KRAS mutant cancer” as used herein refers to cancers comprising an alteration in the sequence of a KRAS polypeptide (i.e., a KRAS mutation) or in the sequence of another RAS family member (e.g., HRAS, NRAS) falling under the definition of a KRAS polypeptide.

By “KRAS inhibitor” is meant an agent that reduces KRAS expression or activity. Such reduction may be by about 10%, 20%, 30%, 40%, 50%, 60%, 70%, 80%, 90%, or 100%. Exemplary KRAS inhibitors include but are not limited to those in Table 1 provided herein.

By “marker” is meant any protein or polynucleotide having an alteration in expression level or activity that is associated with a disease or disorder. In some embodiments, a marker is a cancer or tumor marker. Exemplary cancer markers include tumor antigens, such as carcinoembryonic antigen (CEA), carbohydrate antigen (CA) 19-9 or CAT9-9, and carbohydrate antigen 15-3 (CA15-3).

By “metabolic rewiring” is meant the process by which a cancer (e.g., tumor) alters the flux of metabolites in one or more metabolic pathways to increase tumor growth. The metabolic pathway can include, for example, biosynthesis of anabolic building blocks required for growth, such as nucleotides, amino acids and lipids.

The terms “malignancy,” “malignant condition,” “cancer,” or “tumor,” as used herein, refer to an uncontrolled growth of cells which interferes with the normal functioning of the bodily organs and systems. A subject that has a malignancy (i.e., cancer or a tumor) is a subject having objectively measurable malignant or cancer cells present in the subject's body. Included in this definition are benign and malignant cancers, as well as dormant tumors or micrometastatses. Cancers which migrate from their original location and seed vital organs can eventually lead to the death of the subject through the functional deterioration of the affected organs.

As used herein, “metastatic tumor” refers to a tumor or cancer in which the cancer cells forming the tumor have a high potential to or have begun to, metastasize, or spread from one location to another location or locations within a subject. In some embodiments, the metastasis occurs via the lymphatic system or via hematogenous spread, for example, creating secondary tumors within the subject. Such metastatic behavior may be indicative of malignant tumors. In some cases, metastatic behavior may be associated with an increase in cell migration and/or invasion behavior of the tumor cells.

Examples of cancers that can be defined as metastatic include but are not limited to: non-small cell lung cancer (NSCLC) (e.g., non-squamous non-small cell lung cancer), breast cancer, ovarian cancer, colorectal cancer, cholangial cancer, biliary tract cancer, bladder cancer, brain cancer including glioblastomas and medulloblastomas, cervical cancer, choriocarcinoma, endometrial cancer, esophageal cancer, gastric cancer, gastrointestinal cancer, hematological neoplasms, multiple myeloma, leukemia, intraepithelial neoplasms, liver cancer, lymphomas, neuroblastomas, oral cancer, pancreatic cancer, prostate cancer, sarcoma, skin cancer including melanoma, basocellular cancer, squamous cell cancer, testicular cancer, stromal tumors, germ cell tumors, thyroid cancer, and renal cancer.

As used herein, the term “modulate” is meant to refer to any change in biological state, i.e. increasing, decreasing, and the like.

By “NRAS” or “N-Ras” or “Kirsten rat sarcoma” or “NRAS polypeptide” is meant a polypeptide or a fragment thereof having at least about 85% or greater amino acid sequence identity to NCBI Gene identification 3845, NCBI Ref. Seq. Accession No. NP_002515.1, which is reproduced below, and having GTPase activity. GTPase activity involves converting guanosine triphosphate (GTP) to guanosine diphosphate (GDP).

>NP_002515.1 GTPase NRas [Homo sapiens]
(SEQ ID NO: 6)
MTEYKLVVVGAGGVGKSALTIQLIQNHFVDEYDPTIEDSYRKQVVIDGETCLLDILDTAGQEEY
SAMRDQYMRTGEGFLCVFAINNSKSFADINLYREQIKRVKDSDDVPMVLVGNKCDLPTRTVDTK
QAHELAKSYGIPFIETSAKTRQGVEDAFYTLVREIRQYRMKKLNSSDDGTQGCMGLPCVVM

By “NRAS polynucleotide” is meant a polynucleotide encoding a NRAS polypeptide.

A “NRAS mutant cancer” as used herein refers to cancers comprising an alteration in the sequence of a NRAS polypeptide (i.e., a NRAS mutation).

As used herein, “obtaining” as in “obtaining an agent” includes synthesizing, purchasing, or otherwise acquiring the agent.

As used herein, the term “pharmaceutical composition” refers to a mixture of at least one compound useful within the invention with other chemical components, such as carriers, stabilizers, diluents, dispersing agents, suspending agents, thickening agents, and/or excipients.

The pharmaceutical composition facilitates administration of the compound to an organism.

Multiple techniques of administering a compound exist in the art including, but not limited to, intravenous, oral, aerosol, parenteral, ophthalmic, pulmonary and topical administration.

As used herein, the term “pharmaceutically acceptable” refers to a material, such as a carrier or diluent, which does not abrogate the biological activity or properties of the composition, and is relatively non-toxic, i.e., the material may be administered to an individual without causing undesirable biological effects or interacting in a deleterious manner with any of the components of the composition in which it is contained.

The term “pharmaceutically acceptable carrier” includes a pharmaceutically acceptable salt, pharmaceutically acceptable material, composition or carrier, such as a liquid or solid filler, diluent, excipient, solvent or encapsulating material, involved in carrying or transporting a compound(s) of the present invention within or to the subject such that it may perform its intended function. Typically, such compounds are carried or transported from one organ, or portion of the body, to another organ, or portion of the body. Each salt or carrier must be “acceptable” in the sense of being compatible with the other ingredients of the formulation, and not injurious to the subject. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; diluent; granulating agent; lubricant; binder; disintegrating agent; wetting agent; emulsifier; coloring agent; release agent; coating agent; sweetening agent; flavoring agent; perfuming agent; preservative; antioxidant; plasticizer; gelling agent; thickener; hardener; setting agent; suspending agent; surfactant; humectant; carrier; stabilizer; and other non-toxic compatible substances employed in pharmaceutical formulations, or any combination thereof. As used herein, “pharmaceutically acceptable carrier” also includes any and all coatings, antibacterial and antifungal agents, and absorption delaying agents, and the like that are compatible with the activity of the compound, and are physiologically acceptable to the subject.

Supplementary active compounds may also be incorporated into the compositions.

As used herein, the language “pharmaceutically acceptable salt” refers to a salt of the administered compounds prepared from pharmaceutically acceptable non-toxic acids, including inorganic acids, organic acids, solvates, hydrates, or clathrates thereof.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

By “RAS polypeptide” is meant a RAS family member protein. Exemplary RAS proteins include, but are not limited, to HRAS, KRAS, or NRAS polypeptide. RAS polypeptides are described in E. Castellano and E. Santos, “Functional Specificity of Ras Isoforms,” Genes Cancer, 2:216-231 (2011), doi: 10.1177/1947601911408081, the disclosure of which is incorporated herein by reference in its entirety for all purposes.

By “RAS polynucleotide” is meant a polynucleotide encoding a RAS family member protein. Exemplary RAS polynucleotides include, but are not limited to, HRAS, KRAS, or NRAS polynucleotides.

By “RAS mutant cancer” is meant a cancer comprising an alteration in the sequence of a RAS polypeptide or polynucleotide.

By “reduces” is meant a negative alteration of at least 10%, 25%, 50%, 75%, or 100%.

By “reference” is meant a standard or control condition. For example, a cancer cell, population thereof, or a subject that has not been administered an agent provided herein.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

By “siRNA” is meant a double stranded RNA. Optimally, an siRNA is 18, 19, 20, 21, 22, 23 or 24 nucleotides in length and has a 2 base overhang at its 3′ end. These dsRNAs can be introduced to an individual cell or to a whole animal; for example, they may be introduced systemically via the bloodstream. Such siRNAs are used to downregulate mRNA levels or promoter activity.

By “SHP2” or “tyrosine phosphatase 2 polypeptide” or Src homology 2 (SH2)-containing protein tyrosine phosphatase 2 polypeptide” or “tyrosine-protein phosphatase non-receptor type 11 polypeptide” is meant a polypeptide or fragment thereof having phosphatase activity and having at least about 85% or greater amino acid sequence identity to NCBI Gene identification 5781, NCBI Reference Sequence: NP_002825.3. An exemplary human SHP2 amino acid sequence is provided below:

>NP_002825.3 tyrosine-protein phosphatase non-receptor type 11 isoform 1
[Homo sapiens]
(SEQ ID NO: 7)
MTSRRWFHPNITGVEAENLLLTRGVDGSFLARPSKSNPGDFTLSVRRNGAVTHIKIQNTGDYYDLYGGEK
FATLAELVQYYMEHHGQLKEKNGDVIELKYPLNCADPTSERWFHGHLSGKEAEKLLTEKGKHGSFLVRES
QSHPGDFVLSVRTGDDKGESNDGKSKVTHVMIRCQELKYDVGGGERFDSLTDLVEHYKKNPMVETLGTVL
QLKQPLNTTRINAAEIESRVRELSKLAETTDKVKQGFWEEFETLQQQECKLLYSRKEGQRQENKNKNRYK
NILPFDHTRVVLHDGDPNEPVSDYINANIIMPEFETKCNNSKPKKSYIATQGCLQNTVNDFWRMVFQENS
RVIVMTTKEVERGKSKCVKYWPDEYALKEYGVMRVRNVKESAAHDYTLRELKLSKVGQGNTERTVWQYHF
RTWPDHGVPSDPGGVLDFLEEVHHKQESIMDAGPVVVHCSAGIGRTGTFIVIDILIDIIREKGVDCDIDV
PKTIQMVRSQRSGMVQTEAQYRFIYMAVQHYIETLQRRIEEEQKSKRKGHEYTNIKYSLADQTSGDQSPL
PPCTPTPPCAEMREDSARVYENVGLMQQQKSFR

By “SHP2 inhibitor” is meant an agent provided herein that the reduces or inhibits activity or reduces the level or expression of SHP2.

By “SLC6A8” or “SLC6a8” or “solute carrier family 6 member 8 polypeptide” is meant a polypeptide or a fragment thereof having creatine transport activity and having at least about 85% or greater amino acid sequence identity to NCBI Gene identification 6535, NCBI Reference Sequence: NP_005620.1. Exemplary human SLC6A8 amino acid sequences are provided below:

>NP_005620.1 sodium- and chloride-dependent creatine transporter 1 isoform 1
[Homo sapiens]
(SEQ ID NO: 8)
MAKKSAENGIYSVSGDEKKGPLIAPGPDGAPAKGDGPVGLGTPGGRLAVPPRETWTRQMDFIMSCVGFAV
GLGNVWRFPYLCYKNGGGVFLIPYVLIALVGGIPIFFLEISLGQFMKAGSINVWNICPLFKGLGYASMVI
VFYCNTYYIMVLAWGFYYLVKSFTTTLPWATCGHTWNTPDCVEIFRHEDCANASLANLTCDQLADRRSPV
IEFWENKVLRLSGGLEVPGALNWEVTLCLLACWVLVYFCVWKGVKSTGKIVYFTATFPYVVLVVLLVRGV
LLPGALDGIIYYLKPDWSKLGSPQVWIDAGTQIFFSYAIGLGALTALGSYNRFNNNCYKDAIILALINSG
TSFFAGFVVFSILGFMAAEQGVHISKVAESGPGLAFIAYPRAVTLMPVAPLWAALFFFMLLLLGLDSQFV
GVEGFITGLLDLLPASYYFRFQREISVALCCALCFVIDLSMVTDGGMYVFQLFDYYSASGTTLLWQAFWE
CVVVAWVYGADRFMDDIACMIGYRPCPWMKWCWSFFTPLVCMGIFIFNVVYYEPLVYNNTYVYPWWGEAM
GWAFALSSMLCVPLHLLGCLLRAKGTMAERWQHLTQPIWGLHHLEYRAQDADVRGLTTLTPVSESSKVVV
VESVM
>NP_001136277.1 sodium- and chloride-dependent creatine transporter 1 isoform
2 [Homo sapiens]
(SEQ ID NO: 9)
MAKKSAENGIYSVSGDEKKGPLIAPGPDGAPAKGDGPVGLGTPGGRLAVPPRETWTRQMDFIMSCVGFAV
GLGNVWRFPYLCYKNGGGVFLIPYVLIALVGGIPIFFLEISLGQFMKAGSINVWNICPLFKGLGYASMVI
VFYCNTYYIMVLAWGFYYLVKSFTTTLPWATCGHTWNTPDCVEIFRHEDCANASLANLTCDQLADRRSPV
IEFWENKVLRLSGGLEVPGALNWEVTLCLLACWVLVYFCVWKGVKSTGKIVYFTATFPYVVLVVLLVRGV
LLPGALDGIIYYLKPDWSKLGSPQVWIDAGTQIFFSYAIGLGALTALGSYNRFNNNCYNGTSFFAGFVVF
SILGFMAAEQGVHISKVAESGPGLAFIAYPRAVTLMPVAPLWAALFFFMLLLLGLDSQFVGVEGFITGLL
DLLPASYYFRFQREISVALCCALCFVIDLSMVTDGGMYVFQLFDYYSASGTTLLWQAFWECVVVAWVYGA
DRFMDDIACMIGYRPCPWMKWCWSFFTPLVCMGIFIFNVVYYEPLVYNNTYVYPWWGEAMGWAFALSSML
CVPLHLLGCLLRAKGTMAERWQHLTQPIWGLHHLEYRAQDADVRGLTTLTPVSESSKVVVVESVM
>NP_001136278.1 sodium- and chloride-dependent creatine transporter 1 isoform
3 [Homo sapiens]
(SEQ ID NO: 10)
MKAGSINVWNICPLFKGLGYASMVIVFYCNTYYIMVLAWGFYYLVKSFTTTLPWATCGHTWNTPDCVEIF
RHEDCANASLANLTCDQLADRRSPVIEFWENKVLRLSGGLEVPGALNWEVTLCLLACWVLVYFCVWKGVK
STGKIVYFTATFPYVVLVVLLVRGVLLPGALDGIIYYLKPDWSKLGSPQVWIDAGTQIFFSYAIGLGALT
ALGSYNRFNNNCYKDAIILALINSGTSFFAGFVVFSILGFMAAEQGVHISKVAESGPGLAFIAYPRAVTL
MPVAPLWAALFFFMLLLLGLDSQFVGVEGFITGLLDLLPASYYFRFQREISVALCCALCFVIDLSMVTDG
GMYVFQLFDYYSASGTTLLWQAFWECVVVAWVYGADRFMDDIACMIGYRPCPWMKWCWSFFTPLVCMGIF
IFNVVYYEPLVYNNTYVYPWWGEAMGWAFALSSMLCVPLHLLGCLLRAKGTMAERWQHLTQPIWGLHHLE
YRAQDADVRGLTTLTPVSESSKVVVVESVM

By “specifically binds” is meant an agent that recognizes and binds a polypeptide of the invention, but which does not substantially recognize and bind other molecules in a sample, for example, a biological sample, which naturally includes a polypeptide of the invention.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987)Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 4° C. in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰° indicating a closely related sequence.

By “subject” is meant a mammal, including, but not limited to, a human or non-human mammal, such as a bovine, canine, equine, feline, ovine, or rodent.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

The term “tumor” refers to a tissue mass formed by or comprising cells undergoing uncontrolled proliferation. A tumor can be benign or malignant. A benign tumor is characterized as not undergoing metastasis. A malignant cell is a cancer cell and can undergo metastasis.

Tumors that can be treated with a therapy described herein (e.g., creatine inhibitor, alone or in combination with a KRAS inhibitor, including, without limitation, adenoma, angio-sarcoma, astrocytoma, epithelial carcinoma, germinoma, glioblastoma, glioma, hamartoma, hemangioendothelioma, hemangiosarcoma, hematoma, hepato-blastoma, leukemia, lymphoma, medulloblastoma, melanoma, neuroblastoma, osteosarcoma, retinoblastoma, rhabdomyosarcoma, sarcoma, and teratoma. The tumor can be chosen from acral lentiginous melanoma, actinic keratoses, adenocarcinoma, adenoid cystic carcinoma, adenomas, adenosarcoma, adenosquamous carcinoma, astrocytic tumors, bartholin gland carcinoma, basal cell carcinoma, bronchial gland carcinomas, capillary, carcinoids, carcinoma, carcinosarcoma, cavernous, cholangio-carcinoma, chondosarcoma, choroid plexus papilloma/carcinoma, clear cell carcinoma, cystadenoma, endodermal sinus tumor, endometrial hyperplasia, endometrial stromal sarcoma, endometrioid adenocarcinoma, ependymal, epitheloid, Ewing's sarcoma, fibrolamellar, focal nodular hyperplasia, gastrinoma, germ cell tumors, glioblastoma, glucagonoma, hemangiblastomas, hemangioendothelioma, hemangiomas, hepatic adenoma, hepatic adenomatosis, hepatocellular carcinoma, insulinoma, intaepithelial neoplasia, interepithelial squamous cell neoplasia, invasive squamous cell carcinoma, large cell carcinoma, leiomyosarcoma, lentigo maligna melanomas, malignant melanoma, malignant mesothelial tumors, medulloblastoma, medulloepithelioma, melanoma, meningeal, mesothelial, metastatic carcinoma, mucoepidermoid carcinoma, neuroblastoma, neuroepithelial adenocarcinoma nodular melanoma, oat cell carcinoma, oligodendroglial, osteosarcoma, pancreatic, papillary serous adeno-carcinoma, pineal cell, pituitary tumors, plasmacytoma, pseudo-sarcoma, pulmonary blastoma, renal cell carcinoma, retinoblastoma, rhabdomyosarcoma, sarcoma, serous carcinoma, small cell carcinoma, soft tissue carcinomas, somatostatin-secreting tumor, squamous carcinoma, squamous cell carcinoma, submesothelial, superficial spreading melanoma, undifferentiated carcinoma, uveal melanoma, verrucous carcinoma, vipoma, well differentiated carcinoma, and Wilm's tumor.

The term “tumor progression” refers to all stages of a tumor, including tumorigenesis, tumor growth and proliferation, invasion, and metastasis.

The term “inhibiting tumor progression” means inhibiting the development, growth, proliferation, or spreading of a tumor, including without limitation the following effects: inhibition of growth of cells in a tumor, (2) inhibition, to some extent, of tumor growth, including slowing down or complete growth arrest; (3) reduction in the number of tumor cells; (4) reduction in tumor size; (5) inhibition (i.e., reduction, slowing down or complete stopping) of tumor cell infiltration into adjacent peripheral organs and/or tissues; (6) inhibition (i.e. reduction, slowing down or complete stopping) of metastasis; (7) increase in the length of survival of a patient or patient population following treatment for a tumor; and/or (8) decreased mortality of a patient or patient population at a given timepoint following treatment for a tumor.

A tumor “responds” to a particular agent provided herein if tumor progression is inhibited as defined above.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

### BRIEF DESCRIPTION OF THE DRAWINGS

FIGS. 1A-1D show that RGX-202 and RGX-202-01 reduce cellular and tumoral creatine and phospho-creatine levels. FIGS. 1A-1D are graphs. FIG. 1A shows RGX-202 or vehicle control was injected (i.p.) into C57BL/6 wild type or SLC6A8 knock out mice, prior to injection of d3-creatine (i.p.). Mice were euthanized, hearts removed, and metabolites extracted for d3-creatine quantification by LC-MS/MS; n=3-8 independent experiments; p=0.0016 (100 mg/kg), p<0.0001 (250 mg/kg), p=0.0014 (500 mg/kg); mean +/− SEM. FIG. 1B shows UN-KPC-961 pancreatic tumor-bearing B6129SF1/J mice were fed a control or RGX-202 supplemented diet (800 mg/kg) for 35 days as soon as tumors reached ˜80 mm³. Mice were injected with 1 mg/kg d3-creatine (i.p.), tumors were extracted 1.5 hours later and d3-creatine levels were quantified by LC-MS/MS; n=4 per group, p<0.0001. FIGS. 1C-1D show correlation analysis of plasma creatine and RGX-202-01 exposure (AUC₀₋ₜ) (G) and average urine creatine and RGX-202-01 concentrations (H) measured over 24h in mice receiving either a control or RGX-202-01-supplemented diet at 100, 400 or 1200 mg/kg for 10 days; n=6 mice per dose group. Grey lines denote 95% confidence interval.

FIGS. 2A-2M show that SLC6A8 inhibition exhibits anti-tumor activity against primary and metastatic CRCs of different backgrounds. FIGS. 2A-2M show graphs and micrographs. FIGS. 2A and 2B show subcutaneous tumor growth (2A) and Kaplan-Meier survival curves (2B) by 1×10⁶ Lvm3b cells in athymic nude mice. Daily oral gavage of RGX-202-01 (200 mg/kg) started at a tumor size of ˜50 mm³, n=8-9 per group. Pictures show a control and RGX-202-01-treated mouse. FIG. 2C shows subcutaneous tumor growth of athymic nude mice injected with 0.5×10⁶ HCT116 cells. Treatment with a control diet or RGX-202-supplemented diet (800 mg/kg) started when tumors became palpable; n=5 per group. FIGS. 2D and 2E show subcutaneous tumor growth (D) and Kaplan-Meier survival curves (E) by 2×10⁶ HT29 cells in athymic nude mice receiving a control diet or RGX-202-01-supplemented diet (800 mg/kg) when tumors reached ˜55 mm³; insets represent growth curves for individual tumors; n=10 per group. FIG. 2F shows subcutaneous tumor growth by 0.5×10⁶ CT26 cells in BALB/c mice receiving a control diet or RGX-202-01-supplemented diet (500 mg/kg) starting at a tumor size of ˜100 mm³; n=7-8 per group. FIG. 2G shows subcutaneous tumor growth by 0.5×10⁶ MC38 cells in C57BL/6 mice. Mice received a control diet or RGX-202-01-supplemented diet (500 mg/kg) starting at a tumor size of ˜150 mm³; n=7 per group. FIGS. 2H and 21 show MC38 tumors treated with RGX-202-01 by oral gavage (QD 250 mg/kg p=0.0011; 500 mg/kg p=0.0032) or through diet (diet, p=0.0092) for 9 days were extracted and immunostained for cleaved caspase-3 (CC3) (H). Quantification of the percentage of tumor area stained with cleaved caspase-3; n=4 per group +/− SEM (I). FIGS. 2J and 2M show Representative images and quantification of control and RGX-202-treated Lvm3b xenografts immunostained with cleaved caspase-3 (J, K) and Ki67 (L, M) and counterstained with DAPI; n =3 tumors per group. FIGS. 2L and 2M show Quantification of CC3 (K) and Ki67 (M) positive cells in control and treated Lvm3b xenograft tumors; n=3 tumors per group. Mice were imaged on day 14 after injection; n=4 per group. Shown are mean +/− SEM (A, C, D, F, G, I, K, M). P values are based on two-sided t tests (A, C, D, F to I), one-sided t tests (K and M), Log-rank Mantel-Cox test (B and E. Scale bar (H, J and L) 200 μm.

FIGS. 3A-3K show that RGX-202 inhibits growth of KRAS wild-type and mutant CRC PDX and synergizes with 5-FU and leflunomide. FIGS. 3A-3K show graphs. FIGS. 3A-3D show subcutaneous tumor growth by ˜25 mm³ PDX fragments implanted in athymic nude mice receiving a control or RGX-202-supplemented diet (800 mg/kg) starting at a tumor size of ˜100 mm³ (CLR4), 250 mm³ (CLR7), 200 mm³ (CLR24) and 150 mm³ (CLR30); n=10 per group. FIG. 3E shows a waterfall plot of responses to RGX-202-01 across 43 colorectal PDX models; each bar represents an individual PDX and shading represent KRAS mutations. Mice were treated with control or RGX-202-01-supplemented diet at ˜400 mg/kg for 21 days. FIGS. 3F-3G show subcutaneous tumor growth (F) and Kaplan-Meier survival curves (G) of 1.5 ×10⁵ CT26 cells in BALB/c mice treated with control, RGX-202-formulated diet (800 mg/kg, p=0.0006), 5-FU (50 mg/kg/week) or a combination of RGX-202 and 5-FU; n=7-8 per group. FIG. 3H shows subcutaneous tumor growth of 2.5×10⁶ UN-KPC-961 cells in B6129SF1/J mice receiving control, RGX-202-formulated diet (800 mg/kg, p=0.021), gemcitabine (i.p. 100 mg/kg, p<0.0001) or a combination of RGX-202 and gemcitabine (p<0.0001); n=10 per group, *p (combination)=0.04, one-tailed t test. FIG. 3I shows subcutaneous tumor growth of 1×10⁶ MC38 cells in C57BL/6 mice receiving a control diet, RGX-202-supplemented diet (200 mg/kg), leflunomide (2.5 mg/kg) or a combination of RGX-202 and leflunomide; n=10 per group. FIG. 3J shows subcutaneous tumor growth by CLR1 PDX fragments implanted into athymic nude mice. Treatment with a control diet, a diet supplemented with 800 mg/kg RGX-202, leflunomide (7.5 mg/kg) or a combination of RGX-202 and leflunomide started when tumors reached ˜100 mm³, n=6 per group. FIG. 3K shows subcutaneous tumor growth by CLR28 PDX fragments in athymic nude mice. Treatment with a control diet, RGX-202-supplemented diet (800 mg/kg), leflunomide (7.5 mg/kg), a combination of RGX-202 and leflunomide or uridine (1 g/kg) and leflunomide started at −100 mm³; n=6 per group. Shown are means +/− SEM; p values are based on two-sided t tests (A to D, F, H to K) or Log-rank Mantel-Cox test (G).

FIGS. 4A-4I show tumoral CKB expression and creatine levels as predictive and pharmacodynamic biomarkers of SLC6A8 inhibition. FIGS. 4A-4I show a schematic, graphs, and micrographs. FIG. 4A shows a schematic showing the experimental design. FIGS. 4B-4D show tumor growth by 5×10⁶ HCT-8 (p=0.0004) (B), 5×10⁶ SW480 (p=0.01) (C) and 2×10⁶ Hs746T (D) cells subcutaneously injected into athymic nude mice receiving a control diet or RGX-202-01-supplemented diet (˜800 mg/kg). Pictures show IHC of CKB expression (brown), counterstained with Hematoxylin (blue) in the control tumors; n=8-10 per group. Shown are means +/− SEM; p values based on two-sided t tests. FIGS. 4E-4F show linear regression analyses of tumor growth inhibition (TGI) as a function of CKB mRNA (E) and TGI as a function of CKB tumor proportion score (TPS) (F). Each dot represents the mean value of data points from one xenograft model; n=5-7 tumors/model. FIG. 4G show non-parametric analysis of tumor growth inhibition on Day 21 relative to control from the 43 PDX models, stratified into models with low (0-5%) or >5% CKB TPS. FIGS. 4H-4I show correlation analysis of serum (H) or urine (I) creatine concentration and RGX-202 blood exposure (AUC₀₋ₜ). Dashed lines denote 95% confidence interval. Patients were sampled on Cycle 1 Day 15, n=13 patients total (H-I). Scale bar (B, C and D) 100 μm.

FIGS. 5A-5F show that RGX-202 inhibits tumor cell proliferation and liver colonization of metastatic CRC and pancreatic cell lines. FIGS. 5A-5F show graphs and micrographs. FIGS. 5A-5F show HCT-15 (A-C) or NCI-H508 (D to F) tumors treated with control or RGX-202-01 (800 mg/kg) (A, D, n=7-11/group) were dissected and immunostained for cleaved-caspase 3 (CC3) or Ki67 and counterstained with DAPI (B and E). Graphs showing quantification of CC3 and Ki67 positive cells in control and RGX-202-treated HCT-15 (C) or NCI-H508 (F) xenografts. N=3 tumors per group +/− SEM. Scale bar (B and E) 200 μm.

FIGS. 6A-6B show anti-tumor efficacy of RGX-202 and RGX-202-01 in combination with 5-FU and irinotecan. FIGS. 6A-6B show graphs. FIG. 6A shows tumor growth of 1.5×10⁵ CT26 cells subcutaneously injected into BALB/c mice. As soon as tumors reached an average of 50 mm³, mice were randomly distributed and received either a control diet, RGX-202-01-supplemented diet (˜500 mg/kg, p=0.092) or a combination of 5-FU (i.p. 30 mg/kg/week) and irinotecan (i.p. 15 mg/kg/week) (p=0.064) or a combination of RGX-202-01, 5-FU and irinotecan (p=0.0006); n=7-8 per group; shown are mean +/− SEM; All p values are based on two-sided t tests; *p=0.02. FIG. 6B shows body weight curves of BALB/c mice harboring CT26 tumors. Mice were treated with RGX-202 administered by oral gavage (500 mg/kg) or formulated in chow (500 mg/kg) and body weights were measured twice/week; n=7-11/cohort.

FIG. 7 shows graphs depicting xenograft tumor studies. Tumor growth of 4×10⁶ NCI-N87 (p=0.027), 2×10⁶ HepG2, 5×10⁶ Colo205 (p=0.042) and 1×10⁶ Lvm3b (p<0.0001) cells subcutaneously injected into athymic nude mice. Treatment with a control or -RGX-202-01-supplemented diet (˜800 mg/kg/day) started as soon as tumors reached 10-60 mm³; n=8-10 per group. All p values are based on two-sided t tests; n.s. =not significant. Shown are mean +/− SEM.

FIGS. 8A-8K show linear regression analysis demonstrates anti-tumor efficacy of RGX-202-01 in CKB high expressing cell lines. FIGS. 8A-8K show graphs and micrographs. FIGS. 8A-8C show linear regression analyses of the percentage of tumor growth inhibition (TGI) as a function of SLC6A8 mRNA (A), TGI as a function of CKB H-score (B) and CKB mRNA levels as a function of CKB H-score (C) in the tumors of the xenograft models tested. mRNA levels were measured by qPCR. Each dot represents the mean value of data points from one xenograft model, n=4-6 tumors/xenograft model. FIG. 8D shows qPCR analysis of CKB, CKM, CKMT1 and CKMT2 in the xenograft models tested. Relative expression is shown in ΔCt values; n=7-11 models; 4-5 tumors/models. Shown is the average +/− SEM. FIGS. 8E-8G show linear regression analysis of TGI as a function of CKM mRNA (E), CKMT1 mRNA (F) and CKMT2 (G) tumoral mRNA levels. mRNA levels were measured by qPCR; Each dot represents the mean value of data points from one xenograft model, n=4-5 tumors/xenograft model. FIGS. 8H-8I show linear regression analyses of the TGI and the fold change of cleaved caspase-3 (H) or Ki67 (I) positive tumor area assessed by immunohistochemistry (IHC) in RGX-202-01-treated tumors relative to control tumors; n=3 tumors/model. Each dot represents the mean value of data points from one xenograft model. FIGS. 8J-8K show CKB expression was assessed by IHC in 23 metastatic CRC specimens. Quantification of Tumor Proportion Score (TPS) is shown in (J) and representative images are shown in (K).

FIG. 9 provides a chart showing PDX models utilized in the PDX trial. Summary of PDX tumor growth data, KRAS and BRAF status. TGI(21)=Tumor growth inhibition on Day 21=((C₂₁−C₀)/C₂₁)−((T₂₁−T₀)/T₀). Change in tumor size relative to control.

FIG. 10 provides a chart showing information on cell lines. Anti-tumor efficacy and mutational status of the cell lines used in the xenograft studies. TGI=Tumor growth inhibition.

FIG. 11 shows a timeline depicting individual patient duration of treatment and clinical outcomes. The y-axis shows KRAS status and the x-axis show duration of treatment (weeks).

FIG. 12 shows a timeline depicting duration of treatment and best response in all evaluable patients on RGX-202-01 monotherapy and combination dose escalation.

FIG. 13 shows a graph demonstrating the anti-tumor activity in RGX-202-01 treated PDX models at a dose of 300 mg/kg. Tumor response was evaluated vs. control at day 21. CKB Tumor Proportion Score (TPS) was determined by immunohistochemistry.

FIG. 14 shows graphs demonstrating the RGX-202-mediated changes in creatine metabolism in patients with progressive disease (PD) vs. partial responders/stable disease (PR/SD) patients.

FIG. 15 shows graphs demonstrating the subject-level changes in creatine metabolism.

FIG. 16 shows chemical structures of exemplary creatine transporter inhibitors.

FIGS. 17A and 17B provide plots showing tumor growth inhibition achieved through administration of RGX-202 in combination with a low-dose KRAS inhibitor (i.e., MRTX849 (Adagrasib)). FIG. 17A provides a plot of tumor volume over time. FIG. 17B provides a plot of final tumor volume following treatment. In FIG. 17A the lines represent, from top-to-bottom, “control,” “RGX-202,” “MRTX849,” and “RGX-202+MRTX849.” In FIGS. 17A and 17B, * =p<0.05.

FIG. 18 provides plots showing tumor growth inhibition by RGX-202 in NCL-H460 KRASQ61 xenografts in mice. Tumor growth was measured as change in tumor volume or change in mouse body weight. 4×10⁶ NCI-H460 cells were injected in 6-8 week old female Nude mice. Treatment with RGX-202-formulated chow started when tumors reached ˜200 mm³.

FIG. 19 provides plots showing tumor growth inhibition by RGX-202 in NCL-H358 KRASG12C xenografts in mice. Tumor growth was measured as change in tumor volume or change in mouse body weight. 5×10⁶ NCI-H358 cells were injected in 6-8 week old female Nude mice. Treatment with RGX-202-formulated chow started when tumors reached ˜80 mm³.

### DETAILED DESCRIPTION OF THE INVENTION

As described below, the present invention features compositions and methods that are useful for the treatment of cancer (e.g., gastrointestinal, colorectal cancers) featuring creatine transporter inhibitors (e.g., β-Guanidinopropionic acid) in combination with a KRAS inhibitor or another chemotherapeutic agent (e.g., leucovorin (folinic acid-FOL), 5-fluorouracil (F), and irinotecan hydrochloride (IRI), oxaliplatin, and/or bevacizumab).

The invention is based, at least in part, on the discovery that an exemplary creatine transporter inhibitor β-Guanidinoproprionic acid (β-GPA), which is a creatine mimetic that competitively inhibits the creatine transporter SLC6A8, is useful alone or in combination with a KRAS inhibitor (e.g., Sotorasib, Adagrasib, MRTX1133, RMC-6291, and RMC-6236), a vascular endothelial growth factor inhibitor (e.g., bevacizumab), and/or FOLFIRI, to reduce cancer cell proliferation and/or induce cancer cell death, particularly in cells comprising a KRAS mutation or having increased levels of creatine or phospho-creatine.

Colorectal cancer (CRC) is a leading cause of cancer mortality. Creatine metabolism was previously shown to critically regulate colon cancer progression. As reported herein, β-GPA, an oral small-molecule SLC6A8 transporter inhibitor, robustly inhibits creatine import in vitro and in vivo, reduces intracellular phosphocreatine and ATP levels and induces tumor cell apoptosis in CRC. β-GPA suppressed tumor growth across KRAS wild-type and KRAS mutant xenograft, syngeneic and patient-derived xenograft colorectal cancers. Anti-tumor efficacy correlated with tumoral expression of creatine kinase B. Combining RGX-202 with 5-fluorouracil or the DHODH inhibitor leflunomide caused regressions of multiple colorectal xenograft and PDX tumors of distinct mutational backgrounds. β-GPA also perturbed creatine metabolism in patients with metastatic CRC enrolled in a Phase-1 trial, mirroring pharmacodynamic effects on creatine metabolism observed in mice. This is the first demonstration of pre-clinical and human pharmacodynamic activity for creatine metabolism targeting in oncology, revealing an important therapeutic target for CRC.

Accordingly, the compositions provided herein feature an orally bioavailable small-molecule creatine mimetic (β-GPA, RGX-202, RGX-202-01) that significantly inhibited the SLC6A8 transporter and suppressed the growth of colorectal cancer tumors in syngeneic, xenograft and PDX models and caused tumor cell apoptosis in vivo. β-GPA also strongly suppressed liver metastasis formation. Importantly, this therapy was effective in both KRAS wild-type tumors and tumors bearing various KRAS mutations, including the KRAS G12D allele, which is not currently druggable by clinical stage KRAS inhibitors alone. The anti-tumor efficacy by β-GPA also correlated with increased CKB expression in tumors.

Remarkably, the working examples provided herein demonstrate that β-GPA exhibited greater activity in combination with multiple standard of care regimens, including 5-FU, gemcitabine, FOLFIRI, and bevacizumab relative to single-agent efficacy. Thus, the anti-cancer therapies and agents provided herein are useful in combination with RGX-202 to reduce cancer cell proliferation, reduce tumor size, and improve clinical outcomes in patients with RAS mutant tumors (e.g., KRAS, HRAS, NRAS).

### Colorectal Cancer

During progression of colorectal cancer to liver metastasis, cancer cells upregulate creatine kinase brain-type (CKB) and secrete CKB into the extracellular space (Loo et al., Cell 160, 393-406 (2015)). CKB phosphorylates the metabolite creatine using ATP, thereby generating phosphocreatine. The y-phosphate of phosphocreatine has ˜50% greater free energy than the γ-phosphate of ATP. Phosphocreatine thus serves as a rapidly mobilizable high-energy phosphate reserve that can yield ATP in a reaction that does not require oxygen. Because of its high energy content, phosphocreatine is stored at high levels in metabolically active tissues such as muscle, brain and kidney-allowing tight maintenance of organismal ATP levels, which are critical for a vast number of metabolic and homeostatic processes. Although extracellular ATP levels are normally low, the death of cancer cells and stromal cell substantially increases extracellular ATP concentrations in the tumor microenvironment, which have been observed to exceed 700 μM. Excess of ATP over circulating creatine levels, which range from 9-90 PM, thus favors phosphocreatine generation in the tumor microenvironment-a reaction mediated by tumor-secreted CKB. Phosphocreatine import by SLC6A8 enhanced tumoral ATP levels and accordingly promoted cell survival under hypoxia (Loo et al., supra). Importantly, extracellular phosphocreatine supplementation rescued the metastatic defect and in vitro survival phenotype of CKB-depleted cells (Loo et al., supra). Gastrointestinal tumors, such as CRC and pancreatic cancers are highly hypoxic, as are metastases formed by these cancers. This metabolic axis provides a mechanism to support tumor growth in hypoxic environments. Importantly, the expression levels of CKB and SLC6A8 are associated with increased colorectal cancer metastasis in patients (Loo et al., supra).

The SLC6A8 transporter was identified a therapeutic target in colorectal cancer. The orally bioavailable small-molecule creatine mimetic (RGX-202, also called β-GPA) significantly inhibited SLC6A8 and suppressed the growth of colorectal cancer tumors in syngeneic, xenograft and PDX models and caused tumor cell apoptosis in vivo. RGX-202 also strongly suppressed liver metastasis formation. Importantly, it was shown that this therapy was effective in both KRAS wildtype tumors and tumors bearing various KRAS mutations, including the KRAS G12D allele, which is not currently druggable by clinical stage KRAS inhibitors. Anti-tumor efficacy by RGX-202 correlated with increased CKB expression in tumors. RGX-202 exhibited greater activity in combination with multiple standard of care regimens, including 5-FU and gemcitabine, relative to single-agent efficacy. It was also shown that RGX-202 synergized with the dihydroorotate dehydrogenase (DHODH) enzyme inhibitor leflunomide, an oral compound previously shown to suppress colorectal cancer cell growth under hypoxia by repressing nucleotide biosynthesis. Metabolic profiling studies revealed that RGX-202 suppressed intracellular phosphocreatine, creatine and ATP levels. Finally, a drug exposure-dependent increase of creatine in the blood and urine of mice and in patients treated with oral RGX-202 therapy was observed, confirming creatine transporter inhibition in patients and supporting further clinical testing of RGX-202 in later stage clinical trials.

### RGX-202

Using cell-based, ex vivo tissue-based, and in vivo assays, RGX 202 has been shown to inhibit the creatine transporter, SLC6a8, with a reported Ki of 8.8-120 μM (Dai 1999, Wyss 2000, Pereal 2002). RGX-202-mediated inhibition of the creatine transporter, SLC6a8, caused reduction of intracellular creatine levels in colon cancer cells in vitro and in tumors in vivo. RGX-202 treatment reduced cell viability in vitro (15-60%) and induced cell death in colon cancer cells in vitro (˜ 7-fold) and in tumors in vivo (5-15-fold). In embodiments, the methods provided herein include administering RGX-202 to a subject to treat a RAS mutant cancer (e.g., a KRAS, HRAS, or NRAS mutant cancer).

In syngeneic (immune-competent) and human xenograft (immune-deficient) mouse tumor models of colon cancer, gastric cancer and pancreatic cancer, RGX-202 inhibited primary tumor growth by 30-65%. The tumors tested included both KRAS wild-type and KRAS mutant colon cancer cells, KRAS mutant/p53 mutant pancreatic cancer cells. Long term treatment of human xenografts and syngeneic tumors with RGX-202 revealed 38-90% tumor regressions and demonstrated a reduced risk of death (Hazard ratio 0.1-0.3). In vivo tumor growth studies of 11 gastrointestinal xenografts demonstrated a positive correlation of anti-tumor efficacy of RGX-202 with CKB protein levels in these tumors; tumors that have high levels of CKB responded significantly better to RGX-202 treatment than tumors that had low or non-detectable levels of CKB. This suggests that response might be linked to CKB expression levels, consistent with the upregulation of the CKB/SLC6a8 pathway that is observed in metastatic colon cancer needed for tumor progression (Loo 2015). In addition, additive effects have been observed when RGX-202 is administered with 5-FU in vitro and in vivo and in combination with fluorouracil and irinotecan in vivo.

Furthermore, RGX-202 demonstrated additive tumor suppressive efficacy when administered in combination with gemcitabine in vivo. In vitro, treatment of HCT-116 colon cancer cells with RGX 202 reduced cell viability by ˜15-35%, while treatment with 5-FU showed only a modest and not significant decrease in cell viability; however, combination treatment with RGX 202 and 5-FU further reduced cell viability by ˜60-90% relative to control treatment and caused a significant synergistic effect in reducing cell viability relative to 5-FU alone. In vivo, combination treatment of RGX-202 and 5-FU inhibited CT-26 tumor growth by 99% (with 50% of mice displaying complete tumor regressions) and was significantly more effective than treatment with 5-FU alone or with RGX-202 alone. Additive anti-tumor activity has been also observed in pancreatic tumors in combination with gemcitabine treatment (˜80% tumor growth inhibition).

Anti-tumor efficacy of RGX-202 has been noted with various dosing paradigms including oral administration (by oral gavage once daily (QD) or supplemented in the diet) or subcutaneous administration (using osmotic pumps).

In tumor growth studies in rodents with RGX-202, the efficacious doses ranged from 50-500 mg/kg/day administered orally. At an efficacious dose of 200 mg/kg, systemic exposure (based on AUC β-24h estimates) to RGX-202 was similar in the chow administered group (144,810 ng-h/mL) relative to the oral gavage group (149,415 ng-h/mL), based on pharmacokinetic (PK) sampling data.

In animal models, RGX-202 inhibits primary tumor growth of various gastrointestinal cancers, including colorectal, pancreatic, and gastric, harboring different genetic backgrounds. As a single agent, RGX-202 inhibited growth of the human KRAS mutant in LvM3b, HCT 116 KRAS wild-type Colo 205 colon cancerand KRAS mutant CT26 and KRAS wild type MC38 murine colon cancers, and PANC1 human pancreatic cancer cells. Additionally, RGX-202 in combination with gemcitabine inhibited tumor growth of KPC murine pancreatic cancer, and RGX-202 in combination with both 5-fluorouracil and with 5-fluorouracil and irinotecan inhibited tumor growth of CT26 murine colon cancer. Tumor growth inhibitory activity of RGX-202 is intrinsically dependent on its ability to interfere with creatine metabolism. Importantly, other malignancies, including prostate, lung, ovary, cervical, and head and neck, have been noted for SLC6a8 and CKB gene amplification, potentially rendering them susceptible to RGX-202 inhibition of this metabolic pathway. Target engagement and clinical validity will be demonstrated by the effects of SLC6a8 inhibition by RGX-202-01 administration on metabolite levels, such as creatine, and by the levels of CKB expression by immunohistochemistry in tumor tissue. The validity of serum creatinine and creatine levels as pharmacodynamic markers of creatine import inhibition can be assessed by evaluating dose-responsiveness of changes in each of the markers after RGX-202-01 administration and combination therapies, such as RGX-202+a KRAS inhibitor from Table 1.

### RGX-202 Pharmacokinetics

The permeability coefficient of RGX-202 determined in human colon adenocarcinoma (Caco-2) cell monolayers was low. RGX-202 is not a substrate of the efflux transporters P-gp or BCRP in MDCKII cells or vesicles expressing P-gp or BCRP; however, there was evidence of active uptake of RGX-202 in the A to B direction in MDCKII cells, suggesting that RGX-202 is a substrate of an uptake transporter that is endogenously expressed in the MDCKII cell line. Although the endogenous transporter was not elucidated in that study, RGX-202 is a substrate for the amino acid transporter hPat1 (SLC36al). This transporter is involved in the cellular import of RGX-202 (i.e. endogenous β-GPA) in humans and is expressed predominantly in intestinal epithelial cells, brain, colon, liver and lungs (Metzner 2009).

RGX-202 is moderately bound to plasma proteins in all species. Overall mean percentage of bound RGX-202 ranged from 31.5% to 39.6%. In a pharmacology study in mice, after 17 days of dosing at an efficacious dose (800 mg/kg administered in the diet), there was substantial distribution of RGX-202 to the brain.

Metabolism does not appear to be a major clearance mechanism for this compound. RGX-202 does not appear to interact with cytochrome P450 enzymes and is not metabolized by liver hepatocytes from human, monkey, dog, rat and mouse. In addition, RGX-202 is not a substrate for any of the human CYP enzymes assayed (rCYP1A2, rCYP2A6, rCYP2B6, rCYP2C8, rCYP2C9, rCYP2C19, rCYP2D6, rCYP2E1, rCYP2J2, rCYP3A4 and rCYP4F2). There was no induction evident for 1A2, 2B6 or 3A and the IC₅₀ of RGX-202 for inhibition of CYP isoforms 2E1, 2A6, 1A2, 2B6, 2C19, 2C8, 2 C9, 2D6, 3A4/5 (midazolam and testosterone) was >100 μM, suggesting that RGX-202 is not an inhibitor or a very weak inhibitor of any of the cytochrome P450 isoforms tested. RGX-202 is also not an inhibitor of the human ABC transporters P-gp or BCRP. RGX-202 was also not an inhibitor of the human SLC transporters OATP1B1, OATP1B3, OAT1, OAT3, OCT2, MATE1, and MATE2-K.

Since RGX-202 does not appear to be metabolized, therefore excretion of the parent unchanged is likely the main clearance mechanism. In the DRF studies in the rat and dog, significant amounts of RGX-202 were found in the urine, indicating that renal excretion is a likely clearance mechanism for this compound. The potential for biliary excretion of RGX-202 has not been evaluated.

### FOLFIRI

FOLFIRI is a chemotherapy regimen consisting of irinotecan, leucovorin, and 5-fluorouracil. Irinotecan is a topoisomerase inhibitor, which prevents DNA from uncoiling and duplicating. 5-FU is a pyrimidine analog and antimetabolite which incorporates into DNA and arrests synthesis. Fluorouracil injection, a nucleoside metabolic inhibitor for intravenous administration.

Leucovorin (folinic acid) is a vitamin B derivative which increases the cytotoxicity of 5-FU. Leucovorin is one of several active, chemically reduced derivatives of folic acid.

Recommended dose modifications for FOLFIRI-related toxicity are presented below

| | Starting Dose | Dose Level -1 | Dose Level -2 |
|---|---|---|---|
| Drug | mg/m² | mg/m² | mg/m² |
| Irinotecan | 180 | 150 | 120 |
| Leucovorin Infusion | 400 | No change | No change |
| 5-FU Infusion | 2400 | 2000 | 1600 |

Bevacizumab Bevacizumab is a vascular endothelial growth factor inhibitor. Bevacizumab is a recombinant humanized monoclonal IgG1 antibody that contains human framework regions and murine complementarity-determining regions. Bevacizumab has an approximate molecular weight of 149 kDa. Bevacizumab is produced in a mammalian cell (Chinese Hamster Ovary) expression system. Bevacizumab binds VEGF and prevents the interaction of VEGF to its receptors (Flt-1 and KDR) on the surface of endothelial cells. The interaction of VEGF with its receptors leads to endothelial cell proliferation and new blood vessel formation in in vitro models of angiogenesis. Administration of bevacizumab to xenotransplant models of colon cancer in nude (athymic) mice caused reduction of microvascular growth and inhibition of metastatic disease progression. Bevacizumab is for intravenous use. AVASTIN® contains bevacizumab at a concentration of 25 mg/mL in either a 100 mg/4 mL or 400 mg/16 mL single-dose vial. Bevacizumab is administered intravenously 5 mg/kg every 2 weeks on an every 14-day schedule prior to FOLFIRI.

### Cancer and Metabolic Rewiring

Given the surprising results obtained with therapeutic combinations featuring an SLC6A8 transporter inhibitor (e.g., β-Guanidinoproprionic acid, RGX-202) and a KRAS inhibitor (e.g., Sotorasib, Adagrasib, MRTX1133, RMC-6291, and RMC-6236) and/or another chemotherapeutic (e.g., FOLFIRI, FOLFOX, bevacizumab, 5-fluorouracil, leflunomide, irinotecan, and/or oxaliplatin) one of skill in the art would expect that other cancers having such characteristics (e.g., expressing the creatine transporter SLC6A8, expressing increased levels of creatine, and/or KRAS mutations) could be treated using a similar approach.

Examples of cancer types include but are not limited to, carcinoma, lymphoma, blastoma, sarcoma, and leukemia. More particular examples of such cancers include, but are not limited to, basal cell carcinoma, biliary tract cancer; bladder cancer; bone cancer; brain and CNS cancer; breast cancer; cancer of the peritoneum; cervical cancer; choriocarcinoma; colon and rectum cancer; connective tissue cancer; cholangial cancer, cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); lymphoma including Hodgkin's and non-Hodgkin's lymphoma; melanoma; myeloma; neuroblastoma; oral cavity cancer (e.g., lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; as well as other carcinomas and sarcomas; as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome.

Cancers evolve multiple mechanisms to sustain proliferative growth and survival. One such adaptive mechanism is altered metabolism, commonly referred to as “metabolic rewiring.” By altering the flux of metabolites in various metabolic pathways, cancer cells enhance biosynthesis of anabolic building blocks required for growth such as nucleotides, amino acids and lipids. Certain metabolites, however, can become limiting during cancer progression-requiring their extracellular import through metabolic transporters. Metabolic signaling pathways that are upregulated in cancer are further discussed, e.g., in N. S. Chandel, “Navigating metabolism.” Cold Spring Harbor Laboratory Press, Cold Spring Harbor, New York, 2015, and T. Suzuki, et al., “Mutant KRAS drives metabolic reprogramming and autophagic flux in premalignant pancreatic cells.” Cancer Gene Ther, (2021), the teachings of each which are incorporated herein by reference in their entireties.

Several factors can increase a person's risk of developing cancer, which include but are not limited to inherited genetic mutations, acquired gene mutations, family history of cancer, obesity, diets that are high in red meats and processed meats, smoking, moderate to heavy alcohol use, history of colorectal polyps (adenomas), history of inflammatory bowel disease, history of having Lynch syndrome (hereditary non-polyposis colorectal cancer, or HNPCC, familial adenomatous polyposis (FAP), and/or type-2 diabetes. Additional risk factors for colorectal cancers are discussed, e.g., in Johnson C M et al., “Meta-analyses of colorectal cancer risk factors.” Cancer Causes Control. 2013 June;24(6):1207-22, the teachings of which are incorporated by reference in its entirety.

A cancer diagnosis can be determined by the symptoms exhibited by a subject as well as a variety of different laboratory and medical tests. The cancer diagnosis is generally confirmed by biopsy and histological analysis of the tumor to evaluate cell proliferation and the expression of tumor markers. The skilled practitioner can determine which tests are appropriate based on the symptoms exhibited by the subject and the affected organ.

The prognosis of colorectal cancer is related to the degree of penetration of the tumor through the bowel wall and the presence or absence of nodal involvement. Consequently, early detection and treatment are especially important. Currently, diagnosis is aided by the use of screening assays for fecal occult blood, sigmoidoscopy, colonoscopy and double contrast barium enemas. Treatment regimens are determined by the type and stage of the cancer, and include surgery, radiation therapy and/or chemotherapy.

Early-stage cancers are primarily treated by surgical resection or removal. Recurrence of colon cancer following surgery (the most common form of therapy) is a major problem and is often the ultimate cause of death. Larger tumors and those that have spread to lymph nodes are treated with a variety of chemotherapeutic agents that are administered in the post-surgical ‘adjuvant’ setting to reduce the risk of metastatic relapse. However, most metastatic patients has a very poor prognosis. For example, patients previously diagnosed with colorectal cancer that become metastatic eventually succumb to their disease, with a meager 5-year survival rate of 14%.

In the most lethal cases, cancer cells can break away from a primary tumor, penetrate into lymphatic and blood vessels, circulate through the bloodstream, and grow in a distant focus (metastasize) in normal tissues elsewhere in the body. Metastasis can be local or distant. Metastasis is a sequential process, contingent on tumor cells breaking off from the primary tumor, traveling through the bloodstream, and stopping at a distant site. At the new site, the cells establish a blood supply and can grow to form a life-threatening mass. Both stimulatory and inhibitory molecular pathways within the tumor cell regulate this behavior, and interactions between the tumor cell and host cells in the distant site are also significant.

Metastases are most often detected through diagnostic methods known in the art, such as magnetic resonance imaging (MRI) scans, computed tomography (CT) scans, blood and platelet counts, liver function studies, chest X-rays and bone scans in addition to the monitoring of specific symptoms.

The compositions and methods provided herein overcome the limitations of previous cancer therapies by targeting creatine transport signaling and ameliorating metabolic rewiring induced by cancer cells. Furthermore, the compositions provided herein are useful in treating a subject with a KRAS mutant cancer.

### RAS Mutations in Cancer and RAS Inhibition

The 3 canonical members of the Ras gene family (H-ras, N-ras, and K-ras) were identified more than a quarter century ago because of their frequent oncogenic activation in human tumors. They are the founding members of the wider Ras superfamily including more than 150 small GTPases, divided into at least 5 distinct subfamilies (Ras, Rho/Rac, Rab, Arf, and Ran) on the basis of primary sequence relationships. In particular, the Ras subfamily encompasses the H-ras, N-ras, and K-ras genes together with the closely related R-Ras TC21, Ra1, and Rap loci.

All Ras superfamily proteins share very similar molecular structures and a common ability to bind and hydrolyze guanine nucleotides. The Ras proteins are continually cycling between active (GTP bound) and inactive (GDP bound) conformational states dependent on structural changes occurring mostly in the 2 motile switch I and switch II regions, which are also responsible for the functional interactions of these proteins with negative (GAP) and positive (GEF) cellular regulators. The binary behavior aspects of these proteins enable them to function as molecular switches in a broad range of signaling processes related to the transduction of extracellular signals to the interior of cells. Oncogenic mutations at positions 12, 13, or 61 of the H-ras, N-ras, and K-ras genes are among the most common genetic lesions in mammalian tumors. These mutations result in significant impairment of the overall GTPase activity of the carrier Ras proteins and lock them into a constitutively activated state in which they signal to downstream effectors, even in the absence of extracellular stimuli.

Expression of the H-ras, N-ras, and K-ras genes is nearly ubiquitous and broadly conserved across species, although there are specific differences of expression levels depending on the tissue and the developmental stage under study. In particular, these 3 loci are known to code for 4 different protein isoforms (H-Ras, N-Ras, K-Ras4A, and K-Ras4B), the latter 2 resulting from alternative splicing of exon 4 of the K-ras locus. These 4 Ras isoform proteins are highly homologous regarding their primary amino acid sequence (˜80%), and the differences among them concentrated in the so-called hypervariable region (HVR) of their C-terminal domains. These mammalian ras genes are expressed in all cell lineages and organs, although there are differences in expression through prenatal and postnatal development, and certain adult tissues preferentially express one or other member of the family.

The mammalian Ras subfamily proteins (H-Ras, N-Ras, K-Ras4A, and K-Ras4B) are highly conserved across different species and play functionally significant roles in numerous cellular processes, including proliferation, differentiation, and cell death. The high number of Ras activators and effectors identified in mammalian cells places the Ras proteins at the crossroads of a staggering number of cellular signaling networks. Such a central role of Ras gene products in normal cell signaling is also consistent with the high frequency of oncogenic activation of ras genes in human cancers. The importance of Ras signaling in tumor initiation and maintenance is emphasized not only by the prevalence of ras mutations but also by the deregulation of many of its activator or effector pathways, thus affecting Ras pathway activity. Indeed, the study of the contribution of Ras signaling to tumor development has greatly improved current understanding of the molecular basis for the pathogenesis of many human cancers.

The most frequent mechanism of oncogenic activation involves point mutations affecting the interaction of Ras with guanine nucleotides. Mutations detected in naturally occurring ras oncogenes affect codons 12, 13, 59, and 61. These mutations result in inhibition of GTP hydrolysis, either by diminishing GTPase activity or (for codon 59) by modulating the rate of guanine nucleotide exchange.

Oncogenic ras mutations are found in a great variety of human cancers, although their incidence varies considerably with tumor type. Qualitatively, H-ras mutations have been reported in melanoma, bladder, thyroid, and mammary carcinoma; K-ras mutations have been found in bladder, ovarian, thyroid, lung, colon and rectum, and pancreatic carcinoma; neuroblastoma; rhabdomyosarcoma; and acute nonlymphocytic leukemia. Finally, N-ras mutations were also described in melanoma, thyroid carcinoma, teratocarcinoma, fibrosarcoma, neuroblastoma, rhabdomyosarcoma, Burkitt lymphoma, acute promyelocytic leukemia, T cell leukemia, and chronic myelogenous leukemia. Quantitative analysis demonstrates the preferential association of some of the ras oncogenes with specific forms of human tumors. Thus, K-ras-activating missense mutations are frequently detected in non-small cell lung cancer (15%-20%), colon adenomas (40%), and pancreatic adenocarcinomas (95%), making it the single most common mutationally activated human oncoprotein. Likewise, N-ras mutations are frequently present in hematological malignancies (20%-30%) such as acute myeloblastic leukemia. In contrast, other tumor types do not show any significant preference for a specific ras oncogene isoform. For example, more than half of malignant thyroid tumors (poorly differentiated or undifferentiated) harbor a mutation in K-ras, H-ras, or N-ras. Furthermore, mutations in all 3 ras isoforms may occur within the same tumor in some thyroid adenomas and carcinomas, suggesting that each isoform may contribute to different aspects of tumoral growth. Simultaneous mutations in K-ras and N-ras have also been detected in multiple myeloma. Finally, although ras mutations are rare in breast cancer, point mutations in H-ras or K-ras have been detected in primary carcinomas and in some mammary tumor-derived cell lines.

Kirsten rat sarcoma, also known as KRAS, is a proto-oncogene that encodes a small 21-kD guanosine triphosphate (GTP)/guanosine diphosphate (GDP) binding the protein involved in the regulation of the cellular response to many extracellular stimuli. The KRAS gene is located at 12p12.1, spans approximately 38 kb. KRAS normally functions in signal transduction cascades initiated by the binding of epidermal growth factor receptor (EGFR), hepatocyte growth factor, and insulin-like growth factor to their receptors. When activated wild-type KRAS binds GTP, this results in a conformational change that allows the protein to bind and activate over 20 known downstream effectors, including Raf, Braf, mTOR, MEK1 and 2, ERK, AKT, and PIK3CA. These downstream effectors exert many different effects, including apoptosis suppression, promotion of cell growth, cell transformation, angiogenesis, migration, and differentiation. KRAS functions as a molecular binary switch that alternates between a GTP-bound “active” state and a GDP-bound “off” state, with each state having a specific molecular conformation.

While KRAS has intrinsic GTPase activity, the hydrolysis rate constant is too low to be physiologically relevant, but specific “GTPase activating proteins” can increase hydrolysis roughly 100,000-fold. In turn, guanine nucleotide exchange factor proteins promote GTP binding by lowering the affinity of KRAS for bound GDP and catalyzing its replacement with GTP. Activating KRAS mutations are point mutations mostly affecting KRAS amino acid residues 12, 13, and 61, all of which decrease the intrinsic KRAS and GTPase activating protein-promoted GTP hydrolysis, resulting in constitutive KRAS activation.

KRAS mutations in cancers have been associated with a poorer survival and increased tumor aggressiveness. The prognostic significance of KRAS mutations are further discussed, e.g., Dinu D, et al. Prognostic significance of KRAS gene mutations in colorectal cancer--preliminary study. J Med Life. 2014; 7(4):581-587, the teachings of which is incorporated herein by reference in its entirety.

Colorectal cancers frequently arise from preneoplastic lesions through the activation of oncogenes (KRAS and BRAF) as well as the inactivation of tumor suppressor genes (APC, p16, p53, and DCC) and mismatch repair genes, such as MLH1 and MSH2 and, to a lower extent, PMS2 and hMSH6. Approximately 30%-40% of colon cancers carry a KRAS mutation.

KRAS gene mutations are also common in pancreatic cancer, lung adenocarcinoma, colorectal cancer, gall bladder cancer, thyroid cancer, and bile duct cancer and are associated with drug-resistance to standard of care chemotherapeutic regimens. Non-limiting examples of KRAS mutations that have been identified in human subjects include G12C, G12D, G12V, G12S, G13C, G13D, Q61H, and Q61L. Additional examples of KRAS mutation status in cancer are discussed, e.g., Aza A. Lyanova et al., “The KRAS mutation status and resistance to cetuximab in patients with squamous cell carcinoma of oral cavity.” Journal of Clinical Oncology (2020) 38:15; Erminia Massarelli et al., “KRAS Mutation Is an Important Predictor of Resistance to Therapy with Epidermal Growth Factor Receptor Tyrosine Kinase Inhibitors in Non-Small-Cell Lung Cancer.” Clin Cancer Res May 15 2007 (13) (10) 2890-2896; Westcott PM and To M.D., “The genetics and biology of KRAS in lung cancer.” Chin J Cancer. 2013; 32(2):63-70; Buscail, L., et al., “Role of oncogenic KRAS in the diagnosis, prognosis and treatment of pancreatic cancer.” Nat Rev Gastroenterol Hepatol 17, 153-168 (2020); and Polom K, et al., “KRAS Mutation in Gastric Cancer and Prognostication Associated with Microsatellite Instability Status.” Pathol Oncol Res. 2019 January;25(1):333-340, the teachings of each of which are incorporated herein by reference in their entireties.

Methods of identifying a KRAS mutant cancer provided herein are known in the art. For example, histological analysis, sequencing, high-resolution melting analysis (HRM), single-strand conformation polymorphism (SSCP), denaturing gradient gel electrophoresis (DGGE), denaturing high-performance liquid chromatography (DHPLC), array/strip analysis, and allele-specific PCR can be used. Sanger sequencing and pyrosequencing of PCR-amplified DNA are commonly employed in KRAS mutation analysis. Sanger sequencing of PCR-amplified genomic DNA is considered the gold-standard for identifying KRAS mutant cancers. PCR primers are used to amplify the genomic DNA immediately flanking known KRAS point mutations, e.g., those describe above.

Due to the evasiveness of KRAS mutant cancers and low survival rates in subjects with KRAS mutant tumors, several chemotherapeutic agents have been developed to specifically target the most common KRAS mutant tumors. Small molecules that covalently bind and inhibit the KRAS GT2C oncogenic driver variant, which is present in approximately 4% of colorectal tumors, have elicited clinical responses in patients harboring this mutant allele. Furthermore, inhibitors targeting down-stream KRAS signaling in RAS-mutant tumors have also been developed. Non-limiting examples of KRAS inhibitors listed in Table 1 below.

TABLE 1
Exemplary KRAS Inhibitors

| Name | Description | Indications | Chemical Structure |
|---|---|---|---|
| Sotorasib (LUMAKRAS ™, AMG-510) | Targets KRAS G12C preventing KRAS signaling in tumor cells and reducing tumor cell proliferation | Lung cancer | 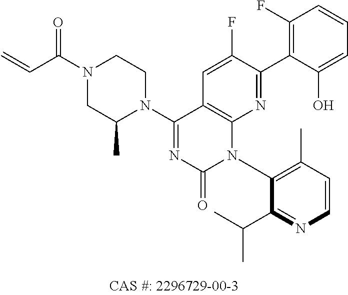 |
| Adagrasib (MRTX849) | Covalently binds mutant KRAS G12C | Lung, colorectal, pancreatic cancer, others | 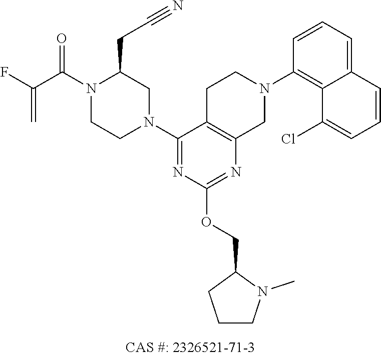 [SMILES: C=C(F)C(=O)N1CCN(c2nc(OC[C@@H]3CCCN3C)nc3c2CCN(c2cccc4cccc(Cl)c24)C3)C[C@@H]1CC#N] |
| MRTX1133 | MRTX1133 is a selective, reversible inhibitor and over 1000-fold more selective for the KRAS G12D than the normal (wild- type) KRAS | Lung, colorectal, pancreatic cancer, others | 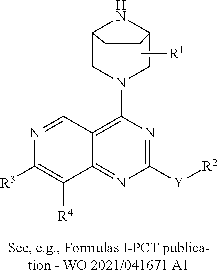 |
| RMC-6291 | Targets KRAS G12C and NRAS G12C | Lung, colorectal, pancreatic cancer, others | 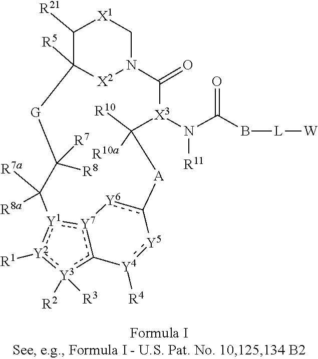 |
| RMC-6236 | Targets multiple | Lung, colorectal, | TBD |
| | RAS mutant | pancreatic cancer, | |
| | polypeptides | others | |

Additional KRAS inhibitors, analogs, and derivatives thereof are described, for example, in US Patent Nos. 8,546,4211BT; 10,640,504 B32; 10,125,134 B32; 10,125,134 B32; US Pg. No. 2020/0360374 A1; WO2021/041671 A1, the teachings of each of which are incorporated herein by reference in their entireties.

The KRAS inhibitors provided herein are administered to a subject in combination With an agent (e.g., RGX-202, RGX-202-01) the inhibits creatine transport or creatine signaling in cancer cells. The specific combination of KRAS inhibitors and creatine transport inhibitors can produce additive therapeutic effects in subjects With malignant cancers.

### Creatine Signaling in Cancer

Under normal physiological conditions, creatine is synthesized in the liver and kidney and transported throughout the body to tissues with high energy demands through an active transport system. Creatine is used by the body to rapidly resynthesize ATP from ADP through the anaerobic conversion of phosphorylated creatine (phosphocreatine) to creatine in a reversible reaction by the enzyme creatine kinase.

Creatine has the following chemical structure:

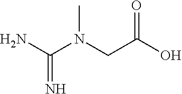

In times of low energy demand, excess ATP can be utilized to convert creatine to phosphocreatine. Because of its high energy content, phosphocreatine is stored at high levels in metabolically active tissues such as muscle, brain and kidney-allowing tight maintenance of organismal ATP levels, which are critical for a vast number of metabolic and homeostatic processes.

Cancers, such as colorectal and pancreatic cancers, are highly hypoxic as are the metastases formed by these cancers. During cancer proliferation and progression to liver metastasis, cancer cells upregulate creatine kinase brain-type (CKB) and secrete CKB into the extracellular space. CKB phosphorylates the metabolite creatine using ATP, thereby generating phosphocreatine. Increased expression of creatine kinase results in production of excess phosphocreatine which may be used as an energetic store for generating ATP needed to endure hepatic hypoxia. Specifically, the γ-phosphate of phosphocreatine has ˜50% greater free energy than the γ-phosphate of ATP. Phosphocreatine thus serves as a rapidly mobilizable high-energy phosphate reserve that can yield ATP in a reaction that does not require oxygen.

Although extracellular ATP levels are normally low, the death of cancer cells and stromal cell substantially increases extracellular ATP levels in the tumor microenvironment. Excess of ATP over circulating creatine levels, which range from about 9-90 μM, favors phosphocreatine generation in the tumor microenvironment-a reaction mediated by tumor-secreted CKB.

Phosphocreatine import by creatine transporters (e.g., SLC6A8) on the cell membrane of cancer cells further enhances tumoral ATP levels and promotes cancer cell survival under hypoxic conditions. Importantly, the working examples provided herein show that increased expression levels of CKB and SLC6A8 are associated with increased colorectal cancer metastasis and progressive disease in human patients. The working examples also demonstrate that targeting a creatine transporter with R-GPA and administering one or more additional chemotherapeutic agents (e.g., atovaquone, brequinar sodium, leflunomide, teriflunomide, BAY-2402234, AG-636, leucovorin, 5-fluorouracil, irinotecan, and oxaliplatin) can be highly beneficial in treating subjects with wild-type and mutant KRAS, HRAS, or NRAS tumors or previously drug-resistant tumors. Thus, the compositions provided herein can be used to ameliorate metabolic rewiring induced by cancer cells and reduce cell proliferation.

### SLC6A8

As discussed above, inhibition of the phosphocreatine system through inhibition of creatine uptake and/or creatine kinase in cancer cells is a useful target for the treatment of cancer and/or metastasis. One example of a creatine transporter that can be targeted to reduce cancer cell proliferation and/or increase cancer cell apoptosis is solute carrier family 6 member 8 polypeptide or SLC6A8.

SLC6A8 is a plasma membrane protein that functions to transport creatine into and out of cells. Defects in this gene can result in X-linked creatine deficiency syndrome. Under normal physiological conditions, SLC6A8 is highly expressed in the small intestine, heart, kidney, brain, and colon relative to other tissue types. Interestingly, SLC6A8 is one of the polypeptides upregulated in cancer cells as part of the metabolic rewiring process.

Specifically, in CKB-expressing cancer cells, phosphocreatine is imported into the cell via SLC6A8 which drives cancer cell survival in the hypoxic tumor niche. Knockdown of SLC6a8 in colon cancer cells has been shown to reduce intracellular phosphocreatine and ATP levels. Furthermore, when colon cancer cells are depleted of SLC6A8, metastatic activity is substantially reduced. Depleting SLC6a8 in CKB knockdown cells abrogated the protective effect of phosphocreatine during hypoxic stress. See e.g., in Loo J M, Scherl A, Nguyen A, et al. “Extracellular metabolic energetics can promote cancer progression.” Cell. 2015; 160(3):393-406, the teachings of which are incorporated herein by reference in its entirety.

### Therapeutic Compositions

Provided herein are compositions comprising therapeutic combinations featuring an SLC6A8 transporter inhibitor (e.g., β-Guanidinoproprionic acid, RGX-202) and a KRAS inhibitor (e.g., Sotorasib, Adagrasib, MRTX1133, RMC-6291, and RMC-6236) and/or another chemotherapeutic (e.g., FOLFIRI, FOLFOX, bevacizumab, 5-fluorouracil, leflunomide, irinotecan, and/or oxaliplatin). In some embodiments, a composition comprises RGX-202 in combination with a KRAS inhibitor. Such compositions reduce cancer cell proliferation and/or increase cancer cell apoptosis, and can be administered as a therapeutic treatment for cancer (e.g., colorectal cancer). Specifically, the agents provided herein inhibit creatine transport in a cell (e.g., a cancer cell, a neoplastic cell, a colorectal cell, liver cell, a kidney cell, etc.) and additional agents can be used to synergistically improve clinical outcomes, as shown in the working examples provided herein.

In one aspect, provided herein is a method of inhibiting cancer cell growth or proliferation, the method comprising:
a. administering to a cancer cell one or more creatine transporter inhibitor; and
b. administering to a cancer cell one or more antiproliferative agent.

In another aspect, provided herein is a pharmaceutical composition comprising: (a) an effective amount of a creatine transporter inhibitor, or a pharmaceutically acceptable salt thereof; and (b) an effective amount of one or more additional antiproliferative agent.

In some embodiments of any of the aspects, the creatine transporter inhibitor is a compound comprising the structure of

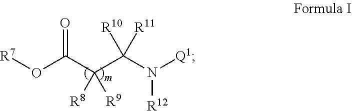

wherein Q¹ is optionally substituted amidino or optionally substituted 2-pyridyl;
m is 1 or 2;
R⁷ is hydrogen, optionally substituted C1-C6 alkyl, or optionally substituted C6-C10 aryl C1-C6 alkyl;
R⁸ and R⁹ are independently hydrogen, deuterium, halo, hydroxyl, NH2, optionally substituted C1-C3 alkyl, or R⁸ and R⁹ combine with the atoms to which they are attached to form an optionally substituted C3-C6 cycloalkyl ring; or R⁸ or R⁹ combine with R¹⁰ or R¹¹ with the atoms to which they are attached to form an optionally substituted C3-C4 cycloalkyl ring; or R⁸ or R⁹ combine with R¹² with the atoms to which they are attached to form an optionally substituted C3-C5 heterocycle;
R¹⁰ and R¹¹ are independently hydrogen, deuterium, optionally substituted C1-C4 alkyl, optionally substituted C2-C6 alkenyl, optionally substituted C2-C6 alkynyl or R¹⁰ and R¹¹ combine with the atoms to which they are attached to form an optionally substituted C3-C6 cycloalkyl ring; or R¹⁰ or R¹¹ combine with R⁸ or R⁹ with the atoms to which they are attached to form an optionally substituted C3-C4 cycloalkyl ring; or R¹⁰ or R¹¹ combine with R¹² with the atoms to which they are attached to form an optionally substituted C3-C4 heterocycle;
R¹² is hydrogen, optionally substituted C1-C6 alkyl, or R¹² combines with R⁸ or R⁹ with the atoms to which they are attached to form an optionally substituted C3-C5 heterocycle, or R¹² combines with R¹⁰ or R¹¹ with the atoms to which they are attached to form an optionally substituted C3-C4 heterocycle
wherein if m is 1 and R⁸ is hydrogen, halo, hydroxyl, or methyl then at least one of R⁹, R¹⁰, and R¹¹ is not hydrogen;
wherein if m is 1 and R¹⁰ is methyl then at least one of R⁸, R⁹, and R¹¹ is not hydrogen; wherein if m is 1 and R⁸ is NH-2 and R¹⁰ is hydrogen, methyl, or —CH2CH2OH then at least one of R9 or R¹¹ is not hydrogen;
wherein if m is 1, R⁸ is halo, and R10 is optionally substituted C1-C4 alkyl then at least one of R⁹ and R¹⁰ is not hydrogen;
or a pharmaceutically acceptable salt thereof.
In some embodiments ofany of the aspects, the creatine transporter inhibitor is selected from one of the agents in Table 2 provided below.

TABLE 2
Creatine Transporter and Creatine Kinase Inhibitors

| Name | Structure |
|---|---|
| Formula I | 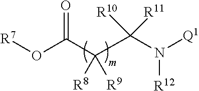 |
| | wherein Q¹ is optionally sub- |
| | stituted amidino or |
| | optionally substituted 2- |
| | pyridyl; m is 1 or 2; R⁷ is |
| | hydrogen, optionally sub- |
| | stituted C1-C6 alkyl, or |
| | optionally substituted C6- |
| | C10 aryl C1-C6 alkyl; R⁸ |
| | and R⁹ are independently |
| | hydrogen, deuterium, halo, |
| | hydroxyl, NH2, optionally |
| | substituted C1-C3 alkyl, or |
| | R⁸ and R⁹ combine with the |
| | atoms to which they are |
| | attached to form an option- |
| | ally substituted C3-C6 |
| | cycloalkyl ring; or R⁸ or R⁹ |
| | combine with R¹⁰ or R¹¹ with |
| | the atoms to which they are |
| | attached to form an option- |
| | ally substituted C3-C4 cyclo- |
| | alkyl ring; or R⁸ or R⁹ combine |
| | with R¹² with the atoms to which |
| | they are attached to form an |
| | optionally substituted C3-C5 |
| | heterocycle; R¹⁰ and R¹¹ are |
| | independently hydrogen, |
| | deuterium, optionally sub- |
| | stituted C1-C4 alkyl, option- |
| | ally substituted C2-C6 |
| | alkenyl, optionally substituted |
| | C2-C6 alkynyl or R¹⁰ and R¹¹ |
| | combine with the atoms to |
| | which they are attached to |
| | form an optionally sub- |
| | stituted C3-C6 cycloalkyl |
| | ring; or R¹⁰ or R¹¹ combine |
| | with R8 or R9 with the atoms |
| | to which they are attached to |
| | form an optionally sub- |
| | stituted C3-C4 cycloalkyl |
| | ring; or R¹⁰ or R¹¹ combine |
| | with R12 with the atoms to |
| | which they are attached to |
| | form an optionally substituted |
| | C3-C4 heterocycle; R¹² is |
| | hydrogen, optionally sub- |
| | stituted C1-C6 alkyl, or R¹² |
| | combines with R⁸ or R⁹ with |
| | the atoms to which they are |
| | attached to form an option- |
| | ally substituted C3-C5 hetero- |
| | cycle, or R¹² combines with |
| | R¹⁰ or R¹¹ with the atoms to |
| | which they are attached to form |
| | an optionally substituted C3- |
| | C4 heterocycle wherein if m |
| | is 1 and R⁸ is hydrogen, halo, |
| | hydroxyl, or methyl then at least |
| | one of R9, R¹⁰, and R¹¹ is not |
| | hydrogen; wherein if m is 1 and |
| | R¹⁰ is methyl then at least one of |
| | R8, R9, and R¹¹ is not hydro- |
| | gen; wherein if m is 1 and R⁸ is |
| | NH₂ and R¹⁰ is hydrogen, meth- |
| | yl, or —CH₂CH₂OH then at |
| | least one of R9 or R¹¹ is not |
| | hydrogen; wherein if m is 1, R8 |
| | is halo, and R10 is optionally |
| | substituted C1-C4 alkyl then at |
| | least one of R9 and R¹⁰ is not |
| | hydrogen; or a pharmaceutically |
| | acceptable salt thereof. |
| Formula II β-Guanidinopropionic acid (also referred to herein as β-GPA or ″RGX-202″), | 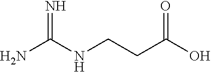 |
| Formula III | 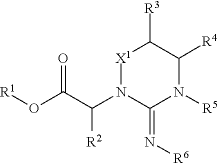 |
| | wherein X1 is absent, NH, or CH2; |
| | R¹ is hydrogen, optionally sub- |
| | stituted C1-C6 alkyl, or option- |
| | ally substituted C6-C10 aryl C1- |
| | C6 alkyl; R², R³, and R⁴ are |
| | independently hydrogen or option- |
| | ally substituted C1-C6 alkyl; |
| | and R⁵ and R⁶ are hydrogen or |
| | NH2; wherein if R⁵ and R⁶ are |
| | both hydrogen or R⁵ is NH₂ and |
| | R⁶ is hydrogen then R² is |
| | optionally substituted C1-C6 |
| | alkyl, or a pharmaceutically |
| | acceptable salt thereof. |
| Formula IV | 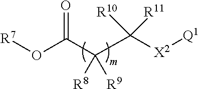 |
| | wherein Q¹ is optionally sub- |
| | stituted amidino or optionally |
| | substituted 2-pyridyl; X² is |
| | S or NR¹²; m is 0 or 1; R⁷ is |
| | hydrogen, optionally sub- |
| | stituted C₁-C₆ alkyl, or option- |
| | ally substituted C₆-C₁₀ aryl |
| | C₁-C₆ alkyl; R⁸ and R⁹ are |
| | independently hydrogen, |
| | deuterium, halo, hydroxyl, |
| | N H₂, optionally substituted |
| | C₁-C₆ alkyl, or R⁸ or R⁹ can |
| | combine with R¹⁰ or R¹¹ to |
| | form an optionally substituted |
| | C₃-C₆ cycloalkyl ring or with |
| | R¹² to form an optionally sub- |
| | stituted C₃-C₆ heterocycle; |
| | R¹⁰ and R¹¹ are independently |
| | hydrogen, deuterium, optionally |
| | substituted C₁-C₆ alkyl, or R¹⁰ |
| | or R¹¹ can combine with R⁸ or |
| | R⁹ to form an optionally sub- |
| | stituted C₃-C₆ cycloalkyl ring; |
| | R¹² is hydrogen, optionally sub- |
| | stituted C₁-C₆ alkyl, or R¹² can |
| | combine with R⁸ or R⁹ to form |
| | an optionally substituted C₃-C₆ |
| | heterocycle, wherein if Q¹ is |
| | optionally substituted 2-pyridyl |
| | then R¹² is hydrogen, and |
| | wherein if R⁹ is halo then R⁸ is |
| | halo or optionally substituted |
| | C₁-C₆ alkyl, or a pharmaceutic- |
| | ally acceptable salt thereof. |
| 2-(2-iminoimidazolidin- 1-yl)propanoic acid | 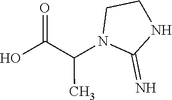 |
| 2-(2-iminoimidazolidin- 1-yl)butanoic acid | 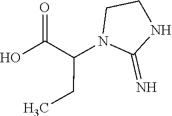 |
| 2-(2-iminoimidazolidin- 1-yl)-3-methylbutanoic acid | 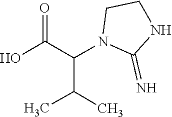 |
| 2-(2-iminoimidazolidin- 1-yl)-3-methylpentanoic acid | 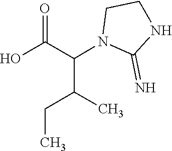 |
| 2-[3-amino-2-hydrazin- ylideneimidazolidin- 1-yl]acetic acid | 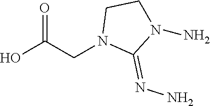 [SMILES: N/N=C1\N(N)CCN1CC(=O)O] |
| 2-[3-amino-2-hydrazin- ylideneimidazolidin- 1-yl]propanoic acid | 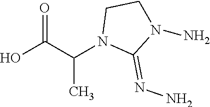 |
| 2-[3-amino-2-hydrazin- ylideneimidazolidin- 1-yl]-3-methylbutanoic acid | 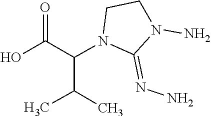 |
| 2-(3-amino-2-imino- imidazolidin-1-yl)- propanoic acid | 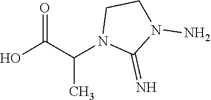 |
| 2-(3-amino-2-imino- imidazolidin-1-yl)- 3-methylbutanoic acid | 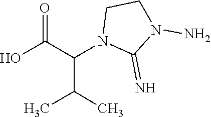 |
| 2-[2-hydrazinylidene- imidazolidin-1-yl]- acetic acid | 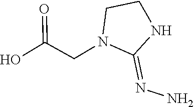 [SMILES: N/N=C1\NCCN1CC(=O)O] |
| 2-[2-hydrazinylidene- imidazolidin-1-yl]- propanoic acid | 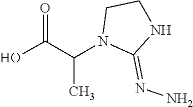 |
| 2-[2-hydrazinylidene- imidazolidin-1-yl]- 3-methylbutanoic acid | 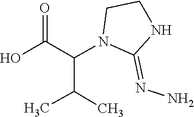 |
| 2-(2-imino-1,3-diazinan- 1-yl)propanoic acid | 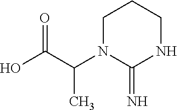 |
| 2-(2-imino-1,3-diazinan- 1-yl)butanoic acid | 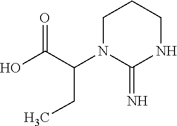 |
| 2-(2-imino-1,3-diazinan- 1-yl)-3-methylbutanoic acid | 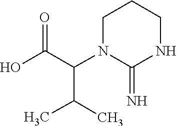 |
| 2-(2-imino-1,3-diazinan- 1-yl)-3-methylpentanoic acid | 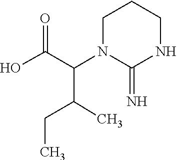 |
| 2-[3-amino-2-hydrazin- ylidene-1,3-diazinan- 1-yl]acetic acid | 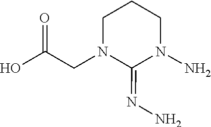 [SMILES: N/N=C1\N(N)CCCN1CC(=O)O] |
| 2-[3-amino-2-hydrazin- ylidene-1,3-diazinan- 1-yl]-3-methylbutanoic acid | 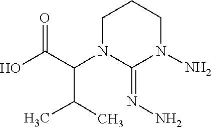 [SMILES: CC(C)C(C(=O)O)N1CCCN(N)/C1=N\N] |
| 2-(3-amino-2-imino- 1,3-diazinan-1-yl)- propanoic acid | 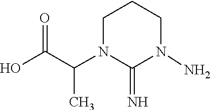 |
| 2-(3-imino-1,2,4- triazinan-2-yl)acetic acid | 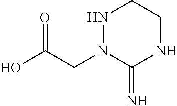 [SMILES: N=C1NCCNN1CC(=O)O] |
| 2-(3-imino-1,2,4-triazi- nan-2-yl)propanoic acid | 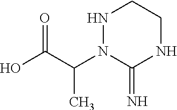 |
| 2-(3-imino-1,2,4-triazi- nan-2-yl)-3-methyl- butanoic acid | 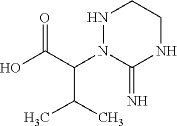 |
| cis-2-carbamimid- amidocyclopropane- 1-carboxylic acid | 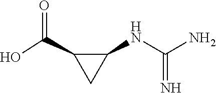 |
| trans-2-carbamimid- amidocyclopropane- 1-carboxylic acid | 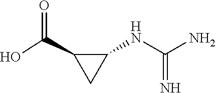 |
| 3-carbamimidamido- (2H)propanoic acid | 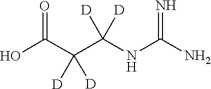 [SMILES: [2H]C([2H])(NC(=N)N)C([2H])([2H])C(=O)O] |
| 3-carbamimidamido- (3,3-2H2)propanoic acid | 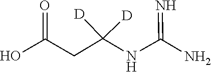 [SMILES: [2H]C([2H])(CC(=O)O)NC(=N)N] |
| 3-carbamimidamido- 2,2-difluoropropanoic acid | 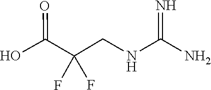 [SMILES: N=C(N)NCC(F)(F)C(=O)O] |
| 1-carbamimidoyl- azetidine-3-carboxylic acid | 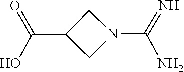 |
| cis-2-carbamimid- amidocyclobutane-1- carboxylic acid | 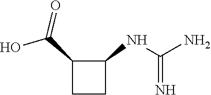 [SMILES: N=C(N)N[C@H]1CC[C@H]1C(=O)O] |
| trans-2-carbamimid- amidocyclobutane- 1-carboxylic acid | 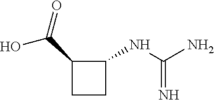 [SMILES: N=C(N)N[C@@H]1CC[C@H]1C(=O)O] |
| 3-guanidino-4,4,4- trifluorobutanoic acid | 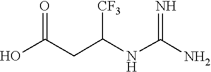 [SMILES: N=C(N)NC(CC(=O)O)C(F)(F)F] |
| 2-amino-3-guanidino- 4,4,4-trifluorobutanoic acid | 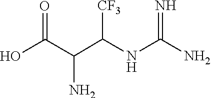 [SMILES: N=C(N)NC(C(N)C(=O)O)C(F)(F)F] |
| (1R,2S)-2-carbamimid- amidocyclopropane-1- carboxylic acid | 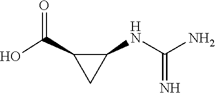 |
| (1S,2R)-2-carbamimid- amidocyclopropane-1- carboxylic acid | 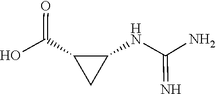 |
| (1R,2R)-2-carbamimid- amidocyclopropane-1- carboxylic acid | 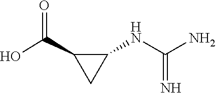 |
| (15,2S)-2-carbamimid- amidocyclopropane-1- carboxylic acid | 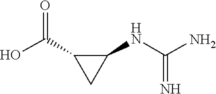 |
| (1R,2S)-2-carbamimid- amidocyclobutane-1- carboxylic acid | 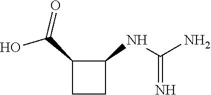 [SMILES: N=C(N)N[C@H]1CC[C@H]1C(=O)O] |
| (1S,2R)-2-carbamimid- amidocyclobutane-1- carboxylic acid | 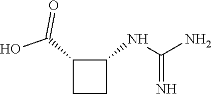 [SMILES: N=C(N)N[C@@H]1CC[C@@H]1C(=O)O] |
| (1R,2R)-2-carbamimid- amidocyclobutane-1- carboxylic acid | 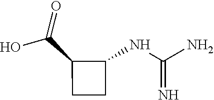 [SMILES: N=C(N)N[C@@H]1CC[C@H]1C(=O)O] |
| (1S,2S)-2-carbamimid- amidocyclobutane-1- carboxylic acid | 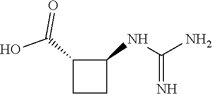 [SMILES: N=C(N)N[C@H]1CC[C@@H]1C(=O)O] |
| (2R,3R)-1-carbamimido- yl-2-methylazetidine-3- carboxylic acid | 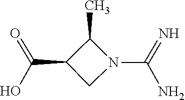 |
| (2R,3S)-1-carbamimido- yl-2-methylazetidine-3- carboxylic acid | 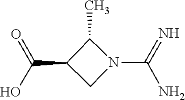 |
| (2S,3R)-1-carbamimido- yl-2-methylazetidine-3- carboxylic acid | 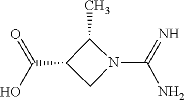 |
| (2S,3S)-1-carbamimido- yl-2-methylazetidine-3- carboxylic acid | 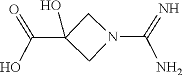 [SMILES: N=C(N)N1CC(O)(C(=O)O)C1] |
| 1-carbamimidoyl-3- hydroxyazetidine-3- carboxylic acid | 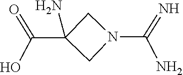 [SMILES: N=C(N)N1CC(N)(C(=O)O)C1] |
| 3-amino-1-carbamimido- ylazetidine-3-carboxylic acid | 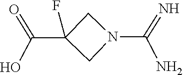 [SMILES: N=C(N)N1CC(F)(C(=O)O)C1] |
| 1-carbamimidoyl-3- fluoroazetidine-3- carboxylic acid | 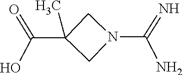 |
| 1-carbamimidoyl-3- methylazetidine-3- carboxylic acid | 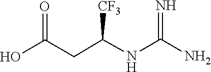 [SMILES: N=C(N)N[C@@H](CC(=O)O)C(F)(F)F] |
| (S)-3-guanidino-4,4,4- trifluorobutanoic acid | 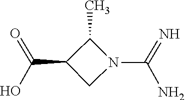 |
| (R)-3-guanidino-4,4,4- trifluorobutanoic acid | 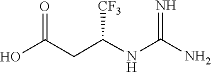 [SMILES: N=C(N)N[C@H](CC(=O)O)C(F)(F)F] |
| (2S,3S)-2-amino-3- carbamimidamido- 4,4,4-trifluorobutanoic acid | 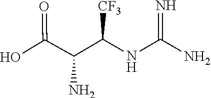 [SMILES: N=C(N)N[C@@H]([C@H](N)C(=O)O)C(F)(F)F] |
| (2S,3R)-2-amino-3- carbamimidamido- 4,4,4-trifluorobutanoic acid | 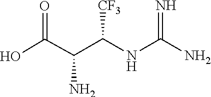 [SMILES: N=C(N)N[C@H]([C@H](N)C(=O)O)C(F)(F)F] |
| (2R,3R)-2-amino-3- carbamimidamido- 4,4,4-trifluorobutanoic acid | 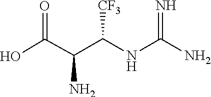 [SMILES: N=C(N)N[C@H]([C@@H](N)C(=O)O)C(F)(F)F] |
| (2R,3S)-2-amino-3- carbamimidamido- 4,4,4-trifluorobutanoic acid | 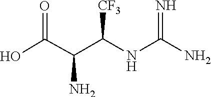 [SMILES: N=C(N)N[C@@H]([C@@H](N)C(=O)O)C(F)(F)F] |
| (3R)-3-carbamimid- amido(4,4,4-²H₃)- butanoic acid | 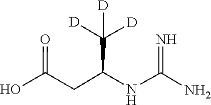 [SMILES: [2H]C([2H])([2H])[C@H](CC(=O)O)NC(=N)N] |
| (3S)-3-carbamimid- amido(4,4,4-²H₃)- butanoic acid | 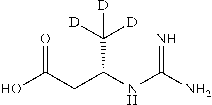 [SMILES: [2H]C([2H])([2H])[C@@H](CC(=O)O)NC(=N)N] |
| (3S)-3-carbamimid- amido-4,4-difluoro- butanoic acid | 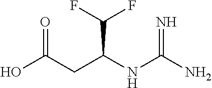 [SMILES: N=C(N)N[C@@H](CC(=O)O)C(F)F] |
| 3-carbamimidamido- 4-fluorobutanoic acid | 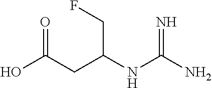 [SMILES: N=C(N)NC(CF)CC(=O)O] |
| (3S)-3-carbamimid- amidopent-4-enoic acid | 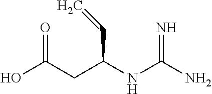 |
| (3R)-3-carbamimid- amidopent-4-enoic acid | 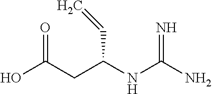 |
| (3S)-3-carbamimid- amidopent-4-ynoic acid | 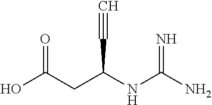 |
| (3R)-3-carbamimid- amidopent-4-ynoic acid | 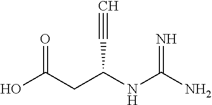 |
| 3-carbamimidamido- pentanoic acid | 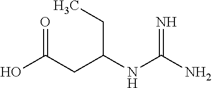 |
| (3R)-3-carbamimid- amidopentanoic acid | 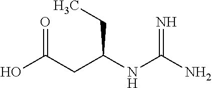 |
| (3R)-3-carbamimid- amidohexanoic acid | 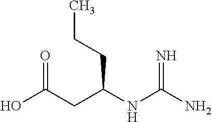 |
| (3S)-3-carbamimid- amidohexanoic acid | 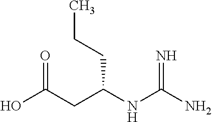 |
| 3-carbamimidamido- 4-methylpentanoic acid | 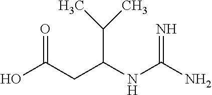 |
| (3S)-3-carbamimid- amido-4-methyl- pentanoic acid | 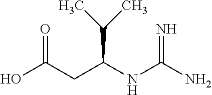 |
| (3R)-3-carbamimid- amido-4-methyl- pentanoic acid | 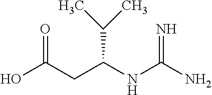 |
| 3-carbamimidamido- 2,2-dimethylpropanoic acid | 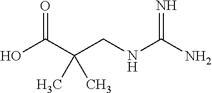 |
| 1-(carbamimidamido- methyl)cyclopropane- 1-carboxylic acid | 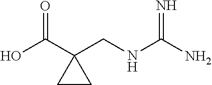 [SMILES: N=C(N)NCC1(C(=O)O)CC1] |
| 1-(carbamimidamido- methyl)cyclobutane- 1-carboxylic acid | 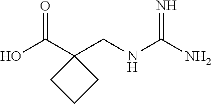 [SMILES: N=C(N)NCC1(C(=O)O)CCC1] |
| 3-carbamimidamido- 3-methylbutanoic acid | 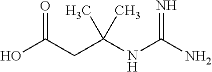 |
| 2-(1-carbamimid- amidocyclopropyl)- acetic acid | 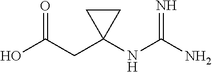 [SMILES: N=C(N)NC1(CC(=O)O)CC1] |
| 2-(1-carbamimid- amidocyclobutyl)- acetic acid | 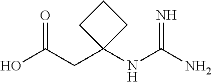 [SMILES: N=C(N)NC1(CC(=O)O)CCC1] |
| (2R,3R)-3-carbamimid- amido-2-methylbutanoic acid | 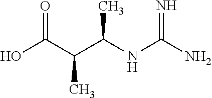 |
| (2S,3R)-3-carbamimid- amido-2-methylbutanoic acid | 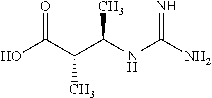 |
| (2R,3S)-3-carbamimid- amido-2-methylbutanoic acid | 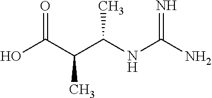 |
| (2S,3S)-3-carbamimid- amido-2-methylbutanoic acid | 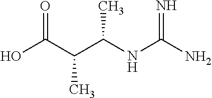 |
| 1-carbamimidoyl- pyrrolidine-3- carboxylic acid | 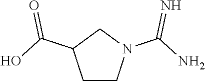 [SMILES: N=C(N)N1CCC(C(=O)O)C1] |
| (3S)-1-carbamimido- ylpyrrolidine-3- carboxylic acid | 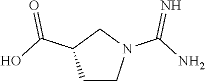 [SMILES: N=C(N)N1CC[C@H](C(=O)O)C1] |
| 3-carbamimidamido- 3-methylbutanoic acid | 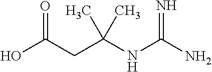 |
| 2-(1-carbamimid- amidocyclopropyl)- acetic acid | 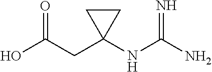 [SMILES: N=C(N)NC1(CC(=O)O)CC1] |
| 2-(1-carbamimid- amidocyclobutyl)- acetic acid | 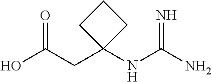 [SMILES: N=C(N)NC1(CC(=O)O)CCC1] |
| (2R,3R)-3-carbamimid- amido-2-methylbutanoic acid | 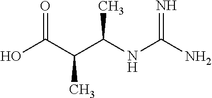 |
| (2S,3R)-3-carbamimid- amido-2-methylbutanoic acid | 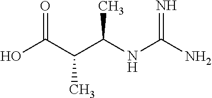 |
| (2R,3S)-3-carbamimid- amido-2-methylbutanoic acid | 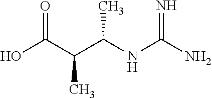 |
| (2S,3S)-3-carbamimid- amido-2-methylbutanoic acid | 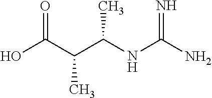 |
| (3R)-1-carbamimido- ylpyrrolidine-3- carboxylic acid | 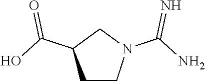 [SMILES: N=C(N)N1CC[C@@H](C(=O)O)C1] |
| 2-[(2R)-1-carbamimido- ylazetidin-2-yl]acetic acid | 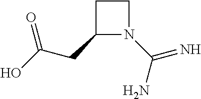 [SMILES: N=C(N)N1CC[C@@H]1CC(=O)O] |
| 2-[(2S)-1-carbamimido- ylazetidin-2-yl]acetic acid | 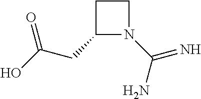 [SMILES: N=C(N)N1CC[C@H]1CC(=O)O] |
| cis-3-carbamimid- amidocyclobutane-1- carboxylic acid | 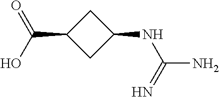 [SMILES: N=C(N)N[C@H]1C[C@@H](C(=O)O)C1] |
| trans-3-carbamimid- amidocyclobutane- 1-carboxylic acid | 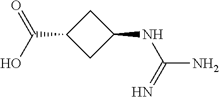 [SMILES: N=C(N)N[C@H]1C[C@H](C(=O)O)C1] |
| 3-carbamimidamido- 1-hydroxycyclobutane- 1-carboxylic acid | 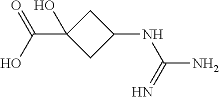 [SMILES: N=C(N)NC1CC(O)(C(=O)O)C1] |
| 1-amino-3-carbamimid- amidocyclobutane-1- carboxylic acid | 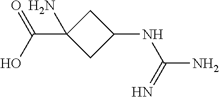 [SMILES: N=C(N)NC1CC(N)(C(=O)O)C1] |
| 2-(1-carbamimidoyl- azetidin-3-yl)acetic acid | 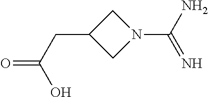 [SMILES: N=C(N)N1CC(CC(=O)O)C1] |
| (2S)-2-amino-3- [(pyridin-2-yl)amino]- propanoic acid | 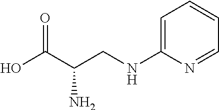 [SMILES: N[C@@H](CNc1ccccn1)C(=O)O] |
| (2S,3S)-2-amino-3- [(pyridin-2-yl)amino]- butanoic acid | 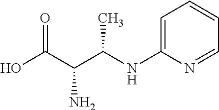 |
| (2S,3R)-2-amino-3- [(pyridin-2-yl)amino]- butanoic acid | 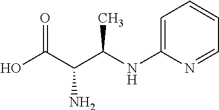 |
| cis-2-[(pyridin-2-yl)- amino]cyclopropane- 1-carboxylic acid | 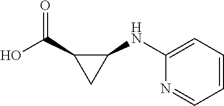 [SMILES: O=C(O)[C@@H]1C[C@@H]1Nc1ccccn1] |
| trans-2-[(pyridin-2-yl)- amino]cyclopropane- 1-carboxylic acid | 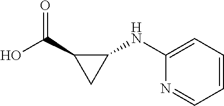 [SMILES: O=C(O)[C@@H]1C[C@H]1Nc1ccccn1] |
| 3-[(pyridin-2-yl)- amino](2H4)- propanoic acid | 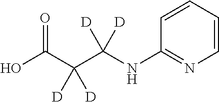 [SMILES: [2H]C([2H])(Nc1ccccn1)C([2H])([2H])C(=O)O] |
| 3-[(pyridin-2-yl)- amino](3,3-2H2)- propanoic acid | 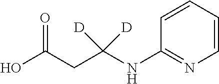 [SMILES: [2H]C([2H])(CC(=O)O)Nc1ccccn1] |
| 2,2-difluoro-3- [(pyridin-2-yl)- amino]propanoic acid | 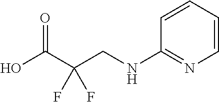 [SMILES: O=C(O)C(F)(F)CNc1ccccn1] |
| cis-2-[(pyridin-2-yl)- amino]cyclobutane-1- carboxylic acid | 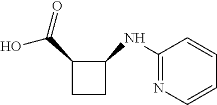 [SMILES: O=C(O)[C@@H]1CC[C@@H]1Nc1ccccn1] |
| trans-2-[(pyridin-2- yl)amino]cyclobutane- 1-carboxylic acid | 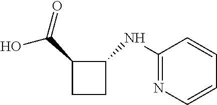 [SMILES: O=C(O)[C@@H]1CC[C@H]1Nc1ccccn1] |
| 4,4,4-trifluoro-3- [(pyridine-2-yl)- amino]butanoic acid | 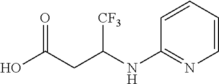 [SMILES: O=C(O)CC(Nc1ccccn1)C(F)(F)F] |
| 2-amino-4,4,4-trifluoro- 3-[(pyridine-2-yl)- amino]butanoic acid | 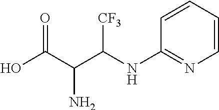 [SMILES: NC(C(=O)O)C(Nc1ccccn1)C(F)(F)F] |
| (2R)-2-amino-3- [(pyridin-2-yl)- amino]propanoic acid | 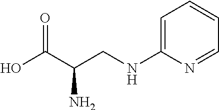 [SMILES: N[C@H](CNc1ccccn1)C(=O)O] |
| (2R,3S)-2-amino-3- [(pyridin-2-yl)amino]- butanoic acid | 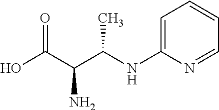 |
| (1R,2S)-2-[(pyridin-2- yl)amino]cyclopropane- 1-carboxylic acid | 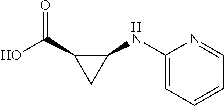 [SMILES: O=C(O)[C@@H]1C[C@@H]1Nc1ccccn1] |
| (1S,2R)-2-[(pyridin-2- yl)amino]cyclopropane- 1-carboxylic acid | 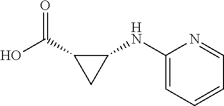 [SMILES: O=C(O)[C@H]1C[C@H]1Nc1ccccn1] |
| (1R,2R)-2-[(pyridin-2- yl)amino]cyclopropane- 1-carboxylic acid | 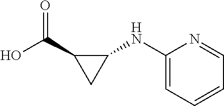 [SMILES: O=C(O)[C@@H]1C[C@H]1Nc1ccccn1] |
| (1S,2S)-2-[(pyridin-2- yl)amino]cyclopropane- 1-carboxylic acid | 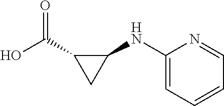 [SMILES: O=C(O)[C@H]1C[C@@H]1Nc1ccccn1] |
| (1R,2S)-2-[(pyridin-2- yl)amino]cyclobutane- 1-carboxylic acid | 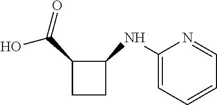 [SMILES: O=C(O)[C@@H]1CC[C@@H]1Nc1ccccn1] |
| (1S,2R)-2-[(pyridin-2- yl)amino]cyclobutane- 1-carboxylic acid | 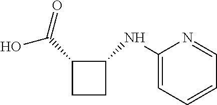 [SMILES: O=C(O)[C@H]1CC[C@H]1Nc1ccccn1] |
| (1R,2R)-2-[(pyridin-2- yl)amino]cyclobutane- 1-carboxylic acid | 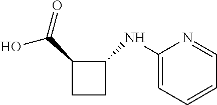 [SMILES: O=C(O)[C@@H]1CC[C@H]1Nc1ccccn1] |
| (1S,2S)-2-[(pyridin-2- yl)amino]cyclobutane- 1-carboxylic acid | 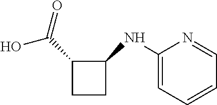 [SMILES: O=C(O)[C@H]1CC[C@@H]1Nc1ccccn1] |
| (2R,3R)-2-methyl-1- (pyridin-2-yl)azetidine- 3-carboxylic acid | 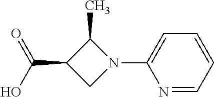 |
| (2R,3S)-2-methyl-1- (pyridin-2-yl)azetidine- 3-carboxylic acid | 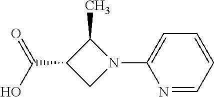 |
| (2S,3R)-2-methyl-1- (pyridin-2-yl)azetidine- 3-carboxylic acid | 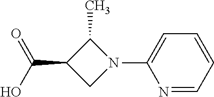 |
| (2S,3S)-2-methyl-1- (pyridin-2-yl)azetidine- 3-carboxylic acid | 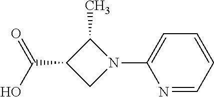 |
| 3-hydroxy-1-(pyridin- 2-yl)azetidine-3- carboxylic acid | 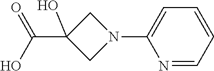 [SMILES: O=C(O)C1(O)CN(c2ccccn2)C1] |
| 3-amino-1-(pyridin-2- yl)azetidine-3-carbox- ylic acid | 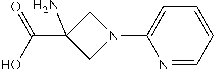 [SMILES: NC1(C(=O)O)CN(c2ccccn2)C1] |
| 3-fluoro-1-(pyridin-2- yl)azetidine-3-carbox- ylic acid | 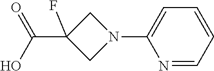 [SMILES: O=C(O)C1(F)CN(c2ccccn2)C1] |
| 3-methyl-1-(pyridin-2- yl)azetidine-3-carbox- ylic acid | 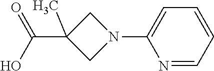 |
| (3S)-4,4,4-trifluoro-3- [(pyridin-2-yl)amino]- butanoic acid | 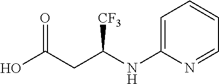 [SMILES: O=C(O)C[C@H](Nc1ccccn1)C(F)(F)F] |
| (3R)-4,4,4-trifluoro-3- [(pyridin-2-yl)amino]- butanoic acid | 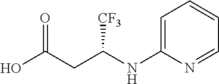 [SMILES: O=C(O)C[C@@H](Nc1ccccn1)C(F)(F)F] |
| (2S,3S)-2-amino-4,4,4- trifluoro-3-[(pyridin-2- yl)amino]butanoic acid | 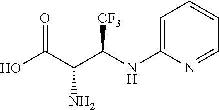 [SMILES: N[C@H](C(=O)O)[C@H](Nc1ccccn1)C(F)(F)F] |
| (2S,3R)-2-amino-4,4,4- trifluoro-3-[(pyridin-2- yl)amino]butanoic acid | 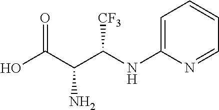 [SMILES: N[C@H](C(=O)O)[C@@H](Nc1ccccn1)C(F)(F)F] |
| (2R,3R)-2-amino-4,4,4- trifluoro-3-[(pyridin-2- yl)amino]butanoic acid | 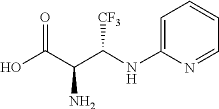 [SMILES: N[C@@H](C(=O)O)[C@@H](Nc1ccccn1)C(F)(F)F] |
| (2R,3S)-2-amino-4,4,4- trifluoro-3-[(pyridin-2- yl)amino]butanoic acid | 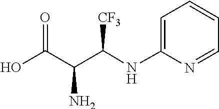 [SMILES: N[C@@H](C(=O)O)[C@H](Nc1ccccn1)C(F)(F)F] |
| (3R)-3-[(pyridin-2-yl)- amino](4,4,4-²H₃)- butanoic acid | 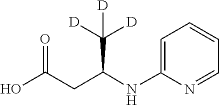 [SMILES: [2H]C([2H])([2H])[C@H](CC(=O)O)Nc1ccccn1] |
| (3S)-3-[(pyridin-2-yl)- amino](4,4,4-²H₃)- butanoic acid | 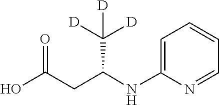 [SMILES: [2H]C([2H])([2H])[C@@H](CC(=O)O)Nc1ccccn1] |
| (3S)-4,4-difluoro-3- [(pyridin-2-yl)amino]- butanoic acid | 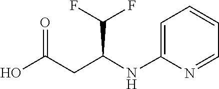 [SMILES: O=C(O)C[C@H](Nc1ccccn1)C(F)F] |
| (3R)-4,4-difluoro-3- [(pyridin-2-yl)amino]- butanoic acid | 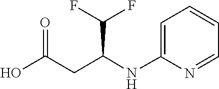 [SMILES: O=C(O)C[C@H](Nc1ccccn1)C(F)F] |
| 4-fluoro-3-[(pyridin-2- yl)amino]butanoic acid | 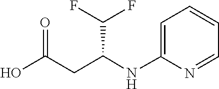 [SMILES: O=C(O)C[C@@H](Nc1ccccn1)C(F)F] |
| (3S)-3-[(pyridin-2-yl)- amino]pent-4-enoic acid | 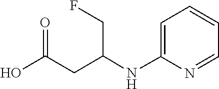 [SMILES: O=C(O)CC(CF)Nc1ccccn1] |
| (3R)-3-[(pyridin-2-yl)- amino]pent-4-enoic acid | 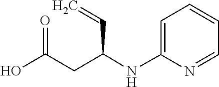 |
| (3S)-3-[(pyridin-2-yl)- amino]pent-4-ynoic acid | 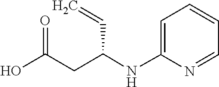 |
| (3R)-3-[(pyridin-2-yl)- amino]pent-4-ynoic acid | 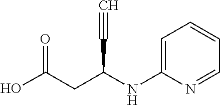 |
| (3R)-3-[(pyridin-2-yl)- amino]pentanoic acid | 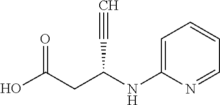 |
| (3S)-3-[(pyridin-2-yl)- amino]pentanoic acid | 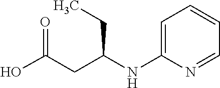 |
| 2,2-dimethyl-3-[(pyridin- 2-yl)amino]propanoic acid | 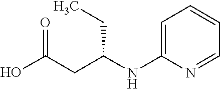 |
| 1-{[(pyridin-2-yl)amino]- methyl}cyclopropane-1- carboxylic acid | 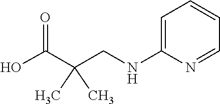 |
| 1-{[(pyridin-2-yl)amino]- methyl}cyclobutane-1- carboxylic acid | 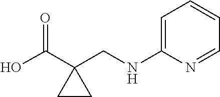 [SMILES: O=C(O)C1(CNc2ccccn2)CC1] |
| 3-methyl-3-[(pyridin-2- yl)amino]butanoic acid | 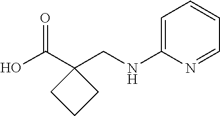 [SMILES: O=C(O)C1(CNc2ccccn2)CCC1] |
| 2-{1-[(pyridin-2-yl)- amino]cyclopropyl}- acetic acid | 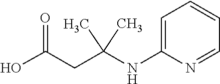 |
| 2-{1-[(pyridin-2-yl)- amino]cyclobutyl}- acetic acid | 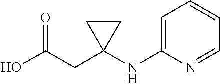 [SMILES: O=C(O)CC1(Nc2ccccn2)CC1] |
| (2R,3R)-2-methyl-3- [(pyridin-2-yl)amino- ]butanoic acid | 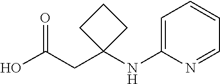 [SMILES: O=C(O)CC1(Nc2ccccn2)CCC1] |
| (2S,3R)-2-methyl-3- [(pyridin-2-yl)amino]- butanoic acid | 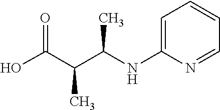 |
| (2R,3S)-2-methyl-3- [(pyridin-2-yl)amino]- butanoic acid | 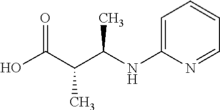 |
| (2R)-2-amino-3- (carbamimidoylsulfan- yl)propanoic acid | 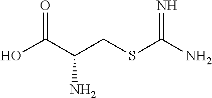 |
| (2S,3S)-2-amino-3- (carbamimidoylsulfan- yl)butanoic acid | 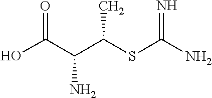 |
| (2S,3R)-2-amino-3- (carbamimidoylsulfan- yl)butanoic acid | 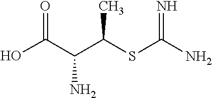 |
| cis-2-(carbamimidoyl- sulfanyl)cyclopropane- 1-carboxylic acid | 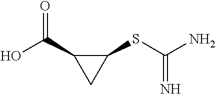 |
| trans-2-(carbamimido- ylsulfanyl)cyclopropane- 1-carboxylic acid | 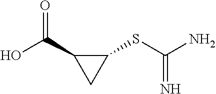 |
| 3-(carbamimidoyl- sulfanyl)(2,2-2H4)- propanoic acid | 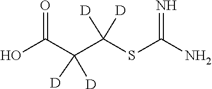 [SMILES: [2H]C([2H])(SC(=N)N)C([2H])([2H])C(=O)O] |
| 3-(carbamimidoyl- sulfanyl)(2,2-2H)- propanoic acid | 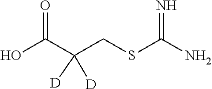 [SMILES: [2H]C([2H])(CSC(=N)N)C(=O)O] |
| 3-(carbamimidoyl- sulfanyl)-2,2-difluoro- propanoic acid | 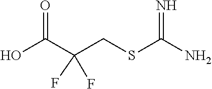 [SMILES: N=C(N)SCC(F)(F)C(=O)O] |
| cis-2-(carbamimidoyl- sulfanyl)cyclobutane- 1-carboxylic acid | 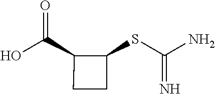 [SMILES: N=C(N)S[C@H]1CC[C@H]1C(=O)O] |
| trans-2-(carbamimidoyl- sulfanyl)cyclobutane-1- carboxylic acid | 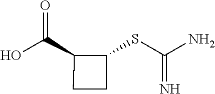 [SMILES: N=C(N)S[C@@H]1CC[C@H]1C(=O)O] |
| 3-(carbamimidoyl- sulfanyl)-4,4,4-trifluoro- butanoic acid | 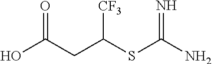 [SMILES: N=C(N)SC(CC(=O)O)C(F)(F)F] |
| 2-amino-3-(carbam- imidoylsulfanyl)-4,4,4- trifluorobutanoic acid | 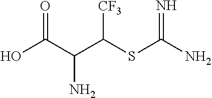 [SMILES: N=C(N)SC(C(N)C(=O)O)C(F)(F)F] |
| (3R)-3-carbamimid- amidobutanoic acid | 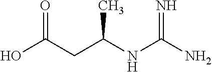 |
| (3S)-3-carbamimid- amidobutanoic acid | 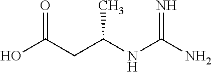 |
| (2S,3R)-2-amino-3- carbamimidamido- butanoic acid | 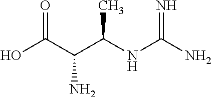 |
| (2S,3S)-2-amino-3- carbamimidamido- butanoic acid | 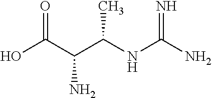 |
| (2R,3R)-2-amino-3- carbamimidamido- butanoic acid | 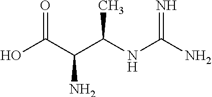 |
| (2R,3S)-2-amino-3- carbamimidamido- butanoic acid | 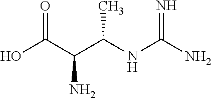 |
| (2S)-1-carbamimidoyl- azetidine-2-carboxylic acid | 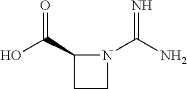 |
| (2R)-1-carbamimidoyl- azetidine-2-carboxylic acid | 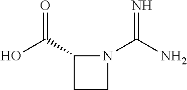 |
| (3R)-3-[(pyridin-2-yl)- amino]butanoic acid | 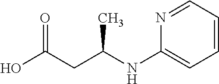 |
| (3S)-3-[(pyridin-2-yl)- amino]butanoic acid | 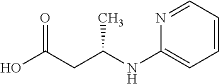 |
| (3R)-3-[(pyridin-2-yl)- amino]hexanoic acid | 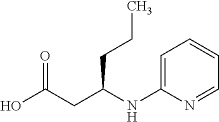 |
| (3S)-4-methyl-3- [(pyridin-2-yl)amino]- hexanoic acid | 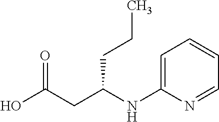 |
| (3S)-4-methyl-3- [(pyridin-2-yl)amino]- pentanoic acid | 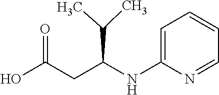 |
| (3R)-4-methyl-3- [(pyridin-2-yl)amino]- pentanoic acid | 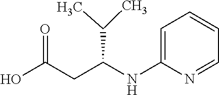 |
| (3S)-1-(pyridin-2-yl)- pyrrolidine-3-carboxylic acid | 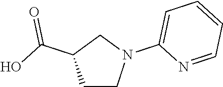 [SMILES: O=C(O)[C@H]1CCN(c2ccccn2)C1] |
| (3R)-1-(pyridin-2-yl)- pyrrolidine-3-carboxylic acid | 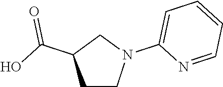 [SMILES: O=C(O)[C@@H]1CCN(c2ccccn2)C1] |
| (1-methylguanidino- methyl)phosphinic acid | 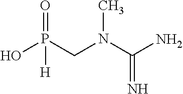 |
| [(2-iminoimidazolidin- 1-yl)methyl]phosphinic acid | 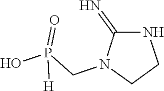 |
| [(2-imino-3-(phos- phono)imidazolidin-1- yl)methyl]phosphinic acid | 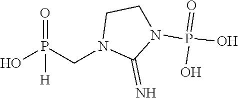 [SMILES: N=C1N(C[PH](=O)O)CCN1P(=O)(O)O] |
| {1-methyl-3-(phos- phono)guanidino- methyl}phosphinic acid | 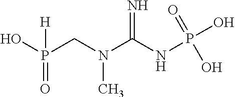 |
| 1-(N-phosphono- carbamimidoyl)- azetidine-2- carboxylic acid | 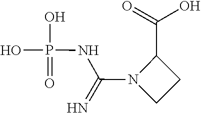 [SMILES: N=C(NP(=O)(O)O)N1CCC1C(=O)O] |
| 1-(N-phosphono- carbamimidoyl)- pyrrolidine-2- carboxylic acid | 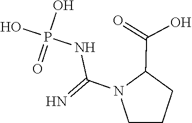 [SMILES: N=C(NP(=O)(O)O)N1CCCC1C(=O)O] |
| 1-carbamimidoyl- azetidine-2- carboxylic acid | 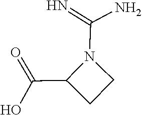 |
| 1-carbamimidoyl- pyrrolidine-2- carboxylic acid | 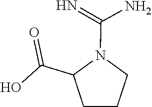 [SMILES: N=C(N)N1CCCC1C(=O)O] |
| 2-(1-ethylguanidino)- acetic acid | 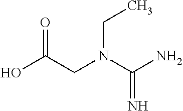 |
| 2-(1-methylguanidino)- acetic acid (Creatine) | 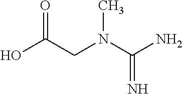 |
| 2-(1-methylguanidino)- propanoic acid | 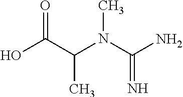 |
| 2-(3-phosphono-1- propylguanidino)acetic acid | 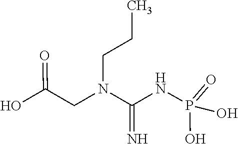 |
| 2-(1-phosphonocarbam- imidamido)acetic acid | 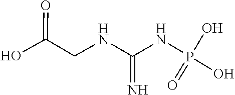 [SMILES: N=C(NCC(=O)O)NP(=O)(O)O] |
| 2-(1-propylguanidino)- acetic acid | 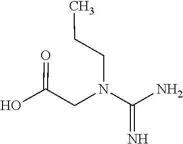 |
| 2-(2-amino-1H-imidazol- 1-yl)acetic acid | 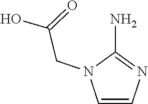 |
| 2-(2-imino-1,3- diazinan-1-yl)acetic acid (1-Carboxymethyl- 2-iminohexahydro- pyrimidine) | 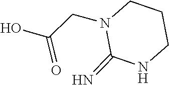 [SMILES: N=C1NCCCN1CC(=O)O] |
| 2-(2-imino-3-phos- phonoimidazolidin-1- yl)acetic acid (N- Phosphoryl Cyclo- creatine) | 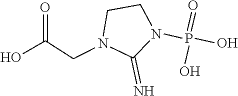 [SMILES: N=C1N(CC(=O)O)CCN1P(=O)(O)O] |
| 2-(2-iminoimidazolidin- 1-yl)acetic acid (Cyclo- creatine) | 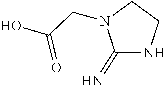 |
| 2-(3-phosphono-1-ethyl- guanidino)acetic acid | 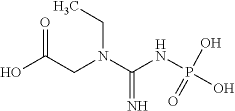 |
| 2-(3-phosphono-1- methylguanidino)acetic acid (N-Phosphoryl Creatine) | 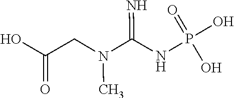 |
| 2-(3-phosphono-1- methylguanidino)- propionic acid | 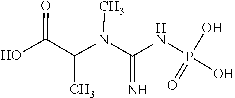 |
| 2-carbamimidamido- acetic acid (Guanidino- acetic acid) | 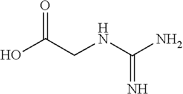 |
| 3-(1-methylguanidino)- propanoic acid (Homo- creatine) | 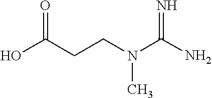 |
| 3-(3-phosphono- guanidino)propionic acid | 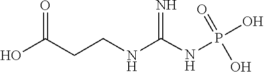 [SMILES: N=C(NCCC(=O)O)NP(=O)(O)O] |
| 3-(2-imino-3-phos- phonoimidazolidin-1- yl)propanoic acid (N- Phosphoryl Homocyclo- creatine) | 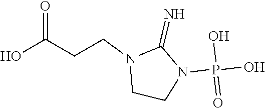 [SMILES: N=C1N(CCC(=O)O)CCN1P(=O)(O)O] |
| 3-(2-iminoimidazolidin- 1-yl)propanoic acid (Homocyclocreatine) | 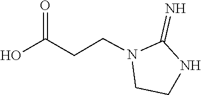 [SMILES: N=C1NCCN1CCC(=O)O] |
| 3-(3-phosphono-1- methylguanidino)- propionic acid (Phosohomocreatine) | 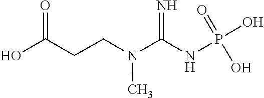 |
| 3-carbamimidamido- propanoic acid (β- Guanidinopropionic acid; β-GPA) | 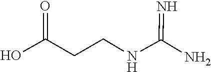 |
| 3-carbamimidoyl-3- methylpropanoic acid (Carbocreatine) | 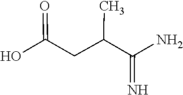 |
| 2-(1,3-dimethyl- guanidino)acetic acid | 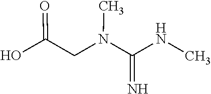 |
| 2-carbamimidamido- butanedioic acid | 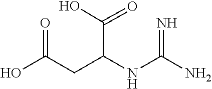 [SMILES: N=C(N)NC(CC(=O)O)C(=O)O] |
| 2-carbamimidamido- butanoic acid | 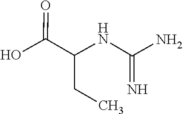 |
| 2-carbamimidamido- propanoic acid | 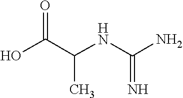 |
| 3-carbamimidamido- butanoic acid(β-DL- Guanidinobutanoicic acid; β-GBA) | 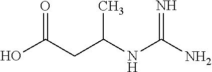 |
| 4-carbamimidamido- butanoic acid | 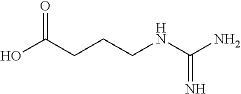 |
| 2-({[N′-hydroxy- carbamimidoy]]- methyl}amino)- acetic acid | 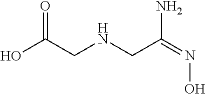 |
| 2-({3-[(carboxy- methylidene)amino]- guanidino}imino)- acetic acid | 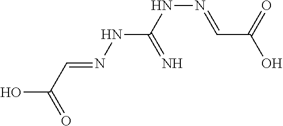 [SMILES: N=C(N/N=C/C(=O)O)N/N=C/C(=O)O] |
| 2-({bis[(2-aminoethyl)- amino]methylidene}- amino)acetic acid | 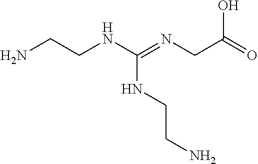 [SMILES: NCCNC(=NCC(=O)O)NCCN] |
| 2-({bis[(2-hydroxy- ethyl)amino]methyl- idene}amino)acetic acid | 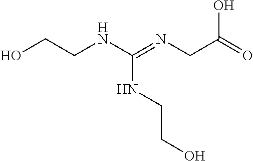 [SMILES: O=C(O)CN=C(NCCO)NCCO] |
| 2-({bis[(3,5-dimethyl- 1H-pyrazol-1-yl)- amino methylidene}- amino)acetic acid | 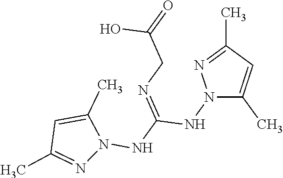 |
| 2-({bis[(carbamoyl- amino)amino]methyl- idene}amino)acetic acid | 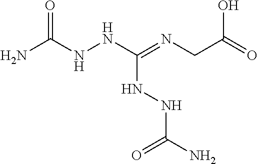 [SMILES: NC(=O)NNC(=NCC(=O)O)NNC(N)=O] |
| 2-(1-aminoguanidino)- acetic acid | 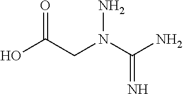 |
| 2-(1,3-diamino- guanidino)acetic acid | 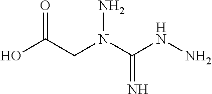 |
| 2-{N-(2-amino)ethan- imidamido}acetic acid | 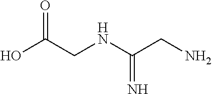 |
| 2-(carbamimidamido- amino)-2-phenylacetic acid | 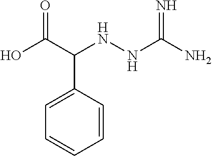 [SMILES: N=C(N)NNC(C(=O)O)c1ccccc1] |
| 2-(carbamimidamido- amino)-3-phenylpro- panoic acid | 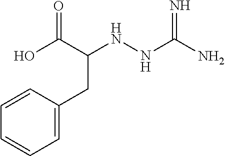 [SMILES: N=C(N)NNC(Cc1ccccc1)C(=O)O] |
| 2-(carbamimidamido- amino)acetic acid | 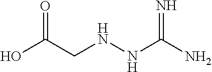 |
| 2-(carbamimidamido- amino)butanoic acid | 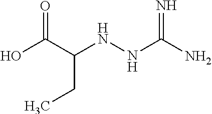 |
| 2-(carbamimidamido- imino)-2-phenylacetic acid | 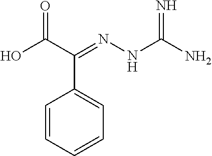 [SMILES: N=C(N)N/N=C(/C(=O)O)c1ccccc1] |
| 2-(carbamimidamido- imino)-3-phenylpro- panoic acid | 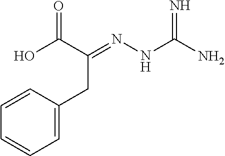 [SMILES: N=C(N)N/N=C(\Cc1ccccc1)C(=O)O] |
| 2-(carbamimidamido- imino)acetic acid | 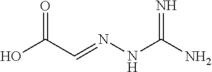 |
| 2-(carbamimidamido- imino)butanoic acid | 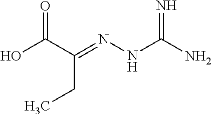 |
| 2-(carbamimidamido- imino)octanoic acid | 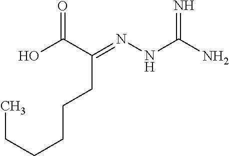 |
| 2-(carbamimidamido- imino)propanoic acid | 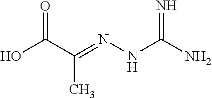 |
| 2-(N-carbamimidoyl- imidamido)acetic acid | 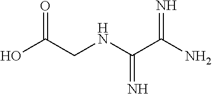 |
| 2-[(1-methylguanidino)- imino]acetic acid | 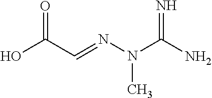 |
| 2-[(2-amino-1H-imid- azol-1-yl)amino]acetic acid | 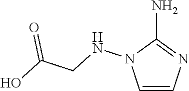 [SMILES: Nc1nccn1NCC(=O)O] |
| 2-[(2,10-dimethyl- 2,5,7,10-tetraazaun- decan-6-ylidene)- amino]acetic acid | 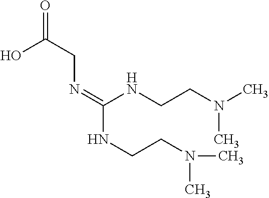 [SMILES: CN(C)CCNC(=NCC(=O)O)NCCN(C)C] |
| 2-[(3-amino-4H-1,2,4- triazol-4-yl)amino]acetic acid | 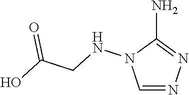 [SMILES: Nc1nncn1NCC(=O)O] |
| 2-[(6-oxo-1,2,4,5- tetrazinan-3-ylidene)- amino]acetic acid | 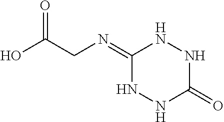 [SMILES: O=C(O)CN=C1NNC(=O)NN1] |
| 2-(biguanide)acetic acid | 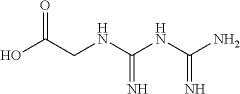 [SMILES: N=C(N)NC(=N)NCC(=O)O] |
| 2-(biguanide)ethane-1- sulfonic acid | 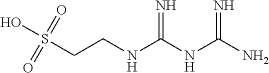 [SMILES: N=C(N)NC(=N)NCCS(=O)(=O)O] |
| 2-[(carbamimidoyl- methyl)amino]acetic acid | 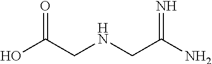 |
| 2-[(di{[(methoxy- carbonyl)amino]- amino}methylidene)- amino]acetic acid | 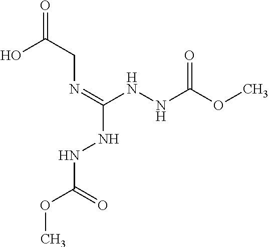 [SMILES: COC(=O)NNC(=NCC(=O)O)NNC(=O)OC] |
| 2-[(di{[(acetyl)amino]- aminomethylidene)- amino]acetic acid | 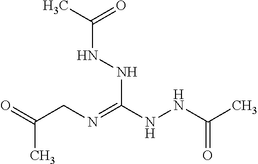 [SMILES: CC(=O)CN=C(NNC(C)=O)NNC(C)=O] |
| 2-[(dihydrazinylmeth- ylidene)amino]-2- phenylacetic acid | 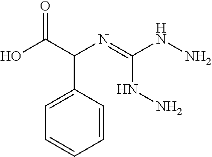 [SMILES: NNC(=NC(C(=O)O)c1ccccc1)NN] |
| 2-[(dihydrazinylmeth- ylidene)amino]-3- methylbutanoic acid | 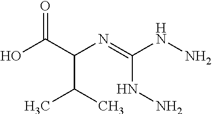 |
| 2-[(dihydrazinylmeth- ylidene)amino]-3- phenylpropanoic acid | 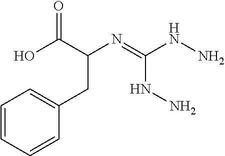 [SMILES: NNC(=NC(Cc1ccccc1)C(=O)O)NN] |
| 2-[(dihydrazinylmeth- ylidene)amino]acetic acid | 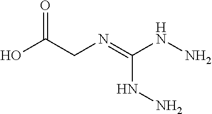 |
| 2-[(dihydrazinyl- methylidene)amino]- propanoic acid | 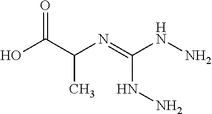 |
| 2-[[(2,2-dimethyl- hydrazin-1-yl)- (hydrazinyl)methyl- idene]amino]acetic acid | 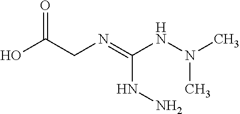 |
| 2-[{[(carbamoyl- amino)amino](hydra- zinyl)methylidene}- amino]acetic acid | 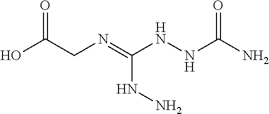 [SMILES: NN/C(=N\CC(=O)O)NNC(N)=O] |
| 2-[{hydraziny][(1H- pyrazol-5-yl)amino]- methylidene}amino]- acetic acid | 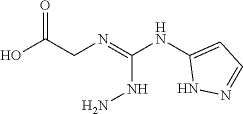 [SMILES: NN/C(=N\CC(=O)O)Nc1ccn[nH]1] |
| 2-[{hydraziny][(2- hydroxyethyl)amino- methylidene}amino]- acetic acid | 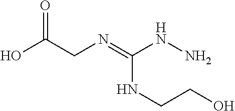 [SMILES: NN/C(=N/CC(=O)O)NCCO] |
| 2-[{hydraziny][(morph- olin-4-yl)amino]meth- ylidene}amino]acetic acid | 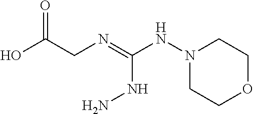 [SMILES: NN/C(=N\CC(=O)O)NN1CCOCC1] |
| 2-[{hydraziny][2- (pyridin-2-yl)hydrazin- 1-yl]methylidene}- amino]acetic acid | 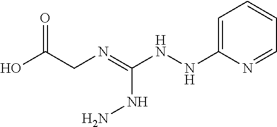 [SMILES: NN/C(=N\CC(=O)O)NNc1ccccn1] |
| 2-[2-(2-aminoethyl)- carbamimidamido]- acetic acid | 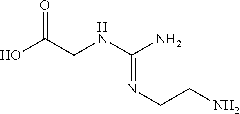 [SMILES: NCC/N=C(\N)NCC(=O)O] |
| 2-[2-(4,5-dihydro-1H- imidazol-2-yl)hydrazin- 1-ylidene]acetic acid | 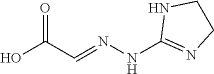 [SMILES: O=C(O)/C=N/NC1=NCCN1] |
| 2-[2-(pyridin-2-yl)- hydrazin-1-ylidene]- acetic acid | 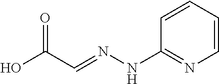 [SMILES: O=C(O)/C=N/Nc1ccccn1] |
| 2-[2-aminocarbam- imidamido]acetic acid | 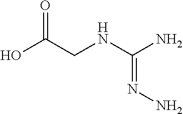 |
| 2-[2-hydroxycarbam- imidamido]acetic acid | 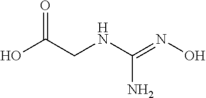 |
| 2-{[2-[(phenylmeth- ylidene)amino]carbam- imidamido]amino}- acetic acid | 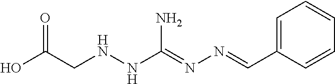 [SMILES: N/C(=N\N=C\c1ccccc1)NNCC(=O)O] |
| 2-{[2-[(propan-2-yl)- amino]carbamimid- amido]amino}acetic acid | 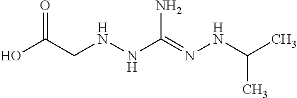 |
| 2-{[2-[(propan-2-yl- idene)amino]carbam- imidamido]amino}- acetic acid | 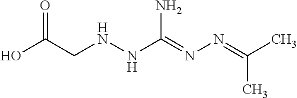 |
| 2-{[2-aminocarbam- imidamido]amino}- acetic acid | 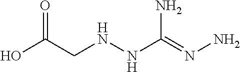 |
| 2-{[2-methylcarbam- imidamido]imino}- acetic acid | 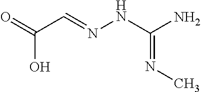 |
| 2-{[2-nitrocarbam- imidamido]imino}- acetic acid | 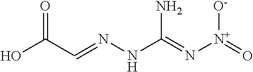 [SMILES: N/C(=N\[N+](=O)[O-])N/N=C/C(=O)O] |
| 2-{[4-amino-1,2,4- triazinan-3-ylidene]- amino}acetic acid | 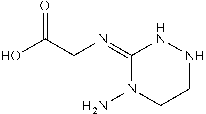 [SMILES: NN1CCNN/C1=N/CC(=O)O] |
| 2-{[6-methyl-1,2,3,4- tetrahydro-1,2,4,5- tetrazin-3-ylidene]- amino}acetic acid | 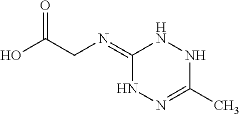 |
| 3-({[N′-hydroxy- carbamimidoyl]- methyl}amino)- propanoic acid | 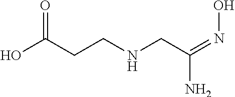 [SMILES: N/C(CNCCC(=O)O)=N/O] |
| 3-{N-(2-amino)- ethanimidamido}- propanoic acid | 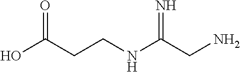 |
| 3-(carbamimidamido- amino)propanoic acid | 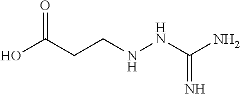 |
| 3-(N-carbamimidoyl- imidamido)propanoic acid | 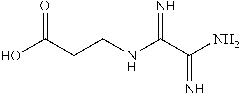 [SMILES: N=C(N)C(=N)NCCC(=O)O] |
| 3-(biguanide)- propanoic acid | 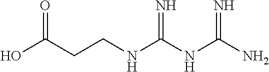 [SMILES: N=C(N)NC(=N)NCCC(=O)O] |
| 3-[(carbamimidoyl- methyl)amino]- propanoic acid | 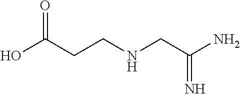 |
| 3-[(dihydrazinyl- methylidene)amino]- propanoic acid | 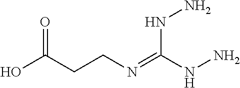 [SMILES: NNC(=NCCC(=O)O)NN] |
| 3-[2-aminocarbam- imidamido]propanoic acid | 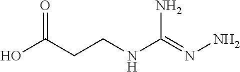 |
| 3-hydrazinylidene- 1,2,4-triazinan-5- one | 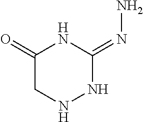 |
| 3-hydrazinylidene- 2-methyl-1,2,4- triazinan-6-one | 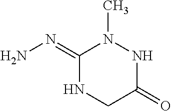 |
| 3-hydrazinylidene- 2,3,4,5-tetrahydro- 1,2,4-triazin-5-one | 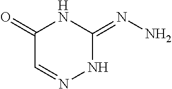 |
| 3-imino-1,2,4- triazinan-5-one | 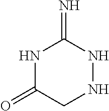 |
| 3-imino-2,3,4,5- tetrahydro-1,2,4- triazin-5-one | 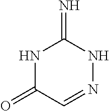 |
| 4-(biguanide)- butanoic acid | 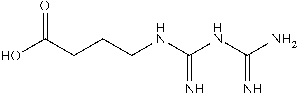 [SMILES: N=C(N)NC(=N)NCCCC(=O)O] |
| 4-[(dihydrazinyl- methylidene)- amino]butanoic acid | 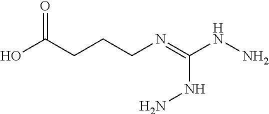 [SMILES: NNC(=NCCCC(=O)O)NN] |
| 4-[2-aminocarbam- imidamido]butanoic acid | 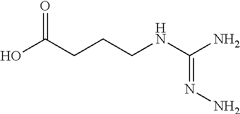 [SMILES: N/N=C(\N)NCCCC(=O)O] |
| ethyl 3-(biguanide)- propanoate | 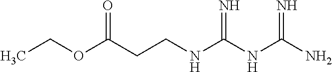 |
| methyl 2-(carbam- imidamidoamino)- acetate | 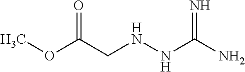 |
| methyl 2-(carbam- imidamidoimino)- acetate | 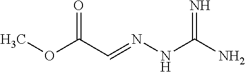 |
| methyl 3-(N-carbam- imidoylimidamido)- propanoate | 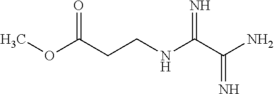 |
| methoxy(1-methyl- carbamimidamido- methyl)phosphinic acid | 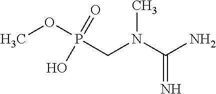 |
| 2-(1H-imidazol-2- yl)acetic acid | 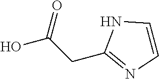 |
| 2-[1-amino-2-meth- ylguanidino]acetic acid | 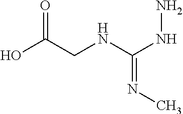 |
| 3-(1,4,5,6-tetrahydro- pyrimidin-2-yl)pro- panoic acid | 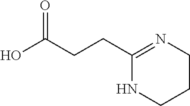 [SMILES: O=C(O)CCC1=NCCCN1] |
| 3-(1H-imidazol-2-yl)- propanoic acid | 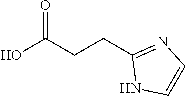 |
| 3-(4,5-dihydro-1H- imidazol-2-yl)pro- panoic acid | 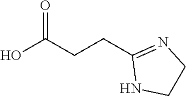 |
| 3-(4H-1,2,4-triazol- 3-yl)propanoic acid | 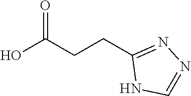 |
| 3-[(3,4-dihydro-2H- pyrrol-5-yl)amino]- propanoic acid | 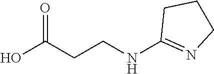 [SMILES: O=C(O)CCNC1=NCCC1] |
| (1R,2S)-2-carbam- imidamidocyclo- hexane-1-carboxylic acid | 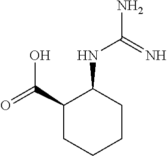 [SMILES: N=C(N)N[C@H]1CCCC[C@H]1C(=O)O] |
| (1R,2S)-2-carbam- imidamidocyclo- pentane-1-carbox- ylic acid | 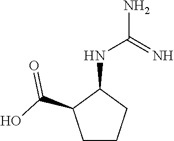 [SMILES: N=C(N)N[C@H]1CCC[C@H]1C(=O)O] |
| (1S,2S)-2-carbam- imidamidocyclo- hexane-1-carbox- ylic acid | 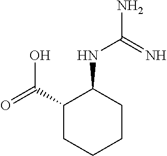 [SMILES: N=C(N)N[C@H]1CCCC[C@@H]1C(=O)O] |
| (1S,2S)-2-carbam- imidamidocyclo- pentane-1-carbox- ylic acid | 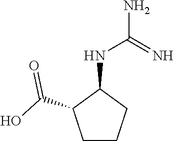 [SMILES: N=C(N)N[C@H]1CCC[C@@H]1C(=O)O] |
| (2-carbamimid- amidoethyl)phos- phonic acid | 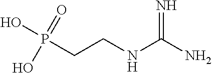 |
| (2R)-2-amino-3- carbamimidamido- propanoic acid | 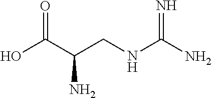 |
| (2S)-2-amino-3- carbamimidamido- propanoic acid | 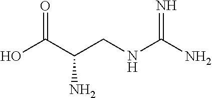 |
| (2S)-2-amino-5- carbamimidamido- pentanoic acid (L-Arg) | 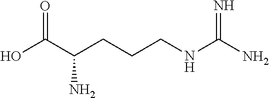 [SMILES: N=C(N)NCCC[C@H](N)C(=O)O] |
| 1-[2-(1H-1,2,3,4- tetrazol-5-yl)ethyl]- guanidine | 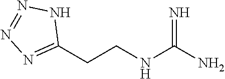 [SMILES: N=C(N)NCCc1nnn[nH]1] |
| 3-[(4,5-dihydro-1H- imidazol-2-yl)amino]- propanoic acid | 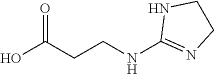 [SMILES: O=C(O)CCNC1=NCCN1] |
| 1-[2-(2-sulfanyl-1H- imidazol-5-yl)ethyl]- guanidine | 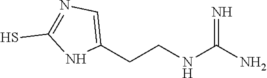 [SMILES: N=C(N)NCCc1cnc(S)[nH]1] |
| 1-carbamimidoyl- piperidine-3-carbox- ylic acid | 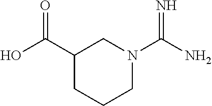 [SMILES: N=C(N)N1CCCC(C(=O)O)C1] |
| 2-(1-carbamimidoyl- piperidin-2-yl)acetic acid | 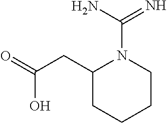 [SMILES: N=C(N)N1CCCCC1CC(=O)O] |
| 2-(1H-imidazol-4-yl)- acetic acid | 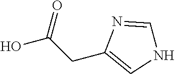 |
| 2-(5-carbamimidoyl- 1H-pyrrol-2-yl)acetic acid | 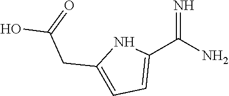 [SMILES: N=C(N)c1ccc(CC(=O)O)[nH]1] |
| 2-(6-aminopyridin-2- yl)acetic acid | 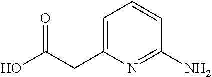 [SMILES: Nc1cccc(CC(=O)O)n1] |
| 2-(carbamimidamido- methyl)heptanoic acid | 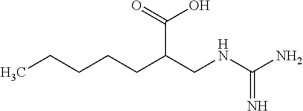 |
| 2-(carbamimidamido- oxy)acetic acid | 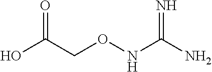 |
| 2-(carbamimidoyl- sulfanyl)acetic acid | 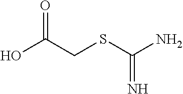 |
| 2-[(carbamimidoyl- methyl)sulfanyl]- acetic acid | 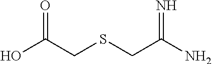 |
| 2-amino-1-(2-carbox- ylatoethyl)pyridin-1- ium | 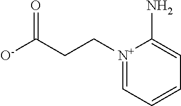 [SMILES: Nc1cccc[n+]1CCC(=O)[O-]] |
| 2-amino-3-(1H- imidazol-5-yl)- propanoic acid | 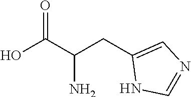 [SMILES: NC(Cc1cnc[nH]1)C(=O)O] |
| 2-phenyl-3-carbam- imidamidopropanoic acid | 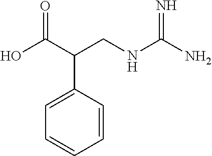 [SMILES: N=C(N)NCC(C(=O)O)c1ccccc1] |
| 2-carbamimidamido- ethane-1-sulfonic acid | 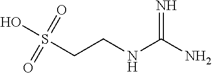 |
| 3-(3-hexylguanidino)- propanoic acid | 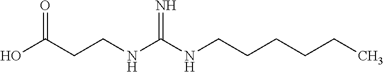 |
| 3-(3-methylguanidino)- propanoic acid | 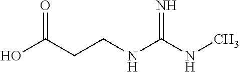 |
| 3-(1H-imidazol-1-yl)- propanoic acid | 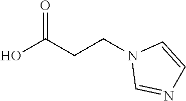 |
| 3-(2-carbamimidoyl- 1H-pyrrol-1-yl)- propanoic acid | 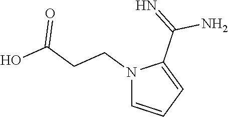 [SMILES: N=C(N)c1cccn1CCC(=O)O] |
| (E)-3-(carbamimidoyl- sulfanyl)prop-2-enoic acid | 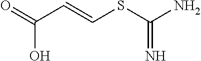 |
| (Z)-3-(carbamimidoyl- sulfanyl)prop-2-enoic acid | 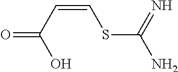 |
| 3-(carbamimidoyl- sulfanyl)propanoic acid | 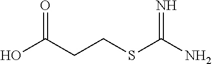 |
| 3-(carbamothioyl- amino)propanoic acid | 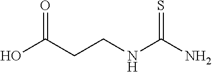 |
| 3-(carbamoylamino)- propanoic acid | 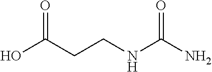 |
| 3-[(1,3-benzothiazol- 2-yl)amino]propanoic acid | 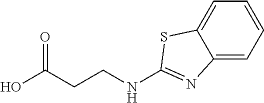 [SMILES: O=C(O)CCNc1nc2ccccc2s1] |
| 3-[(1,3-thiazol-2-yl)- amino]propanoic acid | 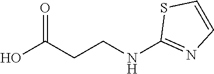 [SMILES: O=C(O)CCNc1nccs1] |
| 3-[(1H-1,3-benzo- diazol-2-yl)amino]- propanoic acid | 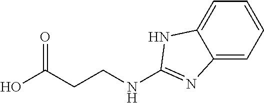 [SMILES: O=C(O)CCNc1nc2ccccc2[nH]1] |
| 3-[N-(2-aminobenzene- 1-carbamimido)]- propanoic acid | 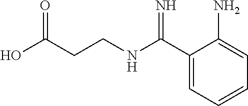 [SMILES: N=C(NCCC(=O)O)c1ccccc1N] |
| 3-[(2-carbamimidoyl- phenyl)amino]propanoic acid | 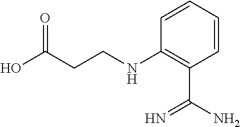 [SMILES: N=C(N)c1ccccc1NCCC(=O)O] |
| 3-[(4,5-dihydro-1,3- thiazol-2-yl)amino]- propanoic acid | 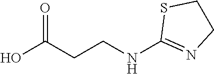 [SMILES: O=C(O)CCNC1=NCCS1] |
| 3-[(6-ethyl-4-oxo-1,4- dihydropyrimidin-2- yl)amino]propanoic acid | 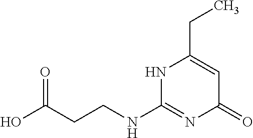 |
| 3-[(9H-purin-6-yl)- amino]propanoic acid | 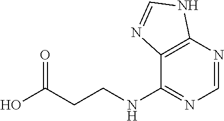 [SMILES: O=C(O)CCNc1ncnc2[nH]cnc12] |
| 3-[(N-methylcarbam- imidoyl)sulfanyl]- propanoic acid | 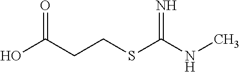 |
| 3-[(N,N-dimethyl- carbamimidoyl)- sulfanyl]propanoic acid | 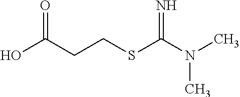 |
| 3-[(pyridin-2-yl)- amino]propanoic acid | 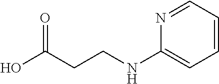 [SMILES: O=C(O)CCNc1ccccn1] |
| 3-[(pyrimidin-2-yl)- amino]propanoic acid | 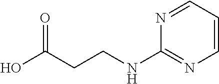 [SMILES: O=C(O)CCNc1ncccn1] |
| 3-[2-cyanocarbam- imidamido]propanoic acid | 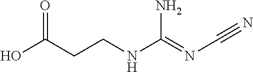 [SMILES: N#C/N=C(\N)NCCC(=O)O] |
| 3-[2-nitrocarbam- imidamido]propanoic acid | 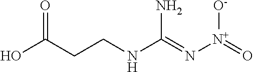 [SMILES: N/C(=N\[N+](=O)[O-])NCCC(=O)O] |
| 3-{[(acetylimino)- (amino)methyl]amino}- propanoic acid | 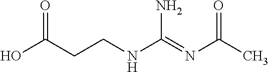 |
| 3-{[(methylsulfanyl)- methanimidoyl]amino}- propanoic acid | 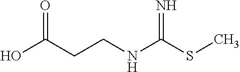 |
| 3-{[amino[(ethoxy- carbonyl)imino]- methyl]amino}- propanoic acid | 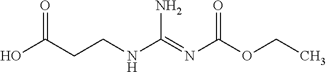 |
| 3-carbamimidamido- 2-(hydroxyimino)- propanoic acid | 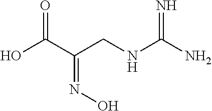 [SMILES: N=C(N)NC/C(=N\O)C(=O)O] |
| 3-carbamimidamido- 2-(methylamino)- propanoic acid | 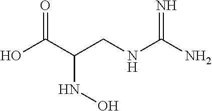 [SMILES: N=C(N)NCC(NO)C(=O)O] |
| 3-carbamimidamido- 2-hydroxypropanoic acid | 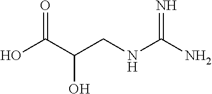 |
| 3-carbamimidamido- 2-methylpropanoic acid | 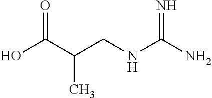 |
| 3-carbamimidamido- 2-sulfanylpropanoic acid | 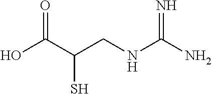 |
| 3-carbamimidamido- 3-phenylpropanoic acid | 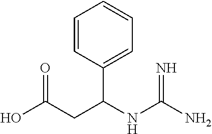 [SMILES: N=C(N)NC(CC(=O)O)c1ccccc1] |
| 3-carbamimidamido- N-hydroxypropan- amide | 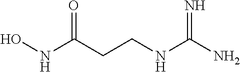 |
| 3-carbamimidamido- octanoic acid | 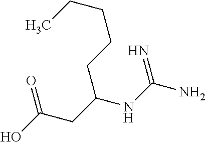 |
| 3-carbamimidamido- propanamide | 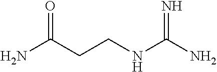 |
| 3-ethanimidamido- propanoic acid | 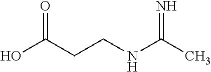 |
| 4-(carbamimidoyl- sulfanyl)butanoic acid | 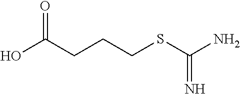 |
| 4-carbamimidamido- benzoic acid | 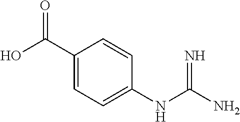 [SMILES: N=C(N)Nc1ccc(C(=O)O)cc1] |
| 4-carbamimidoyl- butanoic acid | 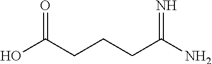 |
| ethyl 3-guanidino- propanoate | 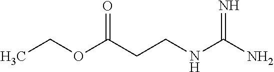 |
| 2-(2-imino-4-methyl- midazolidin-1-yl)acetic acid | 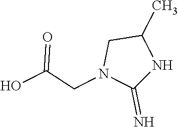 |
| 2-(2-imino-5-methyl- imidazolidin-1-yl)acetic acid | 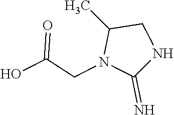 |
| 2-(2-imino-5-oxoimid- azolidin-1-yl)acetic acid | 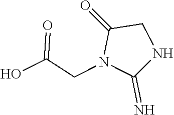 [SMILES: N=C1NCC(=O)N1CC(=O)O] |
| 2-(2-iminopyrrolidin- 3-yl)acetic acid | 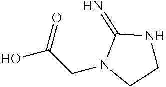 |
| 2-{2-[(prop-2-en-1- yl)amino]-4,5-dihydro- 1H-imidazol-1-yl}- acetic acid | 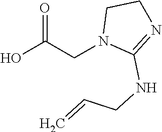 |
| 2-[2-(methylamino)- 4,5-dihydro-1H- imidazol-1-yl]acetic acid | 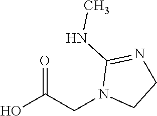 |
| 2-[2-iminopyrrolidin- 3-ylidene]acetic acid | 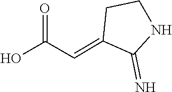 |
| 2-imino-1,3-diaza- bicyclo[3.2.0]heptane- 7-carboxylic acid | 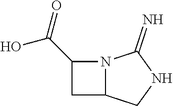 [SMILES: N=C1NCC2CC(C(=O)O)N12] |
| 3-carbamimidoyl-3- methylprop-2-enoic acid | 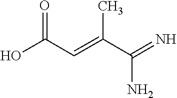 |
| N-(benzenesulfonyl)- 2-(1-methylguanidino)- acetamide | 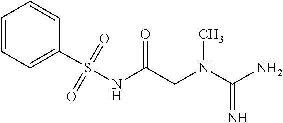 [SMILES: CN(CC(=O)NS(=O)(=O)c1ccccc1)C(=N)N] |
| [carbamimidoyl(meth- yl)carbamoyl]formic acid | 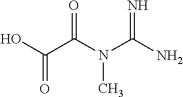 |
| 2-(2-amino-5-methyl- 6-oxo-1,6-dihydro- pyrimidin-1-yl)acetic acid | 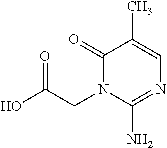 |
| 2-(3-imino-1,2,4- oxadiazolidin-4-yl)- acetic acid | 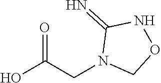 |
| 2-[{{[(3-chlorophen- yl)carbamoyl]amino}- methanimidoyl)(meth- yl)amino]acetic acid | 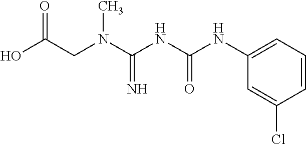 [SMILES: CN(CC(=O)O)C(=N)NC(=O)Nc1cccc(Cl)c1] |
| 2-[1-(bromomethyl)- guanidino]acetic acid | 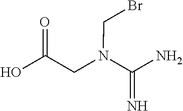 |
| 2-[1-methyl-2-(phos- phonooxy)guanidino]- acetic acid | 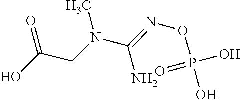 |
| 2-[3-(carboxymethyl)- 2-iminoimidazolidin- 1-yl]acetic acid | 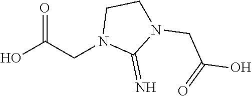 [SMILES: N=C1N(CC(=O)O)CCN1CC(=O)O] |
| 2-{[amino(sulfoimino)- methyl](methyl)amino}- acetic acid | 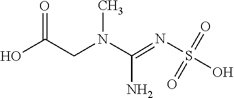 |
| 3-(1-methylguanidino)- prop-2-enoic acid | 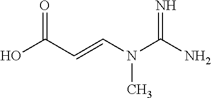 |
| 3-(carbamimidoyl- sulfanyl)-2-methyl- propanoic acid | 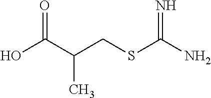 |
| 3-(carbamimidoyl- sulfanyl)butanoic acid | 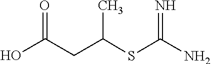 |
| 4-carbamimid- amidobut-2-enoic acid | 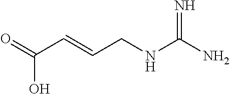 |
| 1,3-diamino-2-imino- imidazolidin-4-one | 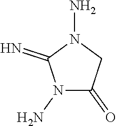 |
| 3-amino-2-hydrazinyl- ideneimidazolidin-4- one | 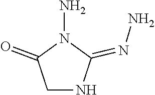 |
| 3-hydrazinylidene- 1,2,4-triazinan-6- one | 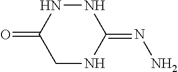 |
| 2-(3-aminoguanidino)- acetic acid | 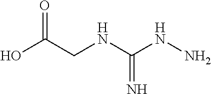 |
| 2-(3-nitroguanidino)- acetic acid | 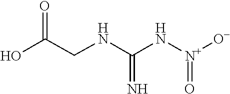 [SMILES: N=C(NCC(=O)O)N[N+](=O)[O-]] |
| 2-(carbamimidamido- amino)propanoic acid | 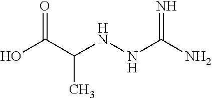 |
| 2-[3-(methylidene- amino)guanidino]acetic acid | 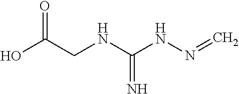 |
| 2-[1,2-diaminoguan- idino]acetic acid | 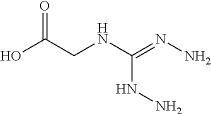 |
| benzyl 2-(carbamimid- amidoamino)acetate | 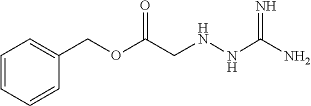 [SMILES: N=C(N)NNCC(=O)OCc1ccccc1] |
| 2-oxoazetidine-1- carboximidamide | 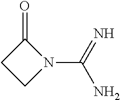 |
| (2R,3R,4R,5R)-4-[(3- carbamimidamido- propanoyl)oxy]-5-(5- fluoro-2-oxo-4- {[(pentyloxy)carbon- yl]amino}-1,2-dihydro- pyrimidin-1-yl)-2- methyloxolan-3-yl-3- carbamimidamido- propanoate | 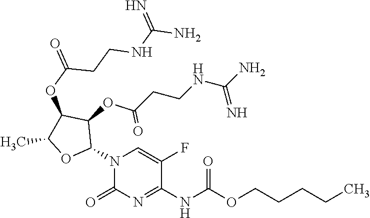 [SMILES: CCCCCOC(=O)Nc1nc(=O)n([C@@H]2O[C@H](C)[C@@H](OC(=O)CCNC(=N)N)[C@H]2OC(=O)CCNC(=N)N)cc1F] |
| 1-[(3-carbamimid- amidopropanoyl)oxy]- 3-[{{1-[(2R,3R,4S,5R)- 3,4-dihydroxy-5-meth- yloxolan-2-yl]-5-fluoro- 2-oxo-1,2-dihydro- pyrimidin-4-yl}- carbamoyl)oxy]propan- 2-yl-3-carbamimid- amidopropanoate | 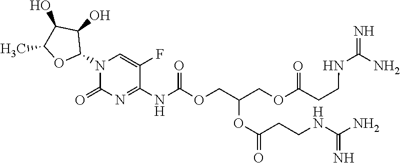 [SMILES: C[C@H]1O[C@@H](n2cc(F)c(NC(=O)OCC(COC(=O)CCNC(=N)N)OC(=O)CCNC(=N)N)nc2=O)[C@H](O)[C@@H]1O] |
| 1-(pyridin-2-yl)- azetidin-2-one | 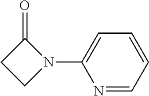 [SMILES: O=C1CCN1c1ccccn1] |
| 1-(pyridin-2-yl)- azetidine-3-carbox- ylic acid | 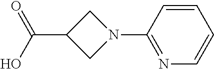 [SMILES: O=C(O)C1CN(c2ccccn2)C1] |
| 2-[1-(pyridin-2-yl)- azetidin-3-yl]acetic acid | 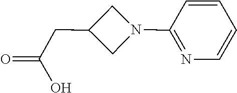 [SMILES: O=C(O)CC1CN(c2ccccn2)C1] |
| (2S)-2-amino-3- {[{amino[(2-carboxy- ethyl)aminomethyl- idene}amino]sulfanyl}- propanoic acid | 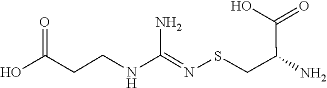 [SMILES: N/C(=N\SC[C@@H](N)C(=O)O)NCCC(=O)O] |
| (2S)-3-{[{amino[(2- carboxyethyl)amino]- methylidene}amino]- sulfanyl}-2-acetamido- propanoic acid | 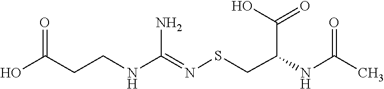 [SMILES: CC(=O)N[C@H](CS/N=C(\N)NCCC(=O)O)C(=O)O] |
| 3-[2-[(carboxymethyl)- sulfanyl]carbamimid- amido]propanoic acid | 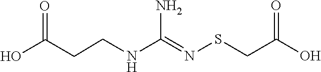 [SMILES: N/C(=N\SCC(=O)O)NCCC(=O)O] |
| 3-[2-[(2-aminoethyl)- sulfanyl]carbamimid- amido]propanoic acid | 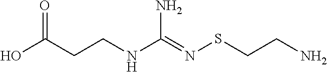 [SMILES: NCCS/N=C(\N)NCCC(=O)O] |
| (2R)-2-amino-4- {[{amino[(2-carboxy- ethyl)amino]methyl- idene}amino]sulfanyl}- butanoic acid | 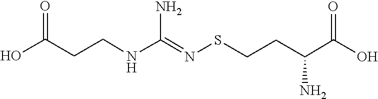 [SMILES: N/C(=N\SCC[C@@H](N)C(=O)O)NCCC(=O)O] |
| 3-[2-[(2-sulfoethyl)- sulfanyl]carbamimid- amido]propanoic acid | 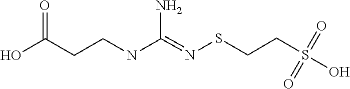 |
| 3-[2-{[(2S)-2-amino- 2-carboxyethyl]- sulfanyl}carbamimid- amido]butanoic acid | 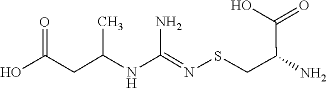 [SMILES: CC(CC(=O)O)N/C(N)=N/SC[C@@H](N)C(=O)O] |
| 3-[2-{[(2S)-2-carboxy- 2-acetamidoethyl]- sulfanyl}carbamimid- amido]butanoic acid | 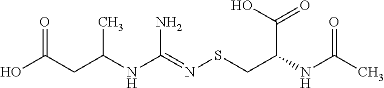 [SMILES: CC(=O)N[C@H](CS/N=C(\N)NC(C)CC(=O)O)C(=O)O] |
| 3-[2-[(carboxymethyl)- sulfanyl]carbamimid- amido]butanoic acid | 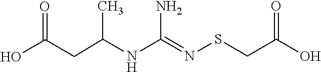 |
| 3-[2-[(2-aminoethyl)- sulfanyl]carbamimid- amido]butanoic acid | 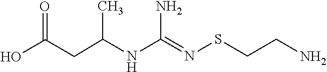 |
| (2R)-2-amino-4- {[{amino[(1-carboxy- propan-2-yl)amino]- methylidene}amino]- sulfanyl}butanoic acid | 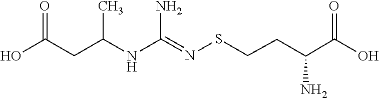 [SMILES: CC(CC(=O)O)N/C(N)=N/SCC[C@@H](N)C(=O)O] |
| 3-[2-[(2-sulfoethyl)- sulfanyl]carbamimid- amido]butanoic acid | 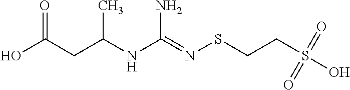 [SMILES: CC(CC(=O)O)N/C(N)=N/SCCS(=O)(=O)O] |
| (2S)-2-amino-3-({[1- (carboxymethyl)imid- azolidin-2-ylidene]- amino}sulfanyl)- propanoic acid | 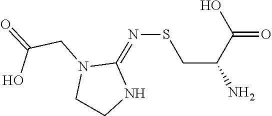 [SMILES: N[C@H](CS/N=C1\NCCN1CC(=O)O)C(=O)O] |
| (2S)-3-({[1-(carboxy- methyl)imidazolidin- 2-ylidene]amino}- sulfanyl)-2-acetamido- propanoic acid | 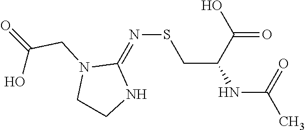 [SMILES: CC(=O)N[C@H](CS/N=C1\NCCN1CC(=O)O)C(=O)O] |
| 2-({[1-(carboxymeth- yl)imidazolidin-2- ylidene]amino}sulfan- yl)acetic acid | 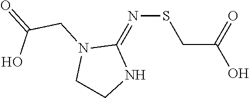 [SMILES: O=C(O)CS/N=C1\NCCN1CC(=O)O] |
| 2-[2-{[(2-aminoethyl)- sulfanyl]imino}imid- azolidin-1-yl]acetic acid | 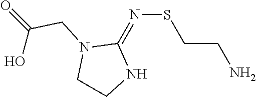 [SMILES: NCCS/N=C1\NCCN1CC(=O)O] |
| (2R)-2-amino-4-({[1- (carboxymethyl)imid- azolidin-2-ylidene]- amino}sulfanyl)- butanoic acid | 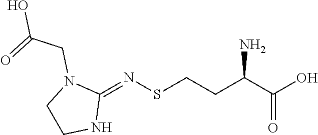 [SMILES: N[C@H](CCS/N=C1\NCCN1CC(=O)O)C(=O)O] |
| 2-[2-{[(2-sulfoethyl)- sulfanyl]imino}imid- azolidin-1-yl]acetic acid | 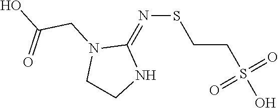 [SMILES: O=C(O)CN1CCN/C1=N\SCCS(=O)(=O)O] |
| (2S)-2-amino-3-({[1- (carboxymethyl)-1,3- diazinan-2-ylidene]- amino}sulfanyl)- propanoic acid | 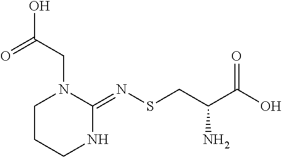 [SMILES: N[C@H](CS/N=C1\NCCCN1CC(=O)O)C(=O)O] |
| (2S)-3-({[1-(carboxy- methyl)-1,3-diazinan- 2-ylidene]amino}- sulfanyl)-2-acetamido- propanoic acid | 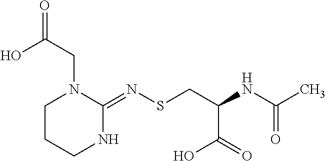 [SMILES: CC(=O)N[C@H](CS/N=C1\NCCCN1CC(=O)O)C(=O)O] |
| 2-({[1-(carboxymeth- yl)-1,3-diazinan-2- ylidene]amino}sulfan- yl)acetic acid | 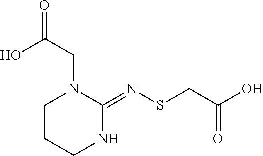 [SMILES: O=C(O)CS/N=C1\NCCCN1CC(=O)O] |
| 2-[2-{[(2-aminoethyl)- sulfanyl]imino}-1,3- diazinan-1-yl]acetic acid | 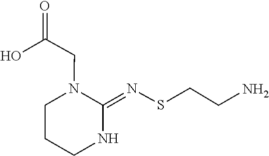 [SMILES: NCCS/N=C1\NCCCN1CC(=O)O] |
| (2R)-2-amino-4-({[1- (carboxymethyl)-1,3- diazinan-2-ylidene]- amino}sulfanyl)- butanoic acid | 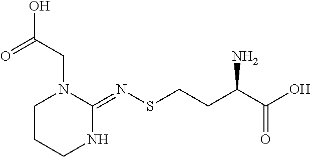 [SMILES: N[C@H](CCS/N=C1\NCCCN1CC(=O)O)C(=O)O] |
| 2-[2-{[(2-sulfoethyl)- sulfanyl]imino}-1,3- diazinan-1-yl]acetic acid | 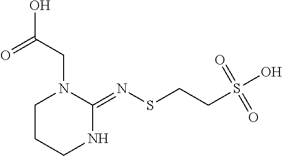 [SMILES: O=C(O)CN1CCCN/C1=N\SCCS(=O)(=O)O] |
| (2S)-2-amino-3- {[{amino[(carboxy- methyl)(methyl)- amino]methylidene}- amino]sulfanyl}- propanoic acid | 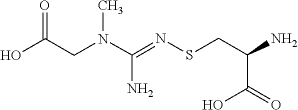 [SMILES: CN(CC(=O)O)/C(N)=N/SC[C@@H](N)C(=O)O] |
| (2S)-3{[{amino- [(carboxymethyl)- (methyl)amino]- methylidene}- amino]sulfanyl}- 2-acetamido- propanoic acid | 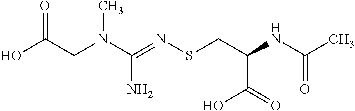 [SMILES: CC(=O)N[C@H](CS/N=C(\N)N(C)CC(=O)O)C(=O)O] |
| 2-{[{[(carboxy- methyl)(methyl)- amino](amino)- methylidene}- amino]sulfanyl}- acetic acid | 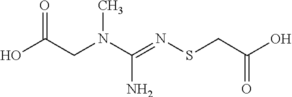 |
| 2-[2-[(2-aminoethyl)- sulfanyl]-1-methyl- guanidino]acetic acid | 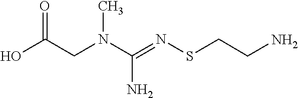 |
| (2R)-2-amino-4- {[{amino[(carboxy- methyl)(methyl)- amino]methylidene}- amino]sulfanyl}- butanoic acid | 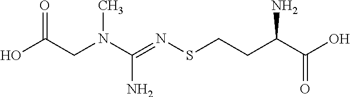 [SMILES: CN(CC(=O)O)/C(N)=N/SCC[C@@H](N)C(=O)O] |
| 2-[1-methyl-2-[(2- sulfoethyl)sulfanyl]- guanidino]acetic acid | 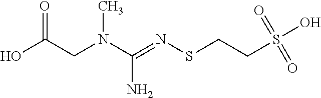 [SMILES: CN(CC(=O)O)/C(N)=N/SCCS(=O)(=O)O] |
| (2S)-2-amino-3- {[{[(carboxymeth- yl)(methyl)amino]- (methylamino)- methylidene}- amino]sulfanyl}- propanoic acid | 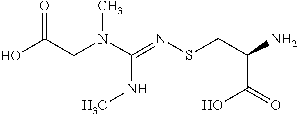 [SMILES: CN/C(=N\SC[C@@H](N)C(=O)O)N(C)CC(=O)O] |
| (2S)-3-{[{[(carboxy- methyl)(methyl)- amino](methylamino)- methylidene}amino]- sulfanyl}-2-acetamido- propanoic acid | 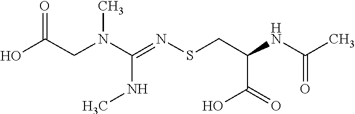 [SMILES: CN/C(=N\SC[C@@H](NC(C)=O)C(=O)O)N(C)CC(=O)O] |
| 2-{[{[(carboxy- methyl)(methyl)- amino](methylamino)- methylidene}amino]- sulfanyl}acetic acid | 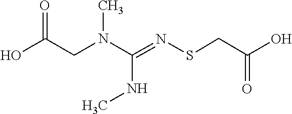 |
| 2-[2-[(2-aminoethyl)- sulfanyl]-1,3-dimethyl- guanidino]acetic acid | 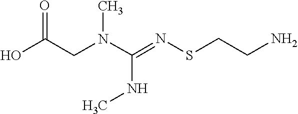 |
| (2R)-2-amino-4- {[{[(carboxymethyl)- (methyl)amino]- (methylamino)- methylidene}- amino]sulfanyl}- butanoic acid | 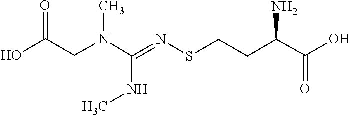 [SMILES: CN/C(=N\SCC[C@@H](N)C(=O)O)N(C)CC(=O)O] |
| 2-[1,3-dimethyl-2- [(2-sulfoethyl)- sulfanyl]guanidino]- acetic acid | 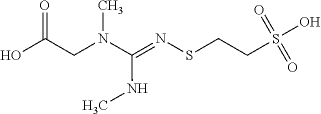 [SMILES: CN/C(=N\SCCS(=O)(=O)O)N(C)CC(=O)O] |
| 5-amino-4-(amino- methyl)pentanoic acid | 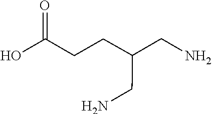 |
| 3-(1H-pyrazol-1-yl)- propanoic acid | 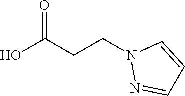 |
| 3-{4-amino-1H- pyrazolo[3,4-d]- pyrimidin-1-yl}- propanoic acid | 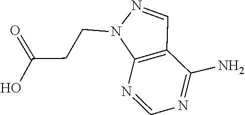 [SMILES: Nc1ncnc2c1cnn2CCC(=O)O] |
| 3-{4-amino-2H- pyrazolo[3,4-d]- pyrimidin-2-yl}- propanoic acid | 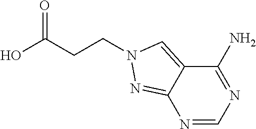 [SMILES: Nc1ncnc2nn(CCC(=O)O)cc12] |
| 3-amino-1-(carbox- ylatomethyl)pyridin- 1-ium | 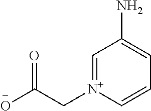 |
| 3-(carboxylatometh- yl)-1-(carboxymeth- yl)-2-methyl-4,5- dihydro-1H-imidazol- 3-ium | 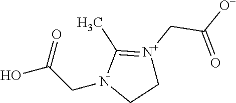 |

Additional non-limiting examples of agents that can be used in the methods and compositions provided herein are described, e.g., in U.S. Pat. Nos. 10,308,597 B2, 10,717,704 B2 the teachings of each of which are incorporated herein by reference in their entirety. β-Guanidinopropionic acid

In some embodiments of any of the aspects, the creatine transporter inhibitor is (β-Guanidinopropionic acid (also referred to herein as β-GPA or “RGX-202”), or a pharmaceutically acceptable salt thereof (e.g., RGX-202-01). β-GPA is a creatine analog identified by CAS number 353-09-3. β-GPA (or RGX-202) provided herein is a small molecule having the structure:

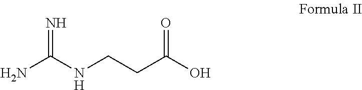

β-GPA (Formula II) is zwitterionic and highly soluble in water (greater than about 50 mg/mL) but has low solubility in organic solvents.

Pharmaceutically acceptable salt forms can include but are not limited to those described, e.g., in U.S. Pat. Nos. 9,884,813, 9,827,217, and 10,512,623, the teachings of which are incorporated herein by reference in its entirety.

### RGX-202-01

In some embodiments of any of the aspects, the creatine transporter inhibitor is RGX-202-01. RGX-202-01 is a small molecule provided herein that is a pharmaceutically optimized form of RGX-202 (also known as the endogenous compound, β-guanidinopropionic acid or β-GPA) suitable for clinical administration. RGX-202-01 is also a small molecule inhibitor of the creatine transporter, SLC6A8.

In some embodiments of any of the aspects, the one or more creatine transporter inhibitor for use in the methods and compositions provided herein is selected from the group consisting of: β-Guanidinopropionic acid, N-methylamidino-N-methylglycine, 1-carboxymethyl-2-imino-hexahydropyrimidine (cyclocreatine), DL-alpha-guanidinopropionic acid, N-methyl-N-amidino-beta-alanine, N-ethyl-N-amidinoglycine, DL-alpha-guanidinobutyric acid, DL-beta-guanidinobutyric acid, gamma-guanidinobutyric acid, guanidinoacetic acid, RGX-202-01, combinations and pharmaceutically acceptable salts thereof.

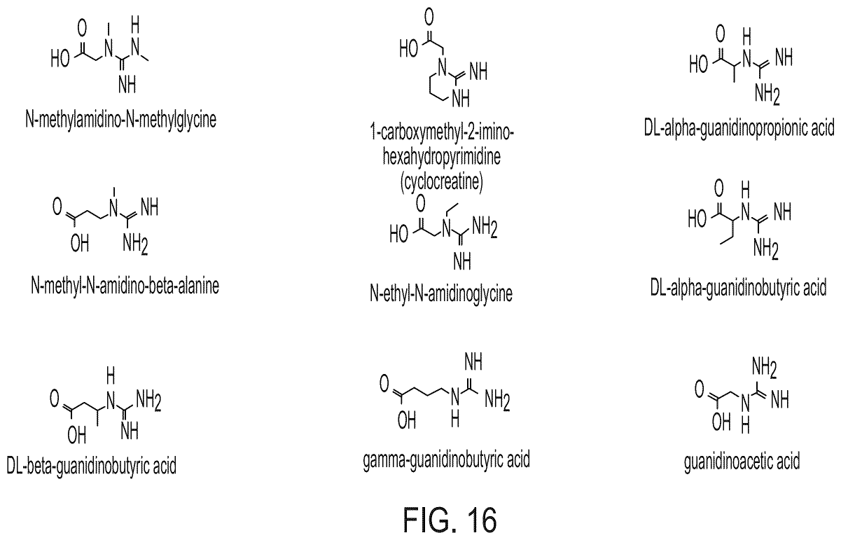

Additional examples of the chemical structures of the above creatine transporter inhibitors are found, e.g., in FIG. 16.

Any one of the creatine transporter inhibitors provided above or in Table 2 can be used in combination with any one or more of the additional agents or KRAS inhibitors provided herein to reduce cancer cell proliferation or tumor growth and/or as a treatment for cancer in a subject. Combination therapies including such agents are discussed further below.

### Combination Therapy

Therapeutic combinations featuring an SLC6A8 transporter inhibitor (e.g., β-Guanidinoproprionic acid, RGX-202, RGX-202-01) and a KRAS inhibitor (e.g., Sotorasib, Adagrasib, MRTX1133, RMC-6291, and RMC-6236) and/or another chemotherapeutic (e.g., FOLFIRI, FOLFOX, bevacizumab, 5-fluorouracil, leflunomide, irinotecan, and/or oxaliplatin) are provided by this disclosure. In embodiments, therapeutic combinations provided herein can be used in combination with one or more additional anti-cancer therapy (e.g. surgery, radiation therapy), and/or one or more additional therapeutic agents.

Specifically, combination therapy encompasses both co-administration (e.g., administration of a co-formulation or simultaneous administration of separate therapeutic compositions) and serial or sequential administration, provided that administration of one therapeutic agent is conditioned in some way on administration of another therapeutic agent. For example, one therapeutic agent (e.g., β-GPA) may be administered only after a different therapeutic agent (e.g., a KRAS inhibitor of Table 1 and/or an agent provided in Table 3 below) has been administered and allowed to act for a prescribed period of time.

Non-limiting examples of agents that can be combined with a creatine transporter inhibitor provided herein are provided in Table 3 below.

TABLE 3
Exemplary Anti-Cancer and Antiproliferative Agents

| Drug Class | Agents | |
|---|---|---|
| Alkylating agents | Busulfan | Chlorambucil |
| | dacarbazine | procarbazine |
| | ifosfamide | altretamine |
| | hexamethylmelamine | estramustine phosphate |
| | thiotepa | mechlorethamine |
| | dacarbazine | streptozocin |
| | lomustine | temozolomide |
| | cyclophosphamide | Semustine |
| Platinum agents | spiroplatin | lobaplatin (Aeterna) |
| | tetraplatin | satraplatin (Johnson |
| | ormaplatin | Matthey) |
| | iproplatin | BBR-3464 (Hoffmann-La |
| | ZD-0473 (AnorMED) | Roche) |
| | oxaliplatin | SM-11355 (Sumitomo) |
| | carboplatin | AP-5280 (Access) |
| | | cisplatin |
| Antimetabolites | azacytidine | trimetrexate |
| | Floxuridine | deoxycoformycin |
| | 2-chlorodeoxyadenosine | pentostatin |
| | 6-mercaptopurine | hydroxyurea |
| | 6-thioguanine | decitabine (SuperGen) |
| | cytarabine | clofarabine (Bioenvision) |
| | 2-fluorodeoxy cytidine | irofulven (MGI Pharma) |
| | methotrexate | DMDC (Hoffmann-La |
| | tomudex | Roche) |
| | fludarabine | ethynylcytidine (Taiho) |
| | raltitrexed | gemcitabine |
| | | capecitabine |
| Topoisomerase inhibitors | amsacrine | exatecan mesylate (Daiichi) |
| | epirubicin | quinamed (ChemGenex) |
| | etoposide | gimatecan (Sigma-Tau) |
| | teniposide or mitoxantrone | diflomotecan (Beaufour- |
| | 7-ethyl-10-hydroxy-camptothecin | Ipsen) |
| | dexrazoxanet (TopoTarget) | TAS-103 (Taiho) |
| | pixantrone (Novuspharma) | elsamitrucin (Spectrum) |
| | rebeccamycin analogue (Exelixis) | J-107088 (Merck & Co) |
| | BBR-3576 (Novuspharma) | BNP-1350 (BioNumerik) |
| | rubitecan (SuperGen) | CKD-602 (Chong Kun |
| | irinotecan (CPT-11) | Dang) |
| | topotecan | KW-2170 (Kyowa Hakko) |
| | | hydroxycamptothecin (SN- |
| | | 38) |
| Antitumor antibiotics | valrubicin | azonafide |
| | therarubicin | anthrapyrazole |
| | idarubicin | oxantrazole |
| | rubidazone | losoxantrone |
| | plicamycin | MEN-10755 (Menarini) |
| | porfiromycin | GPX-100 (Gem |
| | mitoxantrone (novantrone) | Pharmaceuticals) |
| | amonafide | Epirubicin |
| | | mitoxantrone |
| | | doxorubicin |
| Antimitotic | colchicine | E7010 (Abbott) |
| agents | vinblastine | PG-TXL (Cell |
| | vindesine | Therapeutics) |
| | dolastatin 10 (NCI) | IDN 5109 (Bayer) |
| | rhizoxin (Fujisawa) | A 105972 (Abbott) |
| | mivobulin (Warner-Lambert) | A 204197 (Abbott) |
| | cemadotin (BASF) | LU 223651 (BASF) |
| | RPR 109881A (Aventis) | D 24851 (ASTAMedica) |
| | TXD 258 (Aventis) | ER-86526 (Eisai) |
| | epothilone B (Novartis) | combretastatin A4 (BMS) |
| | T 900607 (Tularik) | isohomohalichondrin-B |
| | T 138067 (Tularik) | (PharmaMar) |
| | cryptophycin 52 (Eli Lilly) | ZD 6126 (AstraZeneca) |
| | vinflunine (Fabre) | AZ10992 (Asahi) |
| | auristatin PE (Teikoku Hormone) | IDN-5109 (Indena) |
| | BMS 247550 (BMS) | AVLB (Prescient |
| | BMS 184476 (BMS) | NeuroPharma) |
| | BMS 188797 (BMS) | azaepothilone B (BMS) |
| | taxoprexin (Protarga) | BNP-7787 (BioNumerik) |
| | SB 408075 (GlaxoSmithKline) | CA-4 prodrug (OXiGENE) |
| | Vinorelbine | dolastatin-10 (NIH) |
| | Trichostatin A | CA-4 (OXiGENE) |
| | | docetaxel |
| | | vincristine |
| | | paclitaxel |
| Aromatase inhibitors | aminoglutethimide | YM-511 (Yamanouchi) |
| | atamestane (BioMedicines) | formestane |
| | letrozole | exemestane |
| | anastrazole | |
| Thymidylate synthase inhibitors | pemetrexed (Eli Lilly) | nolatrexed (Eximias) |
| | ZD-9331 (BTG) | CoFactor ™ (BioKeys) |
| DNA antagonists | trabectedin (PharmaMar) | edotreotide (Novartis) |
| | glufosfamide (Baxter International) | mafosfamide (Baxter |
| | albumin + 32P (Isotope Solutions) | International) |
| | thymectacin (NewBiotics) | apaziquone (Spectrum |
| | | Pharmaceuticals) |
| | | O6 benzyl guanine |
| | | (Paligent) |
| Farnesyltransferase inhibitors | arglabin (NuOncology Labs) | tipifarnib (Johnson & |
| | lonafarnib (Schering-Plough) | Johnson) |
| | BAY-43-9006 (Bayer) | perillyl alcohol (DOR |
| | | BioPharma) |
| Pump inhibitors | CBT-1 (CBA Pharma) | zosuquidar trihydrochloride |
| | tariquidar (Xenova) | (Eli Lilly) |
| | MS-209 (Schering AG) | biricodar dicitrate (Vertex) |
| Histone acetyltransferase | tacedinaline (Pfizer) | pivaloyloxymethyl butyrate |
| inhibitors | SAHA (Aton Pharma) | (Titan) |
| | MS-275 (Schering AG) | depsipeptide (Fujisawa) |
| Metalloproteinase inhibitors | Neovastat (Aeterna Laboratories) | CMT-3 (CollaGenex) |
| | marimastat (British Biotech) | BMS-275291 (Celltech) |
| Ribonucleoside reductase | gallium maltolate (Titan) | tezacitabine (Aventis) |
| inhibitors | triapine (Vion) | didox (Molecules for |
| | | Health) |
| TNF alpha agonists/antagonists | virulizin (Lorus Therapeutics) | revimid (Celgene) |
| | CDC-394 (Celgene) | |
| Endothelin A receptor antagonist | atrasentan (Abbott) | YM-598 (Yamanouchi) |
| | ZD-4054 (AstraZeneca) | |
| Retinoic acid receptor agonists | fenretinide (Johnson & Johnson) | alitretinoin (Ligand) |
| | LGD-1550 (Ligand) | |
| Immuno-modulators | interferon | dexosome therapy (Anosys) |
| | oncophage (Antigenics) | pentrix (Australian Cancer |
| | GMK (Progenics) | Technology) |
| | adenocarcinoma vaccine (Biomira) | ISF-154 (Tragen) |
| | CTP-37 (AVI BioPharma) | cancer vaccine (Intercell) |
| | IRX-2 (Immuno-Rx) | norelin (Biostar) |
| | PEP-005 (Peplin Biotech) | BLP-25 (Biomira) |
| | synchrovax vaccines (CTL Immuno) | MGV (Progenics) |
| | melanoma vaccine (CTL Immuno) | β-alethine (Dovetail) |
| | p21 RAS vaccine (GemVax) | CLL therapy (Vasogen) |
| | MAGE-A3 (GSK) | Ipilimumab (BMS), |
| | nivolumab (BMS) | CM-10 (cCam |
| | abatacept (BMS) | Biotherapeutics) |
| | pembrolizumab | MPDL3280A (Genentech) |
| | atezolizumab | MEDI4736 |
| | durvalumab | adenosine inhibitors |
| Hormonal and antihormonal | estrogens | dexamethasone |
| agents | conjugated estrogens | prednisone |
| | ethinyl estradiol | methylprednisolone |
| | chlortrianisen | prednisolone |
| | idenestrol | aminoglutethimide |
| | hydroxyprogesterone caproate | leuprolide |
| | medroxyprogesterone | octreotide |
| | testosterone | mitotane |
| | testosterone propionate; | P-04 (Novogen) |
| | fluoxymesterone | 2-methoxyestradiol |
| | methyltestosterone | (EntreMed) |
| | diethylstilbestrol | arzoxifene (Eli Lilly) |
| | megestrol | tamoxifen |
| | bicalutamide | toremofine |
| | flutamide | goserelin |
| | nilutamide | Leuporelin |
| | | bicalutamide |
| Photodynamic agents | talaporfin (Light Sciences) | Pd-bacteriopheophorbide |
| | Theralux (Theratechnologies) | (Yeda) |
| | motexafin gadolinium (Pharmacyclics) | lutetium texaphyrin |
| | | (Pharmacyclics) |
| | | hypericin |
| Kinase Inhibitors | imatinib (Novartis) | EKB-569 (Wyeth) |
| | leflunomide (Sugen/Pharmacia) | kahalide F (PharmaMar) |
| | ZD1839 (AstraZeneca) | CEP-701 (Cephalon) |
| | erlotinib (Oncogene Science) | CEP-751 (Cephalon) |
| | canertinib (Pfizer) | MLN518 (Millenium) |
| | squalamine (Genaera) | PKC412 (Novartis) |
| | SU5416 (Pharmacia) | Phenoxodiol (Novogen) |
| | SU6668 (Pharmacia) | C225 (ImClone) |
| | ZD4190 (AstraZeneca) | rhu-Mab (Genentech) |
| | ZD6474 (AstraZeneca) | MDX-H210 (Medarex) |
| | vatalanib (Novartis) | 2C4 (Genentech) |
| | PKI166 (Novartis) | MDX-447 (Medarex) |
| | GW2016 (GlaxoSmithKline) | ABX-EGF (Abgenix) |
| | EKB-509 (Wyeth) | IMC-1C11 (ImClone) |
| | trastuzumab (Genentech) | Tyrphostins |
| | OSI-774 (Tarceva ™) | Gefitinib (Iressa) |
| | CI-1033 (Pfizer) | PTK787 (Novartis) |
| | SU11248 (Pharmacia) | EMD 72000 (Merck) |
| | RH3 (York Medical) | Emodin |
| | Genistein | Radicinol |
| | Radicinol | Vemurafenib (B-Raf |
| | Met-MAb (Roche) | enzyme inhibitor, Daiichi |
| | PLK (polo like kinase) inhibitors | Sankyo) |
| Additional antiproliferative | bevacizumab | Tyrphostin AG |
| Agents | angiogenesis inhibitors | PD-1 inhibitors |
| | rituximab (CD20 antibody, Genentech | CTLA-4 inhibitors |
| | dabrafenib | sorafenib |
| | midostaurin (PKC inhibitor, Novartis) | CRS-207 |
| | bryostatin-1 (PKC stimulant, GPC | CHS-828 (cytotoxic agent, |
| | Biotech) | Leo) |
| | CDA-II (apoptosis promotor, Everlife) | trans-retinoic acid |
| | SDX-101 (apoptosis promotor, | (differentiator, NIH) |
| | Salmedix) | MX6 (apoptosis promotor, |
| | | MAXIA) |
| | | apomine (apoptosis |
| | | promotor, ILEX Oncology) |
| dihydroorotate dehydrogenase | atovaquone, brequinar sodium, | |
| (DHODH) inhibitor | leflunomide, teriflunomide, BAY- | |
| | 2402234, AG-636, | |
| tyrosine phosphatase 2 (SHP) | JAB-3068, JAB-3312 (Jacobio) | |
| inhibitors | TNO-155 (Novartis) | |
| | RLY-1971 (Relay Therapeutics) | |
| KRAS mutant inhibitors | Sotorasib (LUMAKRAS ™, AMG- | |
| | 510) | |
| | Adagrasib (MRTX849) | |
| | MRTX1133 | |
| | RMC-6291 | |
| | RMC-6236 | |

In some embodiments of any of the aspects, the one or more additional agent is a KRAS inhibitor (e.g., those listed in Table 1), a SHP2 inhibitor, a HER2 inhibitor, an EGFR inhibitor, a SOS1 inhibitor, a Raf inhibitor, a MEK inhibitor, an ERK inhibitor, a P13K inhibitor, a Polo-like kinase 1 (PLKI) inhibitor, an adenosine inhibitor, a PTEN inhibitor, an AKT inhibitor, an mTORCI inhibitor, a BRAF inhibitor, a PD-L1 inhibitor, a PD-1 inhibitor, a CDK4/6 inhibitor, a dihydroorotate dehydrogenase (DHODH) inhibitor, or any combination thereof.

Exemplary EGFR inhibitors include, but are not limited to, Erlotinib (Tarceva), Afatinib (Gilotrif), Gefitinib (Iressa), Osimertinib (Tagrisso), Dacomitinib (Vizimpro), and Panitumumab (Vectibix). Non-limiting examples of EGFR inhibitors that can be used in the methods provided herein include those described below (i.e., Erlotinib, Osimertinib, Neratinib, Gefitinib, Cetuximab, Panitumurnab, Dacomitinib, Lapatinib, Necitumurnab, Mobocertinib, and Vandetanib), pharmaceutical salts, analogs, derivatives, and combinations thereof.

Erlotinib (Tarceva)—Erlotinib is a tyrosine kinase receptor inhibitor that is used in the therapy of advanced or metastatic pancreatic or non-small cell lung cancer. Erlotinib is a quinazoline derivative with antineoplastic properties. Competing with adenosine triphosphate, erlotinib reversibly binds to the intracellular catalytic domain of epidermal growth factor receptor (EGFR) tyrosine kinase, thereby reversibly inhibiting EGFR phosphorylation and blocking the signal transduction events and tumorigenic effects associated with EGFR activation.

Osimertinib (Tagrisso)—Tagrisso (osimertinib) is an EGFR-TKI, a targeted cancer therapy, designed to inhibit both the activating, sensitizing mutations (EGFRm), and T790M, a genetic mutation responsible to EGFR-TKI treatment resistance. Tagrisso (osimertinib) is kinase inhibitor of the epidermal growth factor receptor (EGFR), which binds irreversibly to certain mutant forms of EGFR (T790M, L858R, and exon 19 deletion) at approximately 9-fold lower concentrations than wild-type.

Neratinib (Nerlynx)—Neratinib is a potent, irreversible tyrosine kinase inhibitor (TKI) of HER1, HER2, and HER4. Neratinib irreversibly binds to the intercellular signaling domain of HER1, HER2, HER3, and epithelial growth factor receptor, and inhibits phosphorylation and several HER downstream signaling pathways. The result is decreased proliferation and increased cell death.

Gefitinib (Iressa)—Gefitinib is an inhibitor of the epidermal growth factor receptor (EGFR) tyrosine kinase that binds to the adenosine triphosphate (ATP)-binding site of the enzyme.

Cetuximab (Erbitux)—Erbitux is a recombinant, human/mouse chimeric monoclonal antibody. The antibody binds to epidermal growth factor receptor (EGFR, HER1, c-ErbB-1) on both normal and tumor cells, and competitively inhibits the binding of epidermal growth factor (EGF) and other ligands, such as transforming growth factor-alpha. Erbitux is composed of the Fv regions of a murine anti-EGFR antibody with human IgG1 heavy and kappa light chain constant regions.

Panitumumab (Vectibix)—Vectibix binds specifically to EGFR on both normal and tumor cells, and competitively inhibits the binding of ligands for EGFR. Nonclinical studies show that binding of panitumumab to the EGFR prevents ligand-induced receptor autophosphorylation and activation of receptor-associated kinases, resulting in inhibition of cell growth, induction of apoptosis, decreased pro-inflammatory cytokine and vascular growth factor production, and internalization of the EGFR.

Dacomitinib (Vizimpro)—Vizimpro (dacomitinib) is an irreversible inhibitor of the kinase activity of the human EGFR family (EGFR/HER1, HER2, and HER4) and certain EGFR activating mutations (exon 19 deletion or the exon 21 L858R substitution mutation). In vitro dacomitinib also inhibits the activity of DDR1, EPHA6, LCK, DDR2, and MNK1 at clinically relevant concentrations.

Lapatinib (Tykerb)—Tykerb is an inhibitor of the intracellular tyrosine kinase domains of both Epidermal Growth Factor Receptor (EGFR [ErbB1]) and of Human Epidermal Receptor Type 2 (HER-2 [ErbB2]) receptors. When the binding site is blocked signal molecules can no longer attach there and activate the tyrosine kinase, an enzyme which functions to stimulate cell division.

Necitumumab (Portrazza)—Portrazza (necitumumab) is a recombinant human IgG1 monoclonal antibody that binds to the human epidermal growth factor receptor (EGFR) and blocks the binding of EGFR to its ligands. Expression and activation of EGFR has been correlated with malignant progression, induction of angiogenesis and inhibition of apoptosis.

Binding of necitumumab induces EGFR internalization and degradation in vitro. In vitro, binding of necitumumab also led to antibody-dependent cellular cytotoxicity (ADCC) in EGFR-expressing cells.

Mobocertinib (Exkivity)—Exkivity (mobocertinib) is a kinase inhibitor specifically designed to selectively target epidermal growth factor receptor (EGFR) Exon20 insertion mutations at lower concentrations than wild type (WT) EGFR. Two pharmacologically-active metabolites (AP32960 and AP32914) with similar inhibitory profiles to mobocertinib have been identified in the plasma after oral administration of mobocertinib. In vitro, mobocertinib also inhibited the activity of other EGFR family members (HER2 and HER4) and one additional kinase (BLK) at clinically relevant concentrations (IC50 values <2 nM).

Vandetanib (Caprelsa)—Vandetanib is a kinase inhibitor. Vandetanib inhibits the activity of tyrosine kinases including members of the epidermal growth factor receptor (EGFR) family, vascular endothelial cell growth factor (VEGF) receptors, rearranged during transfection (RET), protein tyrosine kinase 6 (BRK), TIE2, members of the EPH receptors kinase family, and members of the Src family of tyrosine kinases. Vandetanib inhibits endothelial cell migration, proliferation, survival and new blood vessel formation in in vitro models of angiogenesis.

Vandetanib inhibits EGFR-dependent cell survival in vitro. In addition, vandetanib inhibits epidermal growth factor (EGF)-stimulated receptor tyrosine kinase phosphorylation in tumor cells and endothelial cells and VEGF-stimulated tyrosine kinase phosphorylation in endothelial cells. In vivo vandetanib administration reduced tumor cell-induced angiogenesis, tumor vessel permeability, and inhibited tumor growth and metastasis in mouse models of cancer.

In some embodiments of any of the aspects, the one or more additional agent is selected from the group consisting of. Sotorasib (LUMAKRAS™, AMG-510), adagrasib (MRTX849), MRTX1133, RMC-6291, RMC-6236, atovaquone, brequinar sodium, leflunomide, teriflunomide, BAY-2402234, AG-636, leucovorin (folinic acid), 5-fluorouracil (5-FU), irinotecan, oxaliplatin, FOLFIRI, FOLFOX, combinations and pharmaceutically acceptable salts thereof.

In some embodiments of any of the aspects, the subject has been administered at least one prior anti-cancer therapy or agent prior to being administered the creatine transporter inhibitor and/or the one or more additional antiproliferative agent provided herein.

It will also be appreciated that the compounds and pharmaceutical compositions of the present invention can be formulated and employed in combination therapies, that is, the compounds and pharmaceutical compositions can be formulated with or administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (e.g., reducing cancer cell proliferation or tumor size in a subject), or they may achieve different effects (e.g., control of any adverse effects).

The term “therapeutic synergy” is used, for example, when the combination of two agents at given doses are more efficacious than the best of the two products alone, considering the same doses. In order to study therapeutic synergy, each combination can be compared to a single agent using estimates obtained from a two-way analysis of variance with repeated measurements (Time factor) on parameter tumor volume in vivo or cell proliferation.

### Pharmaceutical Compositions

Pharmaceutical compositions featuring an SLC6A8 transporter inhibitor (e.g., (β-Guanidinoproprionic acid, RGX-202) and a KRAS inhibitor (e.g., Sotorasib, Adagrasib, MRTX1133, RMC-6291, and RMC-6236) and/or another chemotherapeutic (e.g., FOLFIRI, FOLFOX, bevacizumab, 5-fluorouracil, leflunomide, irinotecan, and/or oxaliplatin) are formulated separately or in combination for delivery to a subject in need thereof. In some embodiments, a therapeutic combination is delivered together with a carrier that is pharmaceutically acceptable and is appropriate for delivering the compounds by the chosen route of administration. Suitable pharmaceutically acceptable carriers are those used conventionally with small molecules, such as diluents, excipients and the like. See, for example, “Remington s Pharmaceutical Sciences”, 17th Ed., Mack Publishing Company, Easton, Pa., 1995, for guidance on drug formulations.

Within the scope of this invention is a composition that contains a suitable carrier and one or more of the therapeutic agents described above. The composition can be a pharmaceutical composition that contains a pharmaceutically acceptable carrier, a dietary composition that contains a dietarily acceptable suitable carrier, or a cosmetic composition that contains a cosmetically acceptable carrier.

The term “pharmaceutical composition” refers to the combination of an active agent with a carrier, inert or active, making the composition especially suitable for diagnostic or therapeutic use in vivo or ex vivo. A “pharmaceutically acceptable carrier,” after administered to or upon a subject, does not cause undesirable physiological effects. The carrier in the pharmaceutical composition must be “acceptable” also in the sense that it is compatible with the active ingredient and can be capable of stabilizing it. One or more solubilizing agents can be utilized as pharmaceutical carriers for delivery of an active compound. Examples of a pharmaceutically acceptable carrier include, but are not limited to, biocompatible vehicles, adjuvants, additives, and diluents to achieve a composition usable as a dosage form. Examples of other carriers include colloidal silicon oxide, magnesium stearate, cellulose, sodium lauryl sulfate, and D&C Yellow #10.

As used herein, the term “pharmaceutically acceptable salt” refers to those salts which are, within the scope of sound medical judgment, suitable for use in contact with the tissues of humans and lower animals without undue toxicity, irritation, or allergic response, and are commensurate with a reasonable benefit/risk ratio. Pharmaceutically acceptable salts of amines, carboxylic acids, and other types of compounds, are well known in the art. For example, S. M. Berge, et al. describe pharmaceutically acceptable salts in detail in J Pharmaceutical Sciences, 66: 1-19 (1977), incorporated herein by reference. The salts can be prepared in situ during the final isolation and purification of the compounds of the invention, or separately by reacting a free base or free acid function with a suitable reagent, as described generally below. For example, a free base function can be reacted with a suitable acid. Furthermore, where the compounds of the invention carry an acidic moiety, suitable pharmaceutically acceptable salts thereof may, include metal salts such as alkali metal salts, e.g. sodium or potassium salts; and alkaline earth metal salts, e.g. calcium or magnesium salts. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts, include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, and valerate salts. Representative alkali or alkaline earth metal salts include sodium, lithium, potassium, calcium, and magnesium. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

As described above, the pharmaceutical compositions of the present invention additionally include a pharmaceutically acceptable carrier, which, as used herein, includes any and all solvents, diluents, or other liquid vehicle, dispersion or suspension aids, surface active agents, isotonic agents, thickening or emulsifying agents, preservatives, solid binders, and lubricants, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutical compositions and known techniques for the preparation thereof. Except insofar as any conventional carrier medium is incompatible with the compounds of the invention, such as by producing any undesirable biological effect or otherwise interacting in a deleterious manner with any other component(s) of the pharmaceutical composition, its use is contemplated to be within the scope of this invention. Some examples of materials which can serve as pharmaceutically acceptable carriers include, but are not limited to, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatine; talc; excipients such as cocoa butter and suppository waxes; oils such as peanut oil, cottonseed oil; safflower oil, sesame oil; olive oil; corn oil and soybean oil; glycols; such as propylene glycol; esters such as ethyl oleate and ethyl laurate; agar; natural and synthetic phospholipids, such as soybean and egg yolk phosphatides, lecithin, hydrogenated soy lecithin, dimyristoyl lecithin, dipalmitoyl lecithin, distearoyl lecithin, dioleoyl lecithin, hydroxylated lecithin, lysophosphatidylcholine, cardiolipin, sphingomyelin, phosphatidylcholine, phosphatidyl ethanolamine, diastearoyl phosphatidylethanolamine (DSPE) and its pegylated esters, such as DSPE-PEG750 and, DSPE-PEG2000, phosphatidic acid, phosphatidyl glycerol and phosphatidyl serine. Commercial grades of lecithin which are preferred include those which are available under the trade name Phosal® or Phospholipon® and include Phosal 53 MCT, Phosal 50 PG, Phosal 75 SA, Phospholipon 90H, Phospholipon 90G and Phospholipon 90 NG; soy-phosphatidylcholine (SoyPC) and DSPE-PEG2000 are particularly preferred; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

A pharmaceutical composition of this invention can be administered parenterally, orally, nasally, rectally, topically, or buccally. The term “parenteral” as used herein refers to subcutaneous, intracutaneous, intravenous, intramuscular, intraarticular, intraarterial, intrasynovial, intrasternal, intrathecal, intralesional, or intracranial injection, as well as any suitable infusion technique.

A sterile injectable composition can be a solution or suspension in a non-toxic parenterally acceptable diluent or solvent. Such solutions include, but are not limited to, 1,3-butanediol, mannitol, water, Ringer's solution, and isotonic sodium chloride solution. In addition, fixed oils are conventionally employed as a solvent or suspending medium (e.g., synthetic mono- or diglycerides). Fatty acid, such as, but not limited to, oleic acid and its glyceride derivatives, are useful in the preparation of injectables, as are natural pharmaceutically acceptable oils, such as, but not limited to, olive oil or castor oil, polyoxyethylated versions thereof. These oil solutions or suspensions also can contain a long chain alcohol diluent or dispersant such as, but not limited to, carboxymethyl cellulose, or similar dispersing agents. Other commonly used surfactants, such as, but not limited to, Tweens or Spans or other similar emulsifying agents or bioavailability enhancers, which are commonly used in the manufacture of pharmaceutically acceptable solid, liquid, or other dosage forms also can be used for the purpose of formulation.

In some embodiments of any of the aspects, one or more agents provided herein are formulated for oral administration. A composition for oral administration can be any orally acceptable dosage form including capsules, tablets, emulsions and aqueous suspensions, dispersions, and solutions. In the case of tablets, commonly used carriers include, but are not limited to, lactose and corn starch. Lubricating agents, such as, but not limited to, magnesium stearate, also are typically added. For oral administration in a capsule form, useful diluents include, but are not limited to, lactose and dried corn starch. When aqueous suspensions or emulsions are administered orally, the active ingredient can be suspended or dissolved in an oily phase combined with emulsifying or suspending agents. If desired, certain sweetening, flavoring, or coloring agents can be added.

For oral administration, the pharmaceutical compositions may take the form of, for example, tablets, lozenges, or capsules prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicles before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., ationd oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound.

Pharmaceutical compositions for topical administration according to the described invention can be formulated as solutions, ointments, creams, suspensions, lotions, powders, pastes, gels, sprays, aerosols, or oils. Alternatively, topical formulations can be in the form of patches or dressings impregnated with active ingredient(s), which can optionally include one or more excipients or diluents. In some preferred embodiments, the topical formulations include a material that would enhance absorption or penetration of the active agent(s) through the skin or other affected areas.

A topical composition contains a safe and effective amount of a dermatologically acceptable carrier suitable for application to the skin. A “cosmetically acceptable” or “dermatologically-acceptable” composition or component refers a composition or component that is suitable for use in contact with human skin without undue toxicity, incompatibility, instability, or allergic response. The carrier enables an active agent and optional component to be delivered to the skin at an appropriate concentration(s). The carrier thus can act as a diluent, dispersant, solvent, or the like to ensure that the active materials are applied to and distributed evenly over the selected target at an appropriate concentration. The carrier can be solid, semi-solid, or liquid. The carrier can be in the form of a lotion, a cream, or a gel, in particular one that has a sufficient thickness or yield point to prevent the active materials from sedimenting. The carrier can be inert or possess dermatological benefits. It also should be physically and chemically compatible with the active components described herein, and should not unduly impair stability, efficacy, or other use benefits associated with the composition.

Pharmaceutical compositions that may oxidize and lose biological activity, especially in a liquid or semisolid form, may be prepared in a nitrogen atmosphere or sealed in a type of capsule and/or foil package that excludes oxygen (e.g., Capsugel™) For administration by inhalation, the agents may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Capsules and cartridges of, e.g., gelatin, for use in an inhaler or insufflator may be formulated containing a powder mix of the agent and a suitable powder base such as lactose or starch.

Pharmaceutical compositions may be formulated for parenteral administration by injection, e.g., by bolus injection or continuous infusion. Formulations for injection may be presented in unit dosage form, e.g., in ampoules or in multi-dose containers, with an added preservative. The agents may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use. The agents may also be formulated in rectal compositions such as suppositories or retention enemas, e.g., containing conventional suppository bases such as cocoa butter or other glycerides.

In addition to the formulations described previously, pharmaceutical compositions may also be formulated as a depot preparation. Such long acting formulations may be administered by implantation (for example subcutaneously or intramuscularly) or by intramuscular injection.

Thus, for example, the agents may be formulated with suitable polymeric or hydrophobic materials (for example as an emulsion in an acceptable oil) or ion exchange resins, or as sparingly soluble derivatives, for example, as a sparingly soluble salt. Controlled release formula also includes patches, e.g., transdermal patches. Patches may be used with a sonic applicator that deploys ultrasound in a unique combination of waveforms to introduce drug molecules through the skin that normally could not be effectively delivered transdermally.

Pharmaceutical compositions can contain a non-dissolving, non-disintegrating slow-release suppository base consisting essentially of a linear polymer, such as methyl cellulose, polyvinyl pyrrolidone, and water.

Pharmaceutical compositions may be incorporated into gel formulations, which generally are semisolid systems consisting of either suspension made up of small inorganic particles (two-phase systems) or large organic molecules distributed substantially uniformly throughout a carrier liquid (single-phase gels). Single-phase gels can be made, for example, by combining the active agent, a carrier liquid and a suitable gelling agent such as tragacanth (at 2 to 5%), sodium alginate (at 2-10%), gelatin (at 2-15%), methylcellulose (at 3-5%), sodium carboxymethylcellulose (at 2-5%), carbomer (at 0.3-5%) or polyvinyl alcohol (at 10-20%) together and mixing until a characteristic semisolid product is produced. Other suitable gelling agents include methylhydroxycellulose, polyoxyethylene-polyoxypropylene, hydroxyethylcellulose, and gelatin. Although gels commonly employ aqueous carrier liquid, alcohols and oils can be used as the carrier liquid as well.

Pharmaceutical compositions may be incorporated into microemulsions, which generally are thermodynamically stable, isotropically clear dispersions of two immiscible liquids, such as oil and water, stabilized by an interfacial film of surfactant molecules (Encyclopedia of Pharmaceutical Technology (New York: Marcel Dekker, 1992), volume 9). For the preparation of microemulsions, surfactant (emulsifier), co-surfactant (co-emulsifier), an oil phase and a water phase are necessary. Suitable surfactants include any surfactants that are useful in the preparation of emulsions, e.g., emulsifiers that are typically used in the preparation of creams. The co-surfactant (or “co-emulsifier”) is generally selected from the group of polyglycerol derivatives, glycerol derivatives, and fatty alcohols. Preferred emulsifier/co-emulsifier combinations are generally although not necessarily selected from the group consisting of: glyceryl monostearate and polyoxyethylene stearate; polyethylene glycol and ethylene glycol palmitostearate; and caprylic and capric triglycerides and oleoyl macrogolglycerides. The water phase includes not only water but also, typically, buffers, glucose, propylene glycol, polyethylene glycols, preferably lower molecular weight polyethylene glycols (e.g., PEG 300 and PEG 400), and/or glycerol, and the like, while the oil phase will generally comprise, for example, fatty acid esters, modified vegetable oils, silicone oils, mixtures of mono- di- and triglycerides, mono- and di-esters of PEG (e.g., oleoyl macrogol glycerides), etc.

In some embodiments, a pharmaceutical formulation is provided for oral or parenteral administration, in which case the formulation may comprise an activating compound-containing microemulsion as described above, and may contain alternative pharmaceutically acceptable carriers, vehicles, additives, etc. particularly suited to oral or parenteral drug administration.

Alternatively, an activating compound-containing microemulsion may be administered orally or parenterally substantially as described above, without modification.

In some embodiments, the formulation comprising a compound/agent comprises one or more additional components, wherein the additional component is at least one of an osmolar component that provides an isotonic, or near isotonic solution compatible with human cells or blood, and a preservative.

In some embodiments, the osmolar component is a salt, such as sodium chloride, or a sugar or a combination of two or more of these components. In some embodiments, the sugar may be a monosaccharide such as dextrose, a disaccharide such as sucrose or lactose, a polysaccharide such as dextran 40, dextran 60, or starch, or a sugar alcohol such as mannitol. The osmolar component is readily selected by those skilled in the art.

In some embodiments, the preservative is at least one of parabens, chlorobutanol, phenol, sorbic acid, and thimerosal.

In some embodiments, the formulation comprising a compound/agent is in the form of a sustained release formulation and optionally, further comprises one or more additional components, wherein the additional component is at least one of an anti-inflammatory agent; and a preservative.

### Methods of Delivery

Therapeutic compositions comprising an SLC6A8 transporter inhibitor (e.g., β-Guanidinoproprionic acid, RGX-202) and a KRAS inhibitor (e.g., Sotorasib, Adagrasib, MRTX1133, RMC-6291, and RMC-6236) and/or another chemotherapeutic (e.g., FOLFIRI, FOLFOX, bevacizumab, 5-fluorouracil, leflunomide, irinotecan, and/or oxaliplatin) provided herein may be administered via a variety of methods. Such methods include, without limitation, intratumoral, intravesicular, intralesional (e.g., the colon or GI tract), oral, intravenous (iv), subcutaneous (sc or sq), intraperitoneal, intramuscular intradermal, or rectal to a subject (e.g., a mammal) in need thereof. In some embodiments, a sustained release formulation is administered as a suppository. In some embodiments, the sustained release formulation is administered in an implant designed for subcutaneous implantation. Exemplary subcutaneous implants are known to those of skill in the art and may involve a port or catheter or the like. In a particular embodiment, the port or catheter is implanted in the gastrointestinal tract.

### Therapeutic Dosing and Regimen

Combinations featuring an SLC6A8 transporter inhibitor (e.g., β-Guanidinoproprionic acid, RGX-202) and a KRAS inhibitor (e.g., Sotorasib, Adagrasib, MRTX1133, RMC-6291, and RMC-6236) and/or another chemotherapeutic (e.g., FOLFIRI, FOLFOX, bevacizumab, 5-fluorouracil, leflunomide, irinotecan, and/or oxaliplatin) are administered in amounts suitable to increase patient survival, to reduce tumor size, or to otherwise stabilize disease. The therapeutic dosing and regimen best suited for treatment of a subject (e.g., a human patient) vary with the disorder or condition to be treated, and according to the patient's weight and other parameters. A dose of at least one compound/agent described herein may, for example, be administered at about 1000 up to about 3600 mg, twice daily over the course of cancer therapy.

In some embodiments of any of the foregoing methods, the creatine transporter inhibitor, or a pharmaceutically acceptable salt thereof is administered in an amount of 0.01 to 100 mg/kg per day.

Smaller doses, and shorter or longer duration or frequency of treatment, are also envisioned to produce therapeutically useful results, i.e., a statistically significant decrease in cell proliferation and/or tumor size. It is, moreover, envisioned that localized administration to the gastrointestinal tract (e.g., the colon), may be optimized based on the response of gastrointestinal cells therein (e.g., epithelial cells).

An effective dosage and treatment protocol may be determined by conventional means, starting with a low dose in laboratory animals and then increasing the dosage while monitoring the effects, and systematically varying the dosage regimen as well. Numerous factors may be taken into consideration by a clinician when determining an optimal dosage for a given subject, including the size, age, and general condition of the patient, the particular disorder being treated, the severity of the disorder, and the presence of other drugs in the patient. Trial dosages may be chosen after consideration of the results of animal studies and the clinical literature.

A typical human dose of a compound/agent provided herein may be from about 10 μg/kg body weight/day to about 10 mg/kg/day, more particularly from about 50 μg/kg/day to about 5 mg/kg/day, and even more particularly about 100 μg/kg/day to 1 mg/kg/day.

In some embodiments of any of the aspects, the creatine transporter inhibitor (e.g., β-GPA, RGX-202, RGX-202-01), or a pharmaceutically acceptable salt thereof is orally administered in an amount of at least 1000 milligrams (mg) or more, 1200 mg or more, 1400 mg or more, 1600 mg or more, 1800 mg or more, 2000 mg or more, 2200 mg or more, 2400 mg or more, 2600 mg or more, 2800 mg or more, 3000 mg or more, 3200 mg or more, 3400 mg or more, up to about 3600 mg.

In some embodiments of any of the aspects, the creatine transporter inhibitor, or a pharmaceutically acceptable salt thereof is orally administered at least about once per day, twice per day, three times per day, four times per day, or five times per day.

In some embodiments of any of the aspects, the creatine transporter inhibitor, or a pharmaceutically acceptable salt thereof is orally administered in an amount of at least about 2400 mg to about 3600 mg twice per day.

In some embodiments of any of the aspects, the KRAS inhibitor adagrasib or a pharmaceutically acceptable salt thereof is orally administered in an amount of at least about 150 mg or more, at least about 300 mg or more, at least about 600 mg or more, 1400 mg or more, 1600 mg or more, 1800 mg or more, 2000 mg or more, 2200 mg or more, 2400 mg or more, 2600 mg or more, 2800 mg or more, 3000 mg or more, 3200 mg or more, 3400 mg or more, up to about 3600 mg.

In some embodiments of any of the aspects, the KRAS inhibitor sotorasib or a pharmaceutically acceptable salt thereof is orally administered in an amount of at least about 150 mg or more, at least about 300 mg or more, at least about 600 mg or more, 1400 mg or more, 1600 mg or more, 1800 mg or more, 2000 mg or more, up to about 2500 mg.

In some embodiments of any of the aspects, the KRAS inhibitor is MRTX1133 or a pharmaceutically acceptable salt thereof is orally or intravenously administered in an amount of at least about 0.01 mg/kg or more, at least about 1 mg/kg or more, at least about 10 mg/kg or more, at least about 100 mg/kg or more, at least about 300 mg/kg or more, up to about 500 mg/kg,

In some embodiments of any of the aspects, the KRAS inhibitor RMC-6291 or a pharmaceutically acceptable salt thereof is orally or intravenously administered in an amount of at least about 0.01 mg/kg or more, at least about 1 mg/kg or more, at least about 10 mg/kg or more, at least about 100 mg/kg or more, at least about 300 mg/kg or more, up to about 500 mg/kg.

In some embodiments of any of the aspects, the KRAS inhibitor RMC-6236 or a pharmaceutically acceptable salt thereof is orally or intravenously administered in an amount of at least about at least about 0.01 mg/kg or more, at least about 1 mg/kg or more, at least about 10 mg/kg or more, at least about 100 mg/kg or more, at least about 300 mg/kg or more, up to about 500 mg/kg.

In some embodiments of any of the aspects, the KRAS inhibitor (e.g., sotorasib, adagrasib, MRTX1133, RMC-6291, and/or RMC-6236), or a pharmaceutically acceptable salt thereof is orally administered at least about once per day, twice per day, three times per day, four times per day, or five times per day.

In some embodiments of any of the aspects, leucovorin (folinic acid) or a pharmaceutically acceptable salt thereof is orally administered in an amount of at least about at least about 1 mg or more, at least about 5 mg or more, at least about 10 mg or more, at least about 15 mg or more, at least about 20 mg or more, at least about 25 mg or more, at least about 30 mg or more, at least about 35 mg or more, at least about 40 mg or more, at least about 45 mg or more, up to about 50 mg.

In some embodiments of any of the aspects, leucovorin (folinic acid) or a pharmaceutically acceptable salt thereof is intravenously administered in an amount of at least about at least about 100 mg/m² or more, at least about 200 mg/m² or more, at least about 300 mg/m² or more, at least about 400 mg/m² or more, up to about 500 mg/m² or more.

In some embodiments of any of the aspects, irinotecan or a pharmaceutically acceptable salt thereof is intravenously administered in an amount of at least about at least about 100 mg/m2 or more, at least about 200 mg/m² or more, at least about 300 mg/m² or more, at least about 400 mg/m² or more, up to about 500 mg/m².

In some embodiments of any of the aspects, 5-fluorouracil (F) or a pharmaceutically acceptable salt thereof is intravenously administered in an amount of at least about at least about at least about 300 mg/m² or more, at least about 600 mg/m² or more, at least about 900 mg/m² or more, at least about 1200 mg/m² or more, at least about 1500 mg/m² or more, at least about 1800 mg/m² or more, at least about 2100 mg/m² or more, at least about 2400 mg/m² or more, at least about 2800 mg/m² or more, at least about 3000 mg/m² or more, at least about 3300 mg/m² or more, up to about 3600 mg/m².

In some embodiments of any of the aspects, the FOLFIRI (e.g., leucovorin (folinic acid-FOL), 5-fluorouracil (F), and irinotecan hydrochloride (IRI)) is orally and/or intravenously administered at least about once per day, twice per day, three times per day, four times per day, or five times per day.

In some embodiments of any of the aspects, bevacizumab or a pharmaceutically acceptable salt thereof is intravenously administered in an amount of at least about 1 mg/kg or more, at least about 2 mg/kg or more, at least about 3 mg/kg or more, at least about 4 mg/kg or more, at least about 5 mg/kg or more, at least about 7.5 mg/kg or more, at least about 10 mg/kg or more, at least about 15 mg/kg or more, up to about 20 mg/kg or more.

In some embodiments of any of the aspects, bevacizumab or a pharmaceutically acceptable salt thereof is intravenously administered at least about once per day, twice per day, three times per day, four times per day, or five times per day. In some embodiments of any of the aspects, the bevacizumab or a pharmaceutically acceptable salt thereof is intravenously administered at least about every week, at least about every 2 weeks, or at least about every 3 weeks.

Therapeutic efficacy of a compound/agent and/or compositions comprising same may be determined by evaluating and comparing patient symptoms and quality of life pre- and post-administration. Such methods apply irrespective of the mode of administration. In a particular embodiment, pre-administration refers to evaluating patient symptoms and quality of life prior to onset of therapy and post-administration refers to evaluating patient symptoms and quality of life at least 1, 2, 3, 4, 5, 6, 7, 8, 9, 10, 11, 12, 14, 14, 15, 16, 17, 18, 29, 20 weeks after onset of therapy. In a particular embodiment, the post-administration evaluating is performed about 2-8, 2-6, 4-6, or 4 weeks after onset of therapy. In a particular embodiment, patient symptoms (e.g., gastrointestinal upset) and quality of life pre- and post-administration are evaluated clinically and by questionnaire assessment.

### Efficacy of Treatment

Therapeutic combinations featuring an SLC6A8 transporter inhibitor (e.g., (β-Guanidinoproprionic acid, RGX-202) and a KRAS inhibitor (e.g., Sotorasib, Adagrasib, MRTX1133, RMC-6291, and RMC-6236) and/or another chemotherapeutic (e.g., FOLFIRI, FOLFOX, bevacizumab, 5-fluorouracil, leflunomide, irinotecan, and/or oxaliplatin) are useful for treating cancer (e.g., colorectal cancer, gastrointestinal cancer, pancreatic cancer), or any other disease or condition described herein. An effective amount refers to the amount of an active compound/agent that is required to confer a therapeutic effect on a treated subject.

Effective doses will vary, as recognized by those skilled in the art, depending on the types of diseases treated, route of administration, excipient usage, and the possibility of co-usage with other therapeutic treatment.

The compositions and methods provided herein can be used to reduce cancer cell proliferation or survival in vivo or in vitro.

Methods of evaluating tumor progression or cell proliferation are well known in the art.

In some embodiments, overall response is assessed from time-point response assessments (based on tumor burden) as follows:
Complete Response (CR): Disappearance of all target lesions. Any pathological lymph nodes (whether target or non-target) must have reduction in short axis to <10 mm.
Partial Response (PR): At least a 30% decrease in the sum of diameters of target lesions, taking as reference the baseline sum diameters.
Progressive Disease (PD): At least a 20% increase in the sum of diameters of target lesions, taking as reference the smallest sum on study (this includes the baseline sum if that is the smallest on study). In addition to the relative increase of 20%, the sum must also demonstrate an absolute increase of at least 5 mm. (Note: the appearance of one or more new lesions is also considered progression).
Stable Disease (SD): Neither sufficient shrinkage to qualify for PR nor sufficient increase to qualify for PD, taking as reference the smallest sum diameters while on study.

In another embodiment, an in vitro cancer cell proliferation assay is used to assess the efficacy of a creatine transporter inhibitor and/or the one or more additional agent(s) provided herein.

The compositions and methods provided herein result in a reduction in the proliferation or survival of cancer cells. For example, after treatment with one or more of the agents provided herein, cancer cell proliferation or survival is reduced by 5% or greater (e.g., 10%, 20%, 30%, 40%, 50%, 60%, 70%, 80%, 90% or greater) relative to cell proliferation or survival prior to treatment.

The compositions and methods provided herein can result in a reduction in size or volume of a tumor. For example, after treatment, tumor size is reduced by 5% or greater (e.g., 10%, 20%, 30%, 40%, 50%, 60%, 70%, 80%, 90% or greater) relative to its size prior to treatment. Size of a tumor may be measured by any reproducible means of measurement. The size of a tumor may be measured as a diameter of the tumor or by any reproducible means of measurement.

Treating cancer can further result in a decrease in number of tumors. For example, after treatment, tumor number is reduced by 5% or greater (e.g., 10%, 20%, 30%, 40%, 50%, 60%, 70%, 80%, 90% or greater) relative to number prior to treatment. Number of tumors may be measured by any reproducible means of measurement. The number of tumors may be measured by counting tumors visible to the naked eye or at a specified magnification (e.g., 2×, 3×, 4×, 5×, 10×, or 50×).

Treating cancer can result in a decrease in number of metastatic nodules in other tissues or organs distant from the primary tumor site. For example, after treatment, the number of metastatic nodules is reduced by 5% or greater (e.g., 10%, 20%, 30%, 40%, 50%, 60%, 70%, 80%, 90% or greater) relative to number prior to treatment. The number of metastatic noduless may be measured by any reproducible means of measurement. The number of metastatic nodules may be measured by counting metastatic nodules visible to the naked eye or at a specified magnification (e.g., 2×, 10×, or 50×).

Treating cancer can result in an increase in average survival time of a population of subjects treated according to the present invention in comparison to a population of untreated subjects. For example, the average survival time is increased by more than 30 days (more than 60 days, 90 days, 120 days or longer). An increase in average survival time of a population may be measured by any reproducible means. An increase in average survival time of a population may be measured, for example, by calculating for a population the average length of survival following initiation of treatment with the compound of the invention. An increase in average survival time of a population may also be measured, for example, by calculating for a population the average length of survival following completion of a first round of treatment with the compound of the invention.

Treating cancer can also result in a decrease in the mortality rate of a population of treated subjects in comparison to an untreated population. For example, the mortality rate is decreased by more than 2% (e.g., more than 5%, 10%, 25%, or greater). A decrease in the mortality rate of a population of treated subjects may be measured by any reproducible means, for example, by calculating for a population the average number of disease-related deaths per unit time following initiation of treatment with the compound of the invention. A decrease in the mortality rate of a population may also be measured, for example, by calculating for a population the average number of disease-related deaths per unit time following completion of a first round of treatment with the compound of the invention.

Methods of Characterizing a Cancer and Selecting Subjects for Treatment with a Creatine Transporter Inhibitor

The present invention features methods of administering personalized cancer therapy to a subject that has or is at risk of developing cancer. The methods comprise characterizing the KRAS, HRAS, NRAS, CKB, and/or SLC6A8 expression levels, activity, or sequence in a tumor and selecting the subject for treatment when the tumor comprises one or more of: (1) a KRAS, HRAS, and/or NRAS mutation; (2) an increase in the level or activity of creatine, CKB; and/or (3) an increase in the level or activity of SLC6A8, where increases are measured relative to the level, activity, or sequence of KRAS, CKB, and/or SLC6A8 in a corresponding control cell (e.g., a colorectal cell not affected by cancer).

Accordingly, this disclosure provides for the characterization of a biological sample from a subject having or suspected of having cancer (e.g., colorectal cancer). Such characterization includes characterizing a KRAS, HRAS, and/or NRAS polynucleotide sequence in a biological sample obtained from a subject and detecting the presence or absence of an alteration in the KRAS, HRAS, and/or NRAS polynucleotide sequence relative to a reference sequence, wherein detection of an alteration in the KRAS, HRAS, and/or NRAS polynucleotide sequence selects the subject for treatment with a therapeutic described herein, e.g., a creatine transporter inhibitor, a KRAS inhibitor, and/or another chemotherapeutic.

Other methods for characterizing a biological sample from a subject having or suspected of having cancer (e.g., colorectal cancer) involve detecting the level of a creatine kinase B (CKB) polypeptide or polynucleotide in a biological sample obtained from the subject relative to a reference level, wherein an increase in the level or activity of CKB relative to a reference level, selects the subject for treatment with a creatine transporter inhibitor and a KRAS inhibitor. In some embodiments, a biological sample from a subject having or suspected of having cancer (e.g., colorectal cancer) is characterized for the level of CKB and for the presence or absence of an alteration in a KRAS, HRAS, and/or NRAS polynucleotide sequence.

The KRAS, HRAS, and/or NRAS mutations and CKB levels can be detected, e.g., in tissue and liquid biopsy biological samples. Alterations in a KRAS, HRAS, and/or NRAS polynucleotide sequence may be characterized using any method known in the art (e.g., sequencing, Sanger sequencing, next generation sequencing, primer/probe hybridization, immunohistochemistry, Western blot, ELISA). Alterations in CKB levels are characterized, for example, using immunoassays (e.g., sequencing, Sanger sequencing, next generation sequencing, primer/probe hybridization, immunohistochemistry, Western blot, ELISA) or any other method known in the art.

In some embodiments of any of the aspects, the biological sample is further evaluated for the presence or absence of a tumor marker. Non-limiting examples of tumor markers include carcinoembryonic antigen (CEA), carbohydrate antigen (CA) 19-9 or CA19-9, and carbohydrate antigen 15-3 (CA15-3). Carbohydrate antigen (CA) 19-9 is a type of antigen released by pancreatic cancer cells. An increase in the level of CA19-9 indicates that a subject has a tumor that is growing relative to a reference level (e.g., a subject that does not have cancer). CA 15-3 is a tumor antigen expressed in breast cancers. A high level of CEA relative to a reference level can be a sign of certain types of cancers, including cancers of the colon and rectum, prostate, ovary, lung, thyroid, and/or liver.

In some embodiments of any of the aspects, the biological sample (e.g., a tumor specimen) obtained from the subject is further evaluated for an alteration in the level of CKB and/or SLC6A8.

Methods of characterizing, evaluating, and quantifying the level or activity of a creatine transporter or a creatine kinase are provided herein in the working examples in Example 12. For example, the levels of CKB and/or SLC6A8 can be evaluated by immunohistochemistry of a biological sample, e.g., a tissue or liquid biopsy.

### Kits

Therapeutic compositions featuring a therapeutic combination, e.g., an SLC6A8 transporter inhibitor (e.g., β-Guanidinoproprionic acid, RGX-202) and a KRAS inhibitor (e.g., Sotorasib, Adagrasib, MRTX1133, RMC-6291, and RMC-6236) and/or another chemotherapeutic (e.g., FOLFIRI, FOLFOX, bevacizumab, 5-fluorouracil, leflunomide, irinotecan, and/or oxaliplatin) can be provided in a kit. In an embodiment, an SLC6A8 transporter inhibitor (e.g., RGX-202) and a KRAS inhibitor can be provided in a kit. In one embodiment, the kit includes (a) a container that contains the therapeutic composition, and optionally (b) informational material. The informational material can be descriptive, instructional, marketing or other material that relates to the methods described herein and/or the use of the agents for therapeutic benefit. In an embodiment, the kit includes also includes an additional therapeutic agent. For example, the kit includes a first container that contains the composition and a second container for the additional therapeutic agent.

The informational material of the kits is not limited in its form. In one embodiment, the informational material can include information about production of the composition, concentration, date of expiration, batch or production site information, and so forth. In one embodiment, the informational material relates to methods of administering the composition, e.g., in a suitable dose, dosage form, or mode of administration (e.g., a dose, dosage form, or mode of administration described herein), to treat a subject in need thereof. In one embodiment, the instructions provide a dosing regimen, dosing schedule, and/or route of administration of the composition or the additional therapeutic agent. The information can be provided in a variety of formats, including printed text, computer-readable material, video recording, or audio recording, or information that contains a link or address to substantive material.

In addition to the composition, the kit can include other ingredients, such as a solvent or buffer, a stabilizer, or a preservative. The composition can be provided in any form, e.g., liquid, dried or lyophilized form, preferably substantially pure and/or sterile. When the agents are provided in a liquid solution, the liquid solution preferably is an aqueous solution. When the agents are provided as a dried form, reconstitution generally is by the addition of a suitable solvent and acidulant. The acidulant and solvent, e.g., an aprotic solvent, sterile water, or a buffer, can optionally be provided in the kit.

The kit can include one or more containers for the composition or compositions containing a KRAS inhibitor and/or a creatine transporter inhibitor provided herein. In some embodiments, the kit contains separate containers, dividers or compartments for the composition and informational material. For example, the composition can be contained in a bottle, vial, or syringe, and the informational material can be contained in a plastic sleeve or packet. In other embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of the agents. The containers can include a combination unit dosage, e.g., a unit that includes both the KRAS inhibitor and the creatine transporter inhibitor in a desired ratio. For example, the kit includes a plurality of syringes, ampules, foil packets, blister packs, or medical devices, e.g., each containing a single combination unit dose. The containers of the kits can be airtight, waterproof (e.g., impermeable to changes in moisture or evaporation), and/or light-tight.

The kit optionally includes a device suitable for administration of the composition, e.g., a syringe or other suitable delivery device. The device can be provided pre-loaded with one or both of the agents or can be empty, but suitable for loading.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### Examples

### Example 1: Oral RGX-202 Suppresses In Vivo Creatine Import and Depletes Cellular Phosphocreatine and ATP Levels

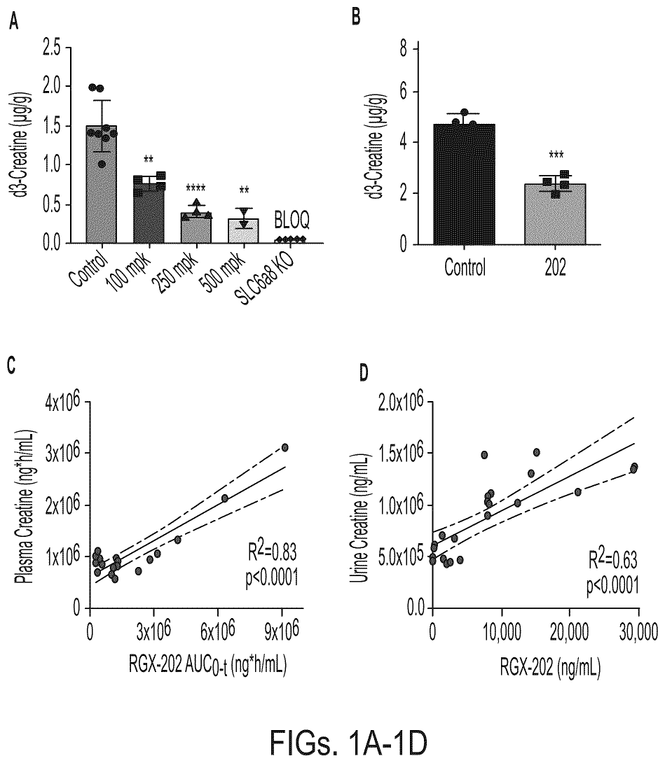

RGX-202 (β-Guanidinoproprionic acid, 3-GPA) is a creatine mimetic that competitively inhibits cellular creatine import by inhibiting the creatine transporter SLC6A8. RGX-202 and its pharmaceutically optimized form RGX-202-01-which is suitable for clinical administration-were used herein to determine the impact of SLC6A8 inhibition on CRC progression. To assess the degree to which RGX-202 inhibits SLC6A8, creatine import was monitored in vivo. Increasing concentrations of RGX-202 were administered, followed by injection of deuterium labeled creatine (d3-creatine) into wild-type and SLC6A8 knock-out mice. Mouse tissue was extracted and analyzed for levels of d3-creatine by LC-Mass Spectroscopy (LC-MS/MS) (23). RGX-202 treatment inhibited tissue uptake of d3-creatine in a dose-dependent manner by up to 75% at 500 mg/kg (FIG. TA). D3-creatine levels in SLC6A8 knock-out animals were below the lower limit of quantification, confirming that creatine import was exclusively mediated by SLC6A8 (FIG. TA). To determine if RGX-202 could inhibit tumoral SLC6A8, similar studies were conducted in mice bearing syngeneic UN-KPC-961 pancreatic tumors. RGX-202 at 800 mg/kg supplemented in the diet for 35 days indeed suppressed tumoral d3-creatine import by 50% (FIG. 1B). These findings revealed that administration of the creatine mimetic RGX-202 significantly suppressed creatine import into tissues in vivo.

When RGX-202-01 inhibits cellular import of creatine, then changes in creatine concentration in the circulation of RGX-202-01-treated mice should be observed. Increasing doses of RGX-202-01 (100, 400 and 1200 mg/kg) were administered and RGX-202-01 was measured in the plasma by LC-MS/MS. An RGX-202-01 exposure-dependent increase in circulating plasma creatine levels was observed, manifested by accumulation of plasma creatine upon blockade of SLC6A8 in tissues (FIG. TC). A substantial exposure-dependent increase in urinary creatine levels was also observed in the same animals (FIG. 1D), consistent with inhibition of creatine import into tissues causing increased creatine in circulation and subsequent excretion in urine, thus reducing creatine levels available to tumors.

### Example 2: SLC6A8 Inhibition Exhibits Broad Anti-Tumor Activity Against Diverse Primary and Metastatic CRCs

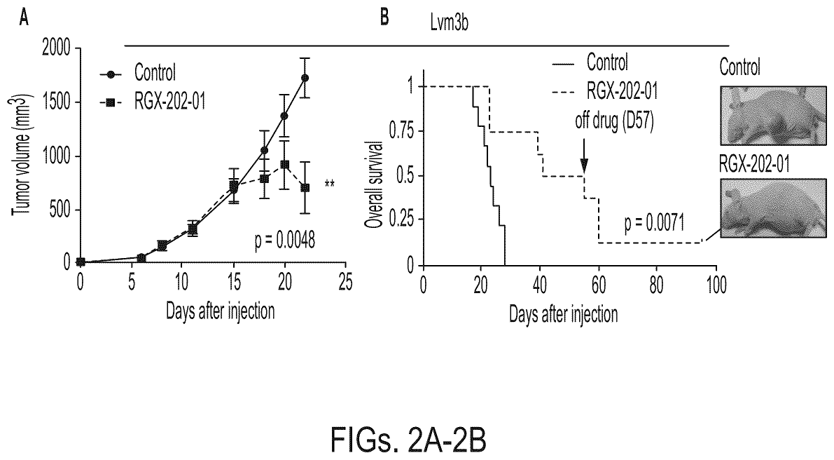

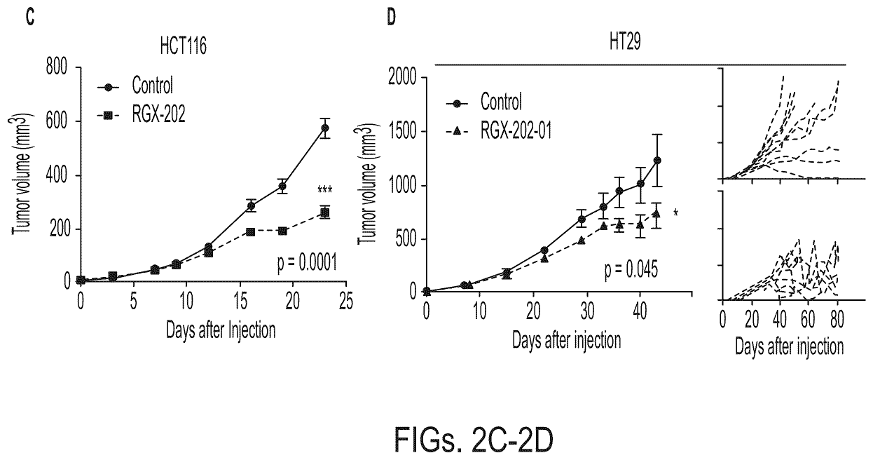

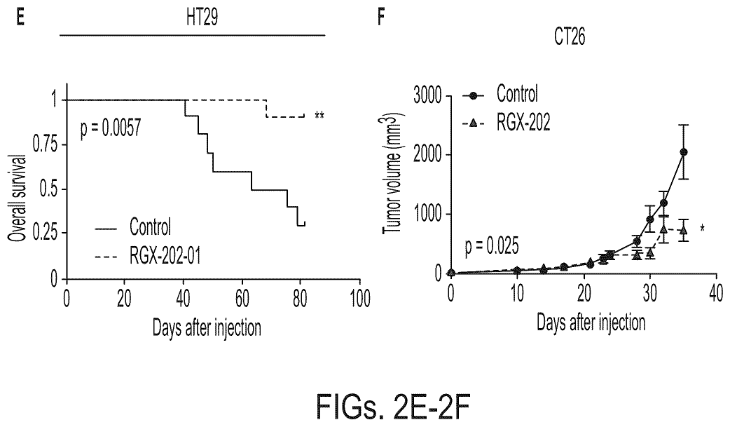

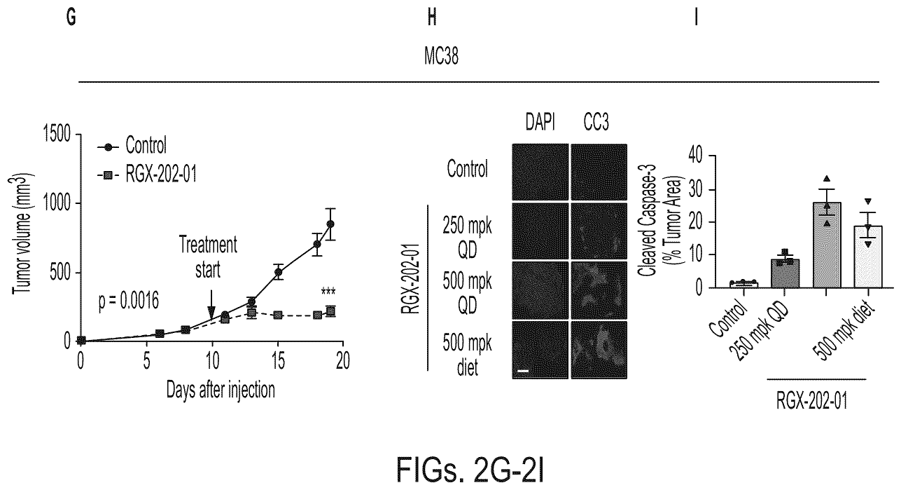

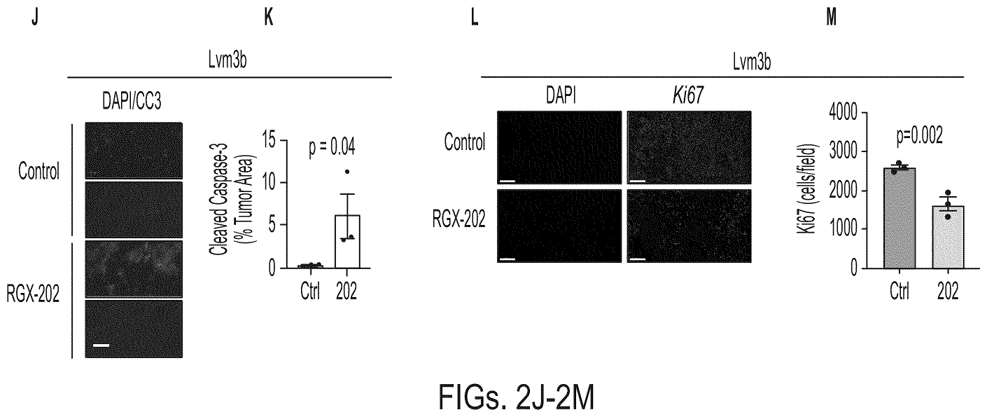

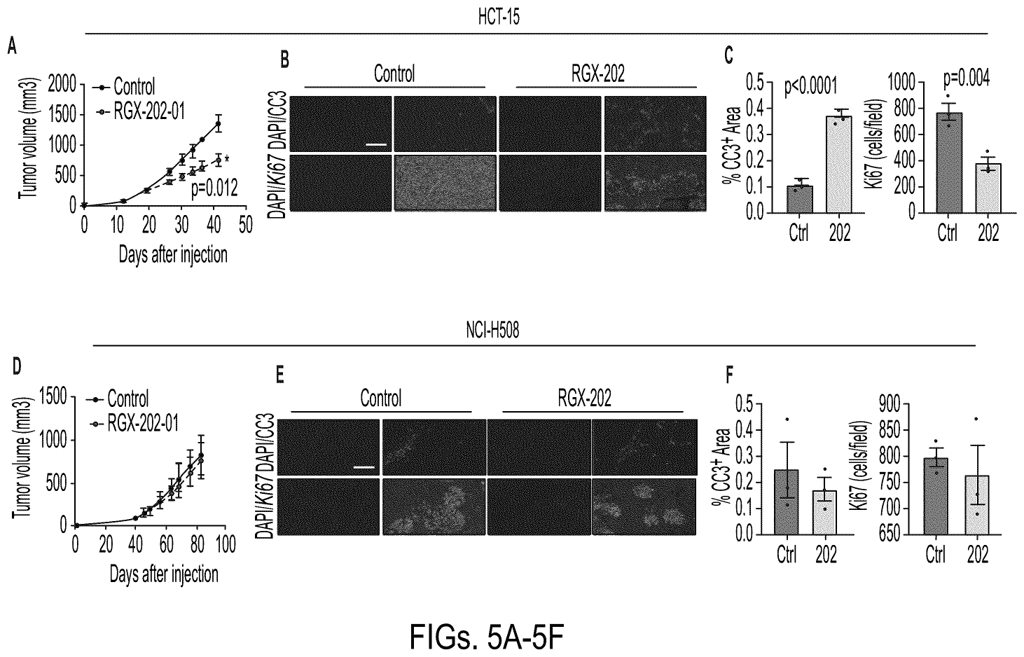

Primary tumors exhibit hypoxia, especially upon progression to larger sizes and must gain metabolic adaptations to the hypoxic microenvironment in order to survive. Approximately 40% of colorectal cancers harbor mutations in the KRAS oncogene. KRAS mutations have been shown to confer increased metabolic demand in cancer cells, including increased dependency on energy producing metabolic pathways. Without intending to be bound by theory, it was hypothesized that these tumors might exhibit sensitivity to the effects of SLC6A8 blockade by RGX-202. To determine whether SLC6A8 inhibition impacts growth of KRAS mutant CRC tumors, highly aggressive metastatic Lvm3b (KRAS G12D) cells were implanted subcutaneously into athymic nude mice and RGX-202-01 treatment commenced after the tumors became palpable (>30 mm³). Oral administration of RGX-202-01 caused a ˜50% tumor growth inhibition (FIG. 2A). Interestingly, the effects of RGX-202-01 emerged after tumors reached larger sizes (>500 mm³), consistent with the previously demonstrated role of creatine metabolism and SLC6A8 in hypoxic survival (Loo et al., supra). RGX-202-01 treated mice exhibited a significant improvement in survival, experiencing a doubling of median survival time from 23 to 48 days. One of the nine immunodeficient mice experienced a complete tumor regression response and the tumor remained undetectable >38 days after treatment termination (FIG. 2B). Anti-tumor efficacy was also observed in HCT116 (KRAS G13D), as well as HT29 (KRAS wild-type) human CRC tumors upon oral RGX-202 (FIG. 2C) or RGX-202-01 administration (FIGS. 2D-2E). Similar to Lvm3b, treatment of HT29 tumors induced regressions in 7 out of 10 mice upon tumors reaching a large size (>900 mm³) and treatment substantially prolonged overall survival (FIGS. 2D and 2E). To determine if SLC6A8 inhibition could suppress progression of murine CRC in an immunocompetent model, KRAS G12D mutant CT26 murine CRC cells were implanted into syngeneic mice. RGX-202-01 significantly inhibited CT26 tumor growth (FIG. 2F). To determine if this approach is effective in treating larger tumors, murine KRAS wild-type MC38 CRC tumors were treated after volumes reached ˜150 mm³. RGX-202-01 administration substantially inhibited tumor growth in this immunocompetent model (FIG. 2G). Drug treatment significantly enhanced in vivo tumor apoptosis in a dose-dependent manner as quantified by cleaved caspase-3 immunohistochemistry (FIGS. 2H and 21). Increased apoptosis was also observed in RGX-202-01-treated Lvm3b and HCT-15 tumors (FIGS. 2J and 2K, and FIGS. 5A-5C). It was next asked whether RGX-202-01 modulates tumor cell proliferation by quantifying Ki67 positive cells. RGX-202 treatment inhibited tumor cell proliferation in both the Lvm3b and HCT15 tumor models (FIGS. 2L and 2M, FIGS. 5B and 5C). In contrast, no effect on apoptosis or proliferation was observed in NCI-H508, a xenograft that did not respond to RGX-202-01 treatment (FIGS. 5D-5F). These results indicated that anti-tumor responses were associated with inhibition of tumor cell proliferation and induction of tumor apoptosis.

### Example 3: SLC6A8 Inhibition Exhibits Broad Activity Against CRC PDX Models

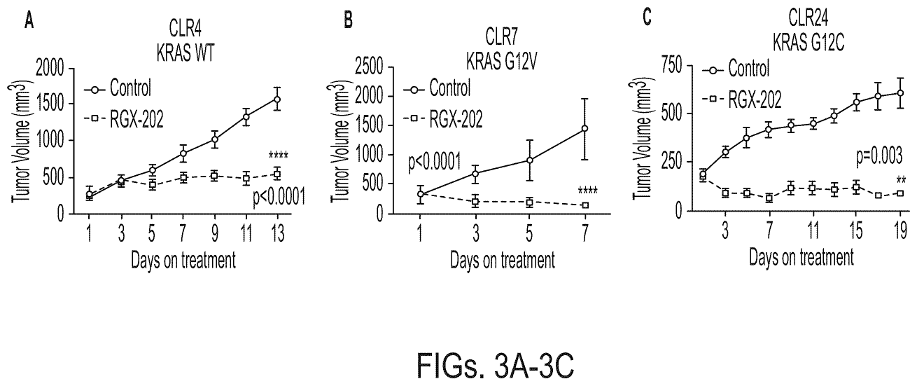

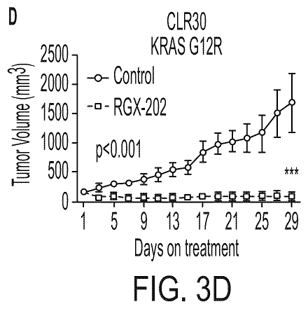

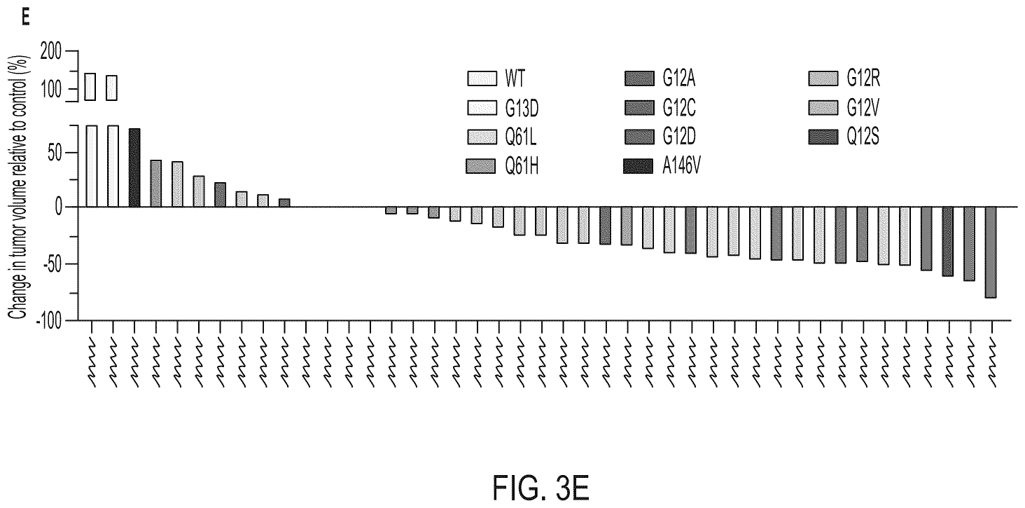

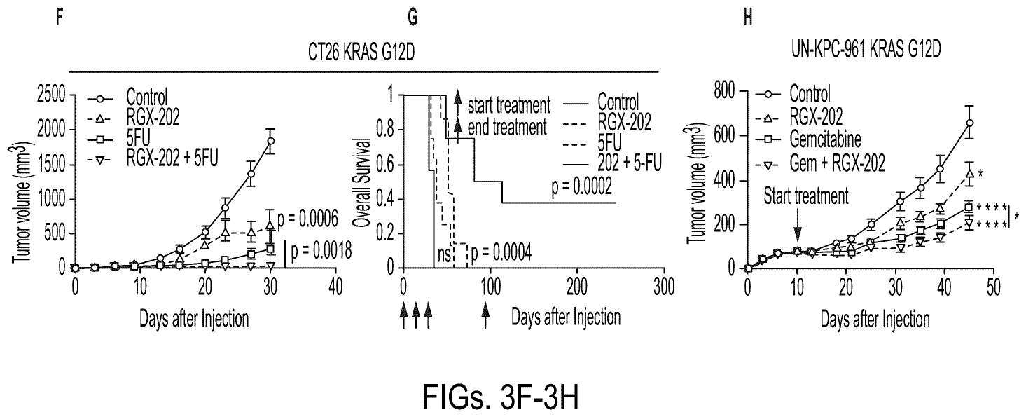

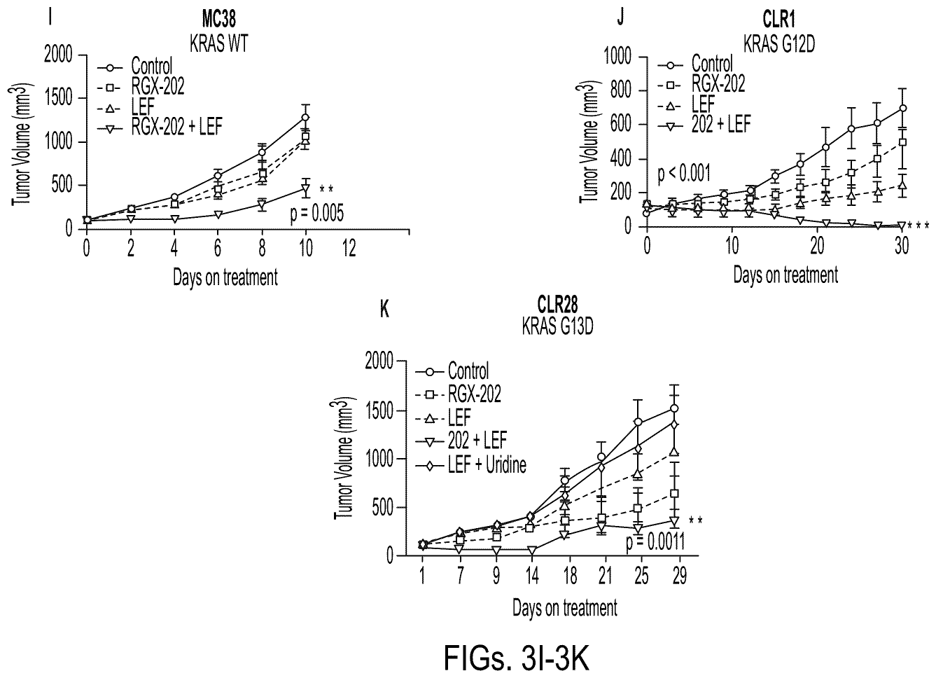

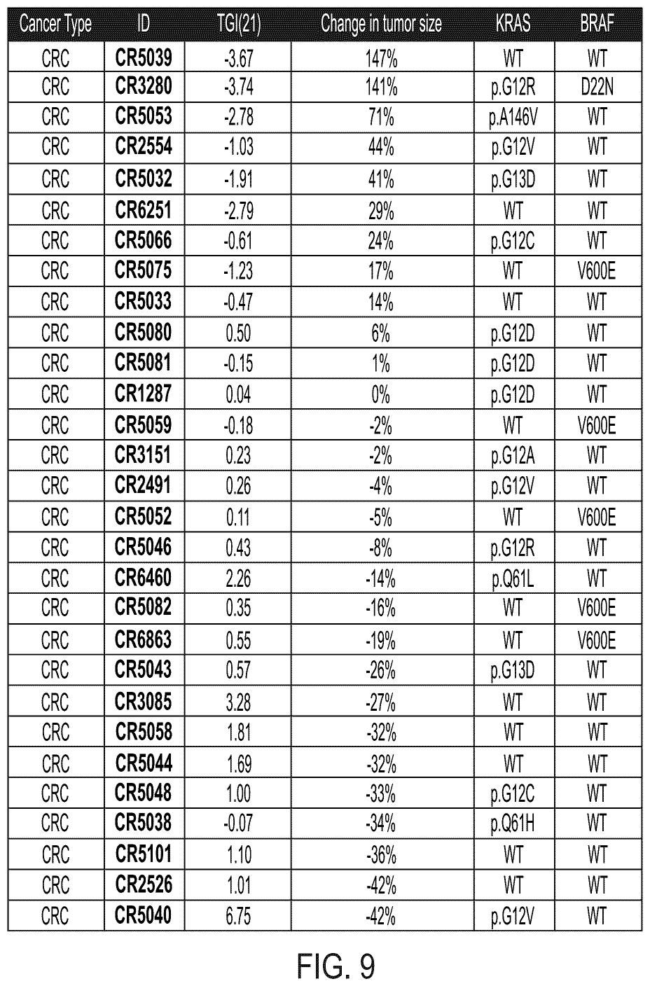

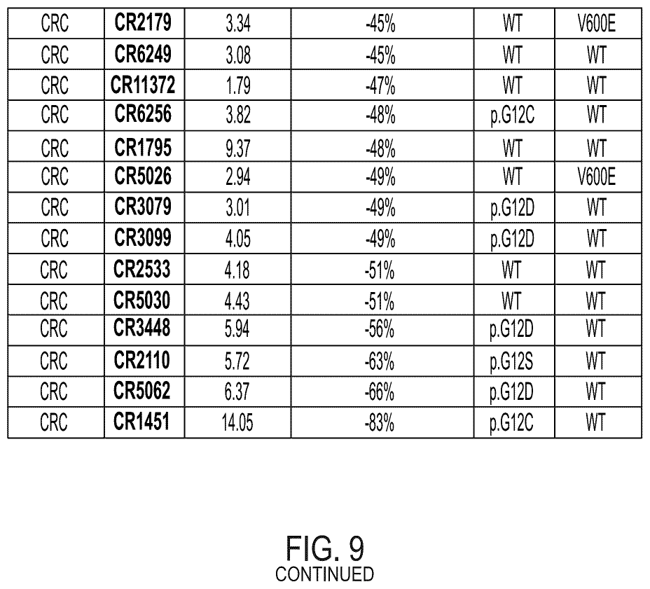

Patient derived xenografts (PDXs) are thought to better recapitulate human tumor pathology and clinical drug responses (27-30). The efficacy of SLC6A8 therapeutic inhibition was assessed on the growth of established (100-250 mm³) human KRAS wild-type and KRAS mutant CRC PDX tumors. Oral RGX-202 administration caused a 65% reduction in the growth of the CLR4 KRAS wild-type PDX model (FIG. 3A). Moreover, treatment also elicited tumor regressions in three PDX models harboring distinct KRAS mutations (FIGS. 3B-3D; CLR7 KRAS G12V, CLR24 KRAS G12C, and CLR30 KRAS G12R). To further characterize the range of efficacy of RGX-202-01 across diverse subtypes of CRC tumors, including KRAS wild-type and KRAS mutant CRC PDX tumors a mouse PDX trial was conducted based on a 1×1 trial design where two mice become inoculated with the same PDX model and one mouse receives RGX-202-01 and the other receives control treatment (31). Such a trial design was developed to mimic a patient clinical trial and has shown reproducibility and translatability of therapeutic responses (31). We included 43 well documented colorectal cancer PDXs of various mutational backgrounds including both KRAS wildtype and KRAS mutant subtypes (FIG. 9). Of these models, 49% of tumors harboring diverse KRAS mutant subtypes exhibited single agent anti-tumor efficacy greater than 30% relative to matched control animals when treated with RGX-202-01 (FIG. 3E). RGX-202 treatment also demonstrated anti-tumor efficacy in 30% of BRAF mutant tumors (2/7) (FIG. 9). These findings reveal that SLC6A8 inhibition by oral RGX-202 treatment exhibits single agent anti-tumor efficacy against a broad range of CRC tumor subtypes and suggests potential for clinical benefit in both KRAS wild-type and KRAS mutant cancers.

### Example 4: SLC6A8 Inhibition Synergizes with 5-FU and Oral Leflunomide Therapy

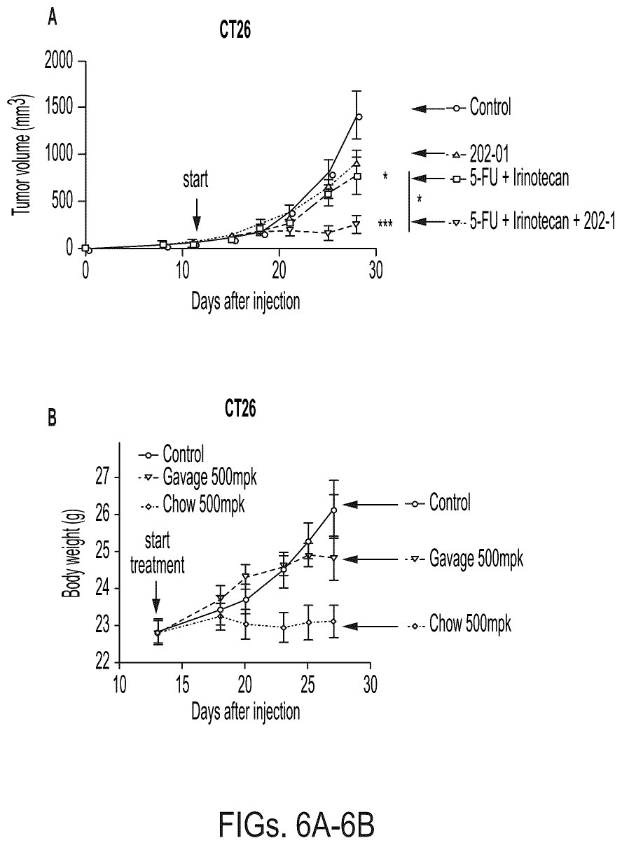

It was next determined whether SLC6A8 inhibition could cooperate or synergize with other therapeutics. 5-FU is the backbone of many chemotherapeutic regimens used in CRC. CT26 subcutaneous tumors were treated in immunocompetent syngeneic mice with vehicle control, oral RGX-202, 5-FU, or a combination regimen comprising RGX-202 and 5-FU. While single-agent RGX-202 or 5-FU significantly suppressed tumor growth (66% inhibition RGX-202, 85% inhibition 5-FU), combined RGX-202/5-FU caused a 99% tumor growth reduction (FIG. 3F). Importantly, survival studies revealed that combined RGX-202/5-FU therapy dramatically enhanced survival of mice, causing 40% of mice to experience complete regression responses and long-term (>240 days) survival (FIG. 3G) despite cessation of treatment at day 85, 160 days prior to termination of the study. RGX-202 also elicited enhanced anti-tumor efficacy in combination with 5-FU and irinotecan, a standard of care regimen for metastatic colorectal cancer (FIG. 6A). Combined treatment of established UN-KPC-961 (Kras^{G12D};Trp53^{R172H}) pancreatic cancer tumors with RGX-202 and gemcitabine, a deoxycytidine analog and an approved pancreatic cancer therapeutic, elicited greater tumor suppression relative to either gemcitabine or RGX-202 alone, though the enhanced effects were modest compared to combination effects observed in CRC (FIG. 3H). It was previously shown that under hypoxia, the growth of metastatic CRC tumors is highly dependent on pyrimidine nucleotide biosynthetic pathways and that these cells become sensitized to inhibition of the dihydroorotate dehydrogenase (DHODH) enzyme, which catalyzes a critical step during pyrimidine nucleotide biosynthesis. Treatment of mice with the oral DHODH inhibitor leflunomide, which is used as a rheumatoid arthritis drug, inhibited CRC primary tumor and metastatic progression. Combined treatment of established MC38 KRAS wild-type syngeneic CRC tumors with RGX-202/leflunomide elicited synergistic activity relative to single-agent treatment with either drug (FIG. 3I). We then tested CLR1 KRAS G12D mutant and CLR28 KRAS G13D mutant PDXs, which exhibited similar synergistic tumor regressions upon combined administration of RGX-202/leflunomide relative to tumor growth suppression responses by either agent alone (FIGS. 3J-3K). To determine if the effect of leflunomide on tumor growth repression was secondary to pyrimidine nucleotide depletion, we included a cohort in the CLR28 model that received leflunomide and Uridine, a pyrimidine nucleotide. Uridine supplementation rescued tumor growth inhibition by leflunomide, consistent with pyrimidine nucleotide levels being limiting for tumor growth (FIG. 3K).

RGX-202 treated animals did not show any adverse effects except for lack of body weight gain in animals that were fed a RGX-202-formulated diet but not when mice received treatment by oral gavage (FIG. 6B). Overall, our findings reveal that RGX-202 is well tolerated as an oral therapy and can cooperate or synergize with the standard of care agent 5-FU as well as the FDA approved rheumatologic oral drug leflunomide.

### Example 5: Tumoral CKB Expression as a Predictive Biomarker of SLC6A8 Inhibition Response

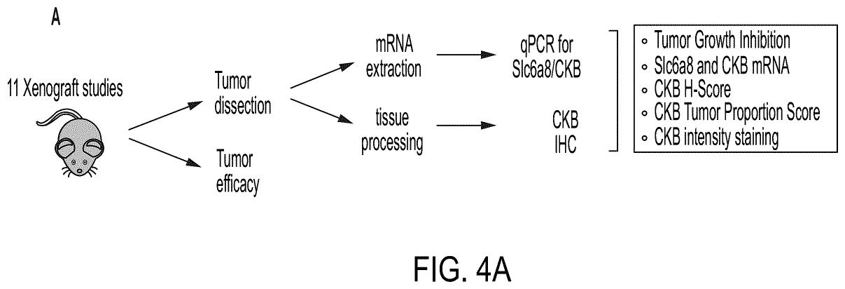

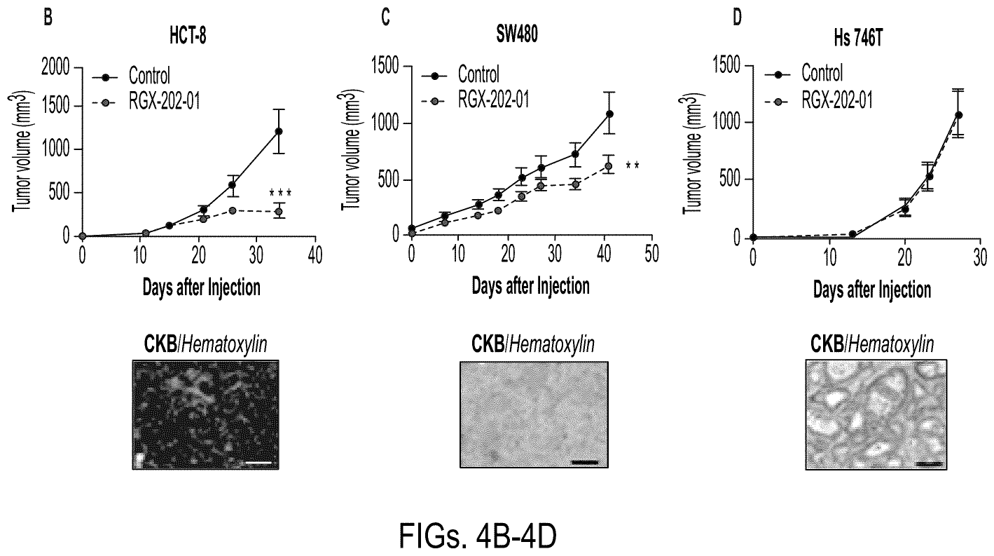

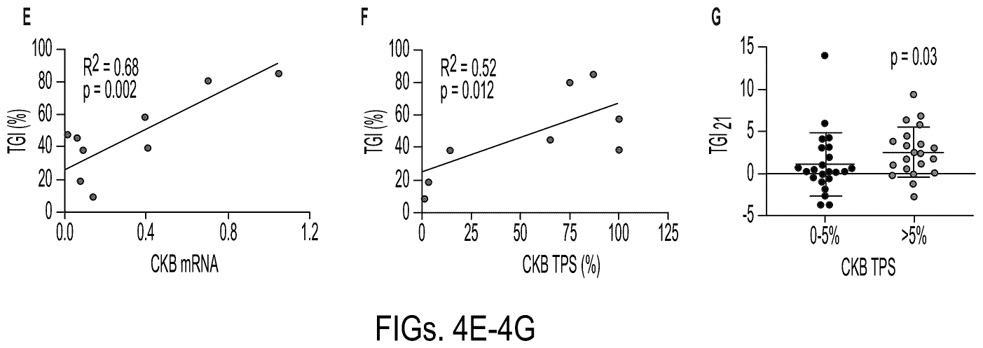

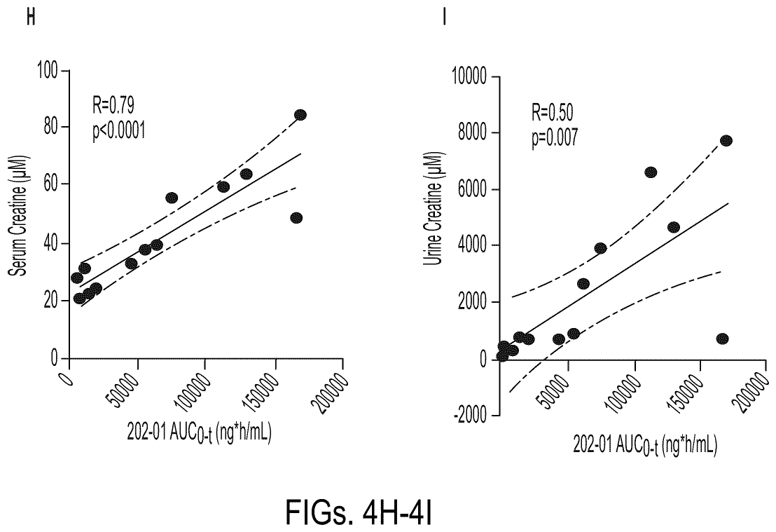

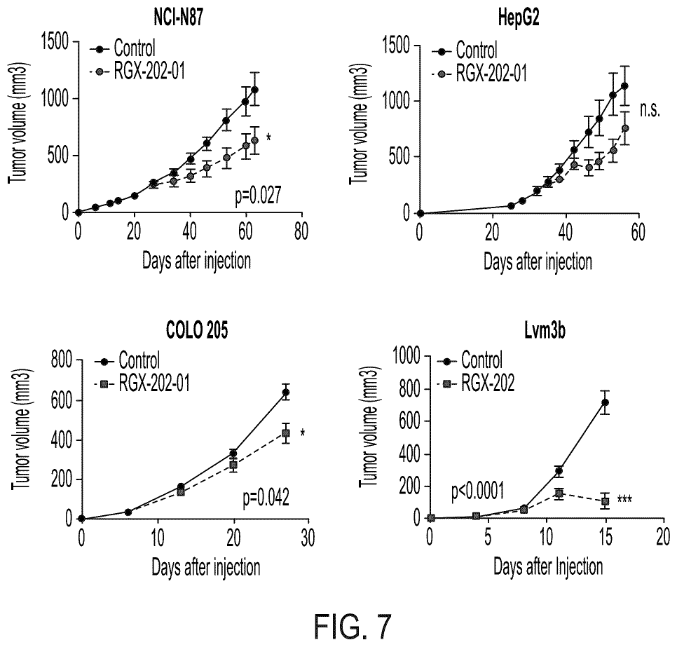

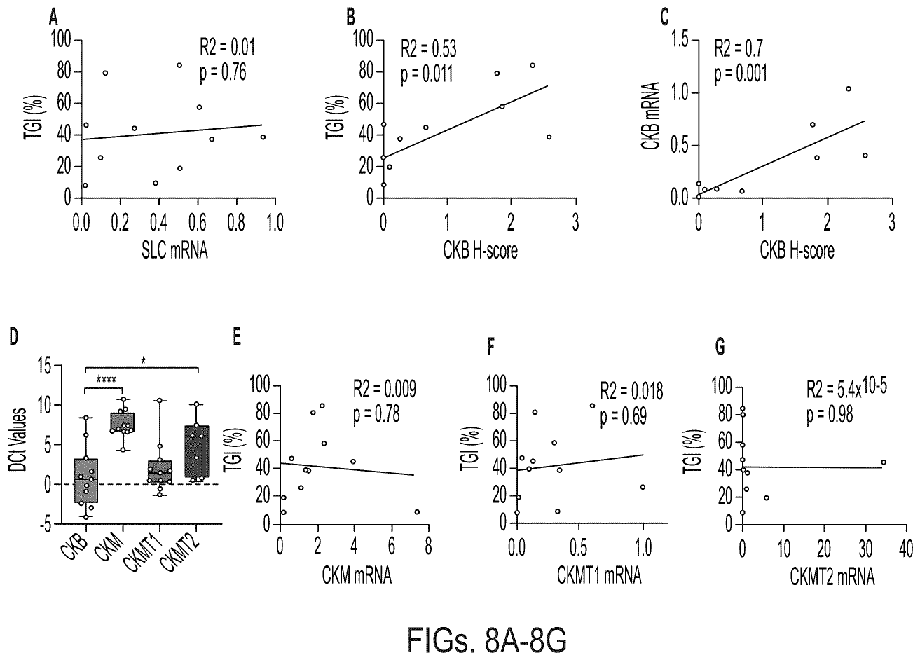

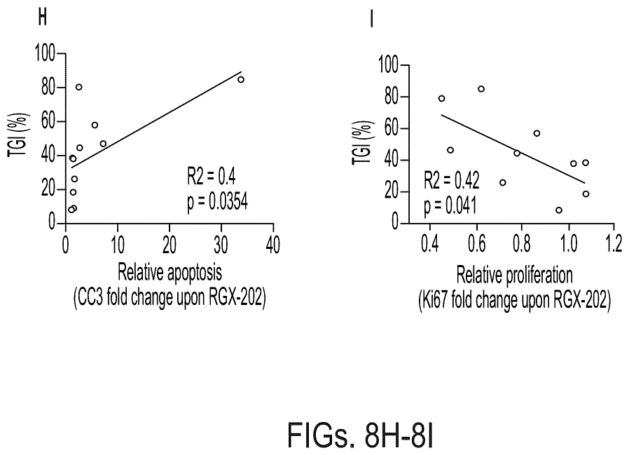

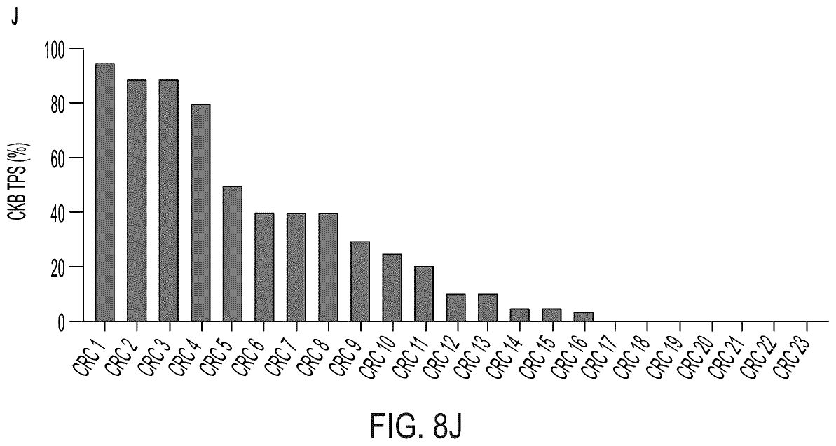

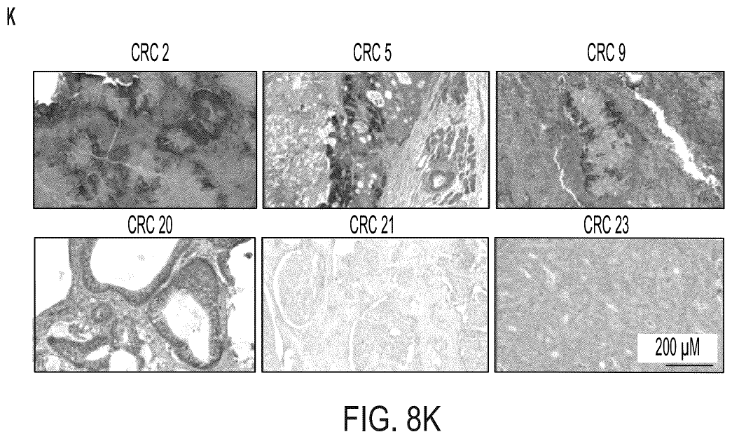

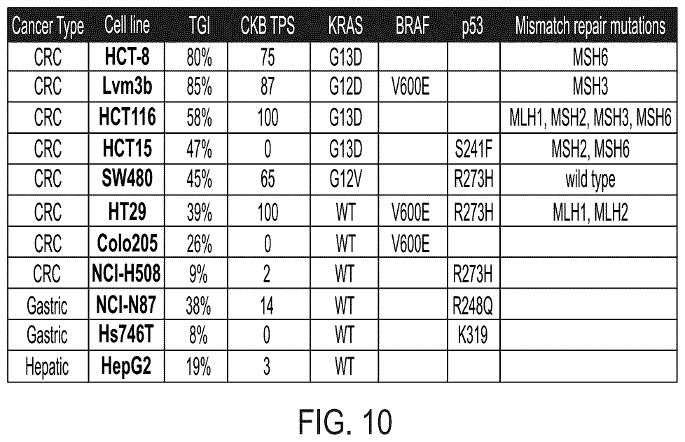

To identify a predictive biomarker for therapeutic response to SLC6A8 inhibition by RGX-202, the following experiments were undertaken. Therapeutic efficacy was observed upon SLC6A8 inhibition in both KRAS mutant, KRAS wildtype, and mismatch repair mutant CRC lines suggested that sensitivity to SLC6A8 inhibition was not solely attributable to oncogenic or genomic instability mutational backgrounds (FIGS. 9, 10). It was previously showed that the CKB enzyme is released from CRC cells and acts upstream of the SLC6A8 transporter by generating the energetic metabolite phosphocreatine, which is imported through the SLC6A8 transporter (Loo et al., supra). Extracellular phosphocreatine supplementation rescued hypoxic survival impairment caused by CKB depletion in an SLC6A8-dependent manner (Loo et al., supra). CKB expression was also shown to be repressed by microRNA-mediated silencing, raising the possibility that variation in its expression may predict sensitivity to therapeutic targeting of this axis (Loo et al., supra). A collection of gastrointestinal cell lines as xenografts was tested for responsiveness to RGX-202-01 (FIG. 10 for details on the cell lines). An independent cohort of animals were simultaneously inoculated with the same xenografts and tumors were allowed to reach ˜ 500 mm³, at which point tumoral SLC6A8 and CKB gene expression were assessed by qPCR and CKB protein expression by immunohistochemistry (IHC) (FIG. 4A). SLC6A8 was not tested by IHC due to lack of availability of an SLC6A8-specific antibody. Included in this assessment were tumors that responded to treatment and those that did not (FIGS. 4B-4D, FIG. 10 and FIG. 7). Therapeutic responses to RGX-202-01 positively correlated with increased CKB mRNA expression (FIG. 4E) but not SLC6A8 mRNA expression (FIG. 8A). Consistent with this, the extent of CKB protein expression-as assessed by Tumor Proportion Score (TPS)—also positively correlated with the magnitude of therapeutic response (FIG. 4F). Tumors expressing elevated CKB protein levels (FIG. 4B) exhibited greater tumor growth repression responses relative to those expressing reduced CKB levels (FIG. 4D and FIGS. 8B and 8C). To determine if this observation could be recapitulated in an independent dataset, the association of CKB protein expression with therapeutic responses in the aforementioned mouse RGX-202-01 1×1 PDX trial was assessed. A similar predictive association of elevated CKB protein expression by IHC with anti-tumor response was observed (FIG. 4G). Creatine kinases are dimeric enzymes that consist of two subunits, CKB (brain type) or CKM (muscle type). Three different isoenzymes therefore exist: CK-MM, CK-BB and CK-MB. Studies were undertaken to determine whether any of the other isoforms might be contributing to efficacy and measured CKM expression in tumors by qPCR. Analysis of the 11 aforementioned xenografts revealed significantly lower expression of CKM compared to CKB (FIG. 8D) and no correlation of CKM expression with anti-tumor efficacy was observed (FIG. 8E). Expression of the two mitochondrial creatine kinases CKMT1 and CKMT2 was also assessed. While both enzymes were detectable, expression of CKMT2 was significantly lower than CKB expression with 4/11 xenograft having undetectable CKMT2 expression. No association of CKMT expression with response to RGX-202 treatment was observed FIGS. 8D, 8F and 8G). These results demonstrate that elevated tumoral CKB mRNA and protein expression predict for enhanced responsiveness to SLC6A8 inhibition by RGX-202-01, consistent with CKB being an upstream component of the CKB/SLC6A8 phosphocreatine metabolic axis in CRC and a gene that exhibits post-transcriptional expression modulation and variation in cancer. Consistent with the proposed mechanism of action of RGX-02-01, a positive correlation of response to RGX-202-01 was observed with induction of cleaved caspase-3, a marker of apoptosis, and a negative correlation of response to RGX-202-01 with the proliferation marker Ki67 (FIGS. 8H-8I).

To assess what fraction of patients with metastatic CRC harbor tumors with elevated CKB protein expression, 23 human metastatic CRC specimens were immunohistochemically stained for CKB. CKB positivity (TPS >5%) was observed in the majority (56%; 13/23) of the tumor tissue samples. Overall, these findings reveal CKB as a patient stratification biomarker for clinical development of SLC6A8 inhibitors. CKB expression in a tissue microarray containing specimens from 10 human tissues was next evaluated. The highest CKB expression with a 100% TPS was observed in the brain and heterogenous expression patterns in several tissues including colon, appendix, bladder, kidney, myometrium and pancreas.

### Example 6: RGX-202-01 Increases Serum and Urinary Creatine Excretion in Patients with Cancer

Motivated by the robust pre-clinical efficacy of RGX-202-01, a multicenter Phase 1a/b clinical trial in patients with advanced gastrointestinal cancers that had progressed on standard of care regimens (ClinicalTrials.gov, NCT03597581) was initiated. In the Phase 1a dose escalation stage of the study, patients received oral RGX-202-01 treatment at doses ranging from 600 mg to 3600 mg twice daily, on a continuous regimen. Blood and urine samples were collected from 13 patients on Day 15 during the first cycle (28 days), and bioanalytic analyses were conducted by an independent contract research laboratory using commercially available proprietary assays. Creatine concentrations in both serum and urine showed positive correlations with systemic exposure to RGX-202-01 (FIGS. 4H and 41). These findings provide proof-of-concept for therapeutic targeting of creatine metabolism in humans, mirroring experimental observations in mice.

The creatine/phosphocreatine bioenergetic shuttle is a critical system that allows highly metabolic tissues to respond to energetic stress quickly through the generation of high energy ATP in a reaction that does not require oxygen. CRC and pancreatic cancers are particularly hypoxic malignancies. The ability of such cancers to over-express and release CKB as a means of generating extracellular phosphocreatine for import through the SLC6A8 transporter enables cancer cells to enhance availability of high energy phosphate for ATP generation in the context of hypoxic and metabolic stresses. SLC6A8 inhibition by RGX-202-01 reduced tumor growth in numerous syngeneic, xenograft and PDX mouse models. Anti-tumor efficacy was associated with enhanced tumor apoptosis and reduced tumor cell proliferation, consistent with prior findings supporting a critical role for creatine metabolism in CRC progression and hypoxic survival (Loo et al., supra). These results provide evidence revealing that RGX-202-01 targets the SLC6a8 creatine/phosphocreatine axis. Without intending to be bound by theory, it is possible that RGX-202-01 can mediate additional anti-tumor effects via additional target(s).

This work reveals that mutational background is not a significant predictor of response to SLC6A8 inhibition and that RGX-202-01 impairs the growth of a broad set of CRC tumors of distinct KRAS and mismatch repair mutant backgrounds suggesting that rather diverse CRC tumors exploit creatine/phosphocreatine metabolism to drive progression. Consistent with a critical role for CKB in this pathway, CKB expression levels were associated with response to RGX-202. Tumors with high CKB expression responded better than those with reduced levels, suggesting that CKB over-expressing tumors are more dependent on phosphocreatine as an energy source and thus more sensitive to its depletion. These findings support the use of patient tumor CKB expression as a molecular biomarker for patient stratification in clinical trials.

These data also reveal that inhibition of SLC6A8 and blockade of cellular creatine/phosphocreatine uptake leads to increased creatine excretion in the urine, both in mice and in patients. Urinary creatine levels positively associated with blood RGX-202-01 concentrations and represent a direct measure of SLC6A8 inhibition. The opportunity for non-invasive monitoring of the pharmacodynamics of creatine transport inhibition through urinalysis of creatine and potentially additional metabolites, provides a simple and rapid method for assessing target engagement in patients.

These findings reveal both single-agent activity and combination efficacy of RGX-202-01 in CRC. Because 5-FU is the mainstay chemotherapeutic used in CRC, the activity of RGX-202 in combination with 5-FU and 5-FU/irinotecan was tested. The data showed synergistic effects of SLC6A8 inhibition and complete tumor regressions with these standard of care regiments. Collectively, these results provide a rationale for incorporating RGX-202-01 into combination regimens including standard of care agents or those targeting nucleotide synthesis.

Overall, this work supports the therapeutic targeting of creatine metabolism in CRC through the inhibition of the SLC6A8 transporter. This treatment approach is currently being tested in a multi-center national Phase 1b/2 clinical trial in CRC (ClinicalTrials.gov, NCT03597581).

### Example 7: RGX 202-01+FOLFIRI Dose Escalation Clinical Trial

RGX 202-01 monotherapy was shown to be safe and effective in subjects with gastrointestinal (GI) adenocarcinoma. To determine whether RGX 202-01 could be effective in combination with a conventional therapy for colorectal cancer, FOLinic acid-Fluorouracil-IRInotecan regimen (FOLFIRI), a combination dose escalation of RGX-202-01 was tested with FOLFIRI. Eleven patients were enrolled, including gastrointestinal (GI) adenocarcinoma patients (median prior lines of therapy). Dose levels of 1800 mg two times daily (BID), 2400 mg BID, and 3000 mg BID were cleared with no significant adverse events. 600 mg tablets were available in a highly compressed salt form of API.

RGX-202-01 and FOLFIRI was administered to 7 colorectal cancer (CRC) patients. Six of the seven evaluable patients (85.7%) had stable disease. The median duration of treatment (PFS surrogate) was −14 weeks. The patients had a median progression-free survival (PFS) of only −8 weeks with FDA approved TAS-102 and Regorafenib in late-line CRC patients.

### Example 8: Clinical Activity in Second Line (2L) CRC with RGX 202-01+FOLFIRI/Bevacizumab

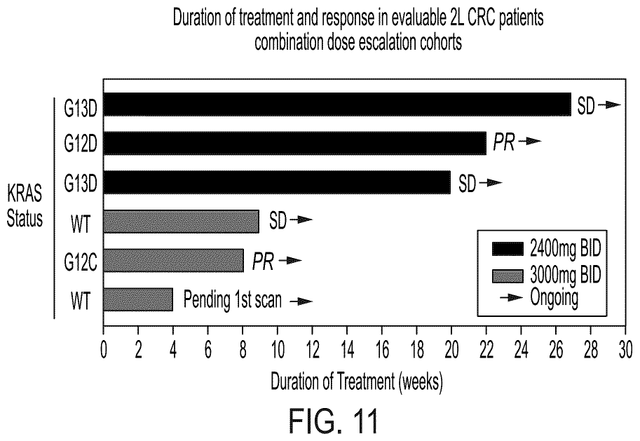

Eight patients with colorectal cancer were enrolled into the second line dose escalation cohorts and provided with 2400 mg or 3000 mg RGX-202-01 two times daily (BID). Five patients remained on therapy (FIG. 11). One patient was pending their first efficacy assessment and 2 patients were not evaluable (off treatment). 40% (2 of 5) of the patients had a partial response to treatment. 60% (3 of 5 patients) were disease-stable and none of the patients had progressive disease. This result was promising, particularly given that the historical overall response rate with FOLFIRI+bevacizumab in 2L CRC is ˜5-10% with median progression-free survival ˜5-6 months (see e.g., Bennouna et al., Lancet Oncol 2013; 14:29 37).

### Example 9: Confirmed Partial Response (PR) in Second Line CRC Patient Treated with 2400 Mg Two Times Daily of RGX 202-01+FOLFIRI/Bevacizumab

Patient 2408 in the second line trial (Example 8) was further evaluated and confirmed to be a partial responder to combination therapy with RGX 202-01 and FOLFIRI/Bevacizumab. Patient 2408 was a 52 year old male with metastatic KRAS G12D mutant colorectal cancer (CRC). Prior to therapy the patient was refractory to oxaliplatin capecitabine with progressive disease. Response evaluation criteria in solid tumors (RECIST), which is a set of published rules that define when tumors in cancer patients improve, was used to measure how well the cancer patient responds to treatment. It is based on whether tumors shrink, stay the same, or get bigger. To use RECIST, there must be at least one tumor that can be measured on x-rays, CT scans, or MRI scans. The types of response a patient can have are a complete response (CR), a partial response (PR), progressive disease (PD), and stable disease (SD). Patients in RECIST 1.1 were evaluable for response if they had measurable disease and at least one follow up scan with at least one cycle of treatment.

At end of cycle Patient 2408 had a 230% reduction in target lesions (multiple liver and lung metastases). A confirmatory scan was completed 4 weeks later for to confirm partial response to treatment with a 310% reduction in tumor size. At the end of the C4 scan showed stable disease with 19% tumor growth from nadir (i.e., the local minimum relative tumor-size change value).

### Example 10: PR in 2L CRC Patient Treated with 3000 mg Twice Daily of RGX 202-01+FOLFIRI/Bevacizumab

Patient 105-2501 was another partial responder. Patient 105-2501 was a 57 year old female with metastatic KRAS G12C mutant colorectal cancer (CRC). Patient was previously treated with oxaliplatin capecitabine and stable disease was the best response. Partial response by RECIST 1.1 was observed in patient 105-2501 on first scan at end of cycle 2. A 31% reduction in target lesions (multiple abdominal wall metastases) were observed.

### Example 11: Clinical Activity Observed in RGX-202-01 Monotherapy and Combination Dose Escalation Cohorts

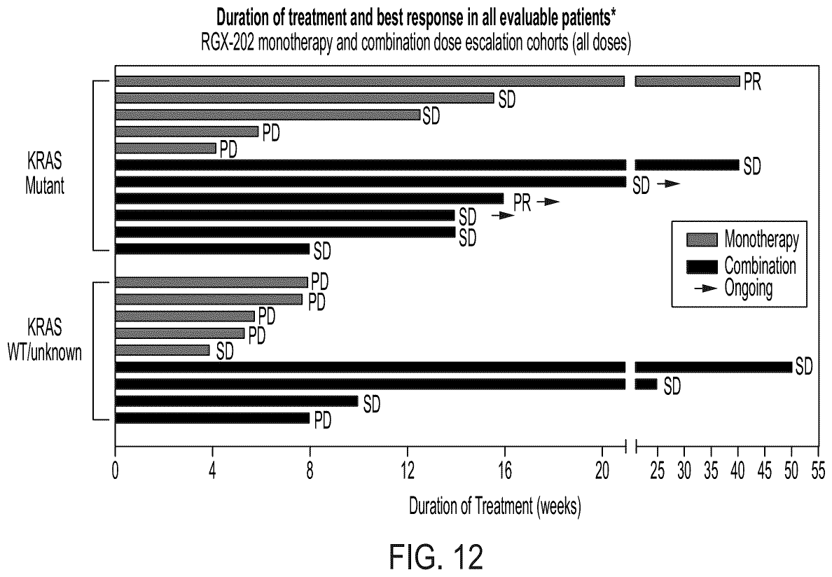

Patients enrolled onto the monotherapy and combination dose escalation cohorts were highly refractory and not pre-selected based on CKB status (no requirement to test for CKB positivity for enrollment onto dose escalations). Clinical response was observed in CRC patients receiving ≥2400 mg BID of RGX-202-01 as a monotherapy or in combination with chemotherapy. Twenty total mono/combo patients were evaluable for response. Four evaluable patients were at doses ≥2400 mg two times daily of RGX 202. Of the 9 KRAS mutant patients evaluated, 2 had partial response (PR) (22%), 6 had stable disease (SD) (67%), and 1 had progressive disease (PD) (11%) when treated with to RGX-202-01 mono and combination therapy. Of the 5 KRAS WT/unknown patients, 0 were partial responders (0%), 1 had stable disease (20%), and 4 still exhibited progressive disease (PD) (80%) to RGX-202-01 mono and combination therapies. Therefore, the greatest clinical benefit was observed in KRAS mutant patients in both monotherapy and combination cohorts (FIG. 12).

### Example 12: RGX-202-01 Activity in Murine PDX Models is Associated with Tumor CKB Expression

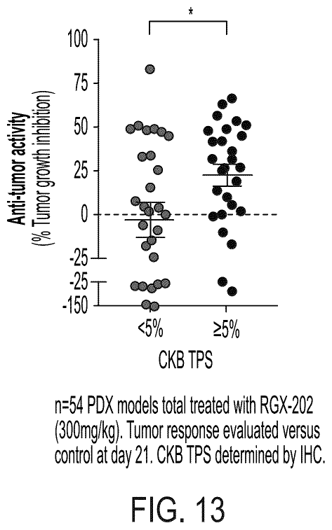

CKB expression (by tumor immunohistochemistry) was assessed in a human Patient Derived Xenograft (PDX) models. Tumor response data for the same models were evaluable for 54 of the 60 PDX animals-colorectal cancer (CRC) (n=43), gastric (n=6), and pancreatic cancer (n=5). Significantly greater RGX-202 therapeutic efficacy activity was observed in CKB positive PDX models (cutoff of ≥5% tumor proportion score (TPS)) versus CKB negative models (<5% TPS) (FIG. 13).

### Example 13: RGX-202-01 has Activity in CRC and Gastric Cancer PDX Murine Models

The therapeutic activity of RGX-202-01 was observed in various genetic subtypes of colorectal cancer (CRC) and gastric cancer PDX models including:
1) KRAS wild type and mutant CRC (G12C, G12D, G12S, and G12V);
2) KRAS wild type and mutant gastric cancer;
3) HER2+(mutant and amplified) CRC and gastric cancer; and
4) Activity in PDX models previously treated with chemotherapy and/or EGFR inhibitors.

### Example 14: Pharmacokinetic and Pharmacodynamic Data for RGX-202-01 and FOLFIRI

Pharmacokinetic and pharmacodynamic data was obtained from 28 subjects treated with RGX-202, either as monotherapy or in combination with FOLFIRI across multiple dose escalation cohorts. Systemic exposure to RGX-202-01 was slightly greater than dose proportional, both in monotherapy and in combination with FOLFIRI. Steady state was achieved by Day 15 of Cycle 1.

The effects of RGX-202-01 on creatine metabolism were monitored by measuring creatine, creatinine, guanidinoacetate and creatine phosphokinase levels in urine and serum/plasma obtained from subjects receiving treatment. An RGX-202-01-dose dependent increase in serum and urine creatine levels was observed by Day 15 of Cycle 1. Systemic exposure to RGX-202-01 (area under the curve: AUC) shows a positive correlation with urinary and serum creatine. Patients with partial response or stable disease exhibited a higher and more durable increase in serum and urine creatine concentration than that in patients with progressive disease.

CKB protein expression in tumor specimens from patients was assessed using a Clinical Laboratory Improvement Amendments (CLIA)-validated immunohistochemistry (IHC) assay. Total duration on treatment trended higher in patients with CKB+ tumors. Tumor specimens from patients with more favorable treatment outcome, i.e. SD/PR are CKB+. Baseline levels of urinary creatine, creatinine and guanidinoacetic acid (GAA) is higher in patients with CKB+ tumors.

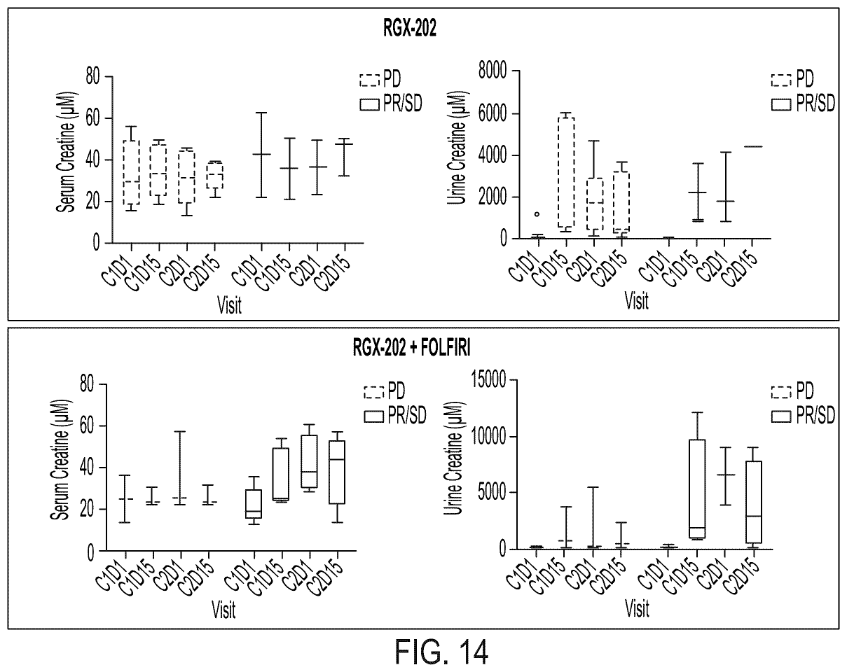

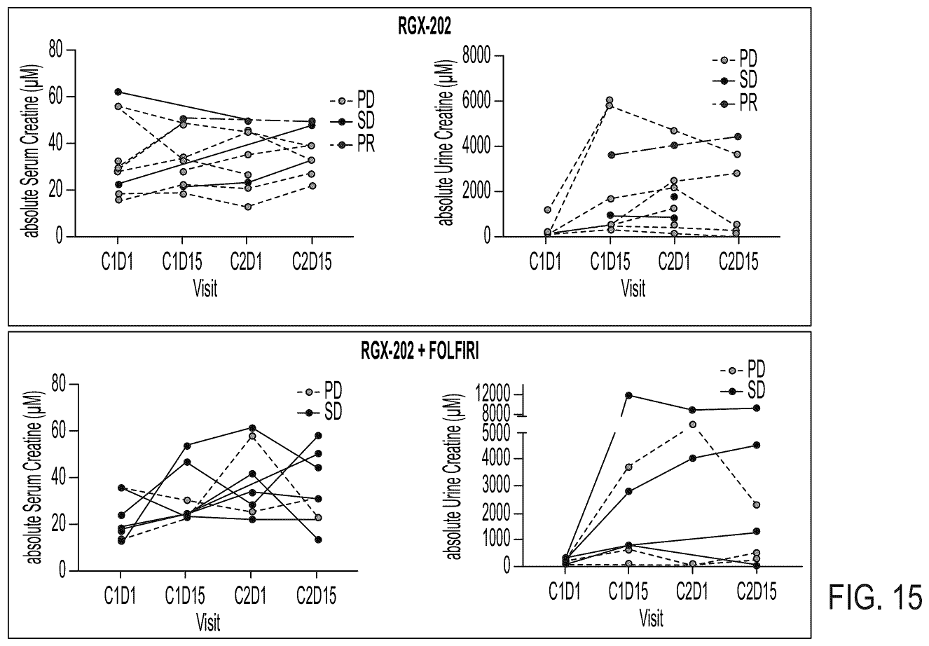

RGX-202-01-mediated changes in creatine metabolism in patients with progressive disease (PD) vs. partial responders/stable disease (PR/SD) patients. Patients with favorable treatment outcome exhibited a more robust and sustained change in pharmacodynamic markers of RGX-202-01 target engagement. After two cycles of treatment, in patients with PR/SD, absolute levels of serum and urine creatine showed an increase over time compared to that observed in patients with PD. This is more evident in patients treated with RGX-202-01+FOLFIRI. The pharmacodynamic effect was more noticeable in urine than in serum (FIG. 14). FIG. 15 shows the subject-level changes in creatine metabolism.

### Example 15: Combination Therapy for the Treatment of KRAS Mutant Cancers with RGX-202-01 and KRAS Inhibitors

Target engagement and clinical validity of RGX-202-01 as a single agent and in combination in combination with infusional 5-fluorouracil (5-FU) and leucovorin plus irinotecan (FOLFIRI), and in combination with FOLFIRI plus bevacizumab is demonstrated by evaluating the effects of SLC6a8 inhibition by RGX-202-01 administration on metabolite levels, such as creatine, and by the levels of CKB expression by immunohistochemistry in tumor tissue. The validity of serum creatinine and creatine levels as pharmacodynamic markers of creatine import inhibition is assessed by evaluating dose-responsiveness of changes in each of the markers after RGX-202-01 administration and combination therapies, such as RGX-202-01+a Kras inhibitor from Table 1. The primary objective during the dose escalation stage is to identify the maximum tolerated dose (MTD), or the maximum tested dose at which multiple dose-limiting toxicities (DLTs) are not observed, of RGX-202-01 as a single agent, and separately, in combination with infusional 5-fluorouracil (5-FU) and leucovorin plus irinotecan (FOLFIRI), and in combination with FOLFIRI plus bevacizumab.

The primary objectives during the expansion stage are the following:
The primary efficacy objective is to estimate the antitumor activity of RGX-202-01 in combination with FOLFIRI plus bevacizumab in patients with previously treated advanced or metastatic colorectal cancer and tumors that express the CKB biomarker.
The primary safety objective is to characterize the safety profile of RGX-202-01 at the MTD or maximum tested dose in combination with FOLFIRI plus bevacizumab. The secondary objectives are to evaluate the pharmacokinetic (PK) profile of RGX-202-01 and potential metabolites in plasma and urine.

The expression of CKB and other relevant markers that include creatine metabolism in tumor specimens are evaluated prior to RGX-202-01 treatment and following treatment are correlated with clinical parameters reflecting anti-cancer activity. Evaluation of pharmacodynamic markers including but not limited to creatine, creatinine, guanidinoacetate (GAA), and lipid levels (including cholesterol and triglycerides) are measured prior to RGX-202-01 treatment, during treatment, and post-treatment.

Multiple doses of orally administered RGX-202-01 with or without FOLFIRI bevacizumab (single agent or combination therapy) is evaluated in patients with advanced gastrointestinal tumors (i.e., locally advanced and unresectable, or metastatic) who have had progressive disease (PD) on available standard systemic therapies or for which there are no standard systemic therapies of relevant clinical impact.

The clinical starting dose for oral administration of RGX-202-01 as a single agent is 1200 mg/day.

The starting dose of RGX-202-01 in combination with FOLFIRI plus bevacizumab is at least one dose level below the last dose of RGX-202-01 that has been evaluated in single agent therapy and which was not considered to be the MTD. The MTD is determined for RGX-202-01 as single agent therapy and in combination with FOLFIRI. The MTD is defined as the highest dose level at which <33% of patients experience Dose Limiting Toxicity (DLT) in the DLT assessment period. Toxicities are assessed using the National Cancer Institute (NCI) Common Terminology Criteria for Adverse Events (CTCAE), version 5.

For all subjects receiving RGX-202-01 combination therapy, the FOLFIRI dose and schedule will be the conventional FOLFIRI dose regimen most commonly used to treat a wide variety of gastrointestinal neoplasms: irinotecan 180 mg/m² intravenously over 90 minutes concurrently with folinic acid 400 mg/m² intravenously over 2 hours, followed by 5-FU 2400 mg/m² intravenous infusion over 46 hours, on Days 1 and 15 of each 28-day cycle.

For all subjects receiving RGX-202-01 combination therapy with bevacizumab, the bevacizumab dose and schedule will be 5 mg/kg on Days 1 and 15 of each 28-day cycle. This is one of the approved bevacizumab dosing regimens for the treatment of colorectal cancer.

In one embodiment, the starting dose regimen of single agent RGX-202-01 is 600 mg BID on Days 1-28 of a 28-day cycle. The starting dose of RGX-202-01 in combination with FOLFIRI will be at least one dose level below the last dose of RGX-202-01 that has been completed as a single agent therapy and was not considered to be the MTD. The starting dose of RGX-202-01 in combination with FOLFIRI plus bevacizumab will be at least one dose level below the last dose of RGX-202-01 that has been evaluated in combination with FOLFIRI and which was not considered to be the MTD.

Patients receive their first dose on Cycle 1, Day 1. A treatment cycle is 28 days in length. All patients receive RGX-202-01 administered orally (PO) on a continuous daily schedule. For all RGX-202-01 patients receiving combination therapy, the FOLFIRI dose and schedule are irinotecan 180 mg/m² intravenously over 90 minutes concurrently with folinic acid 400 mg/m² intravenously over 2 hours, followed by 5-FU 2400 mg/m² intravenous infusion over 46 hours, on Days 1 and 15 of each 28-day cycle, and the bevacizumab dose is 5 mg/kg on Days 1 and 15 of each 28-day cycle.

During treatment, patients are evaluated on an outpatient basis. After discontinuation of therapy, patients are evaluated within 21 days after their last dose of therapy. Follow-up for disease status and survival after discontinuation of therapy can continue for up to 12 months after therapy was started.

Safety is assessed during by documentation of Adverse Events (AE), clinical laboratory tests, physical examination, vital sign measurements, electrocardiograms (ECGs), and Eastern Cooperative Oncology Group (ECOG) performance status (PS). Serial blood samples for PK and pharmacodynamic analyses will be collected from all patients.

Prior to treatment, imaging (computed tomography [CT] scan of chest/abdomen/pelvis or magnetic resonance imaging [MRI], if indicated) and tumor biopsy are conducted. Patients with skin, subcutaneous or lymph node metastases also have tumor evaluations (including measurements, with a ruler) by means of physical examination. Tumor measurements and disease response assessments also are performed at the end of Cycle 2 (approximately 8 weeks after the first study therapy dose), and then approximately every 8 weeks thereafter until development of PD. For patients with evidence of disease control (stable disease or better) at Week 24, tumor measurements and disease response assessments are performed less frequently (approximately every 16 weeks) thereafter.

Tumor measurements and disease response assessments also are performed at the end of therapy. Additionally, measurement of tumor markers (i.e. CEA, CA19-9, CA15-3) is strongly encouraged in situations where a tumor marker is employed in the clinical management of patients with a given malignancy, and the patient has a known elevation of a given marker.

### Example 16: Single Agent Dose Escalation

Following pre-treatment assessment, a subject receives RGX-202-01 by mouth (PO) twice daily (BID) at a total daily dose of 1200 mg/day (600 mg BID) on Days 1-28 of a 28-day cycle. Dose escalation follows a standard 3+3 design.

### Example 17: Combination Dose Escalation and Expansion

Combination therapy dose escalation of RGX-202-01, FOLFIRI, and bevacizumab is initiated once the maximum tolerated dose (MTD) of single agent RGX-202-01 has been identified or at least two cohorts of RGX-202-01 single agent therapy have been completed.

The starting dose of RGX-202-01 in combination with FOLFIRI in subjects is at least one dose level below the last dose of RGX-202-01 that has been evaluated and which was not considered to be the MTD. The starting dose of RGX-202-01 in combination with FOLFIRI plus bevacizumab is at least one dose level below the last dose of RGX-202-01 that has been evaluated in combination with FOLFIRI and which was not considered to be the MTD. A total of approximately 27 patients are treated at the MTD of RGX-202-01 in combination with FOLFIRI plus bevacizumab.

### Study Population

During the dose escalation stage, 24-36 patients with gastrointestinal tumors that are relapsed/refractory to currently available therapies are treated. During the expansion stage, approximately 24 patients among 1 dose expansion cohort with malignant colorectal tumors are treated.

### Example 18: Therapeutics, Dose, and Mode of Administration

### RGX-202-01

RGX-202-01 Capsules, 200 mg or Tablets, 400 mg, will be administered PO. The single agent starting dose regimen of RGX-202-01 (i.e., the dose regimen in Cohort 1) is 1200 mg/day in 600 mg BID divided doses on Days 1-28 every 28 days.

Additional creatine transporter inhibitors that can be used are shown in FIG. 16.

### FOLFIRI

The FOLFIRI chemotherapy regimen, consisting of irinotecan, folinic acid (or leucovorin), and 5-fluorouracil, are administered as follows: irinotecan 180 mg/m² intravenously over 90 minutes concurrently with folinic acid (leucovorin) 400 mg/m² intravenously over 2 hours, followed by 5-FU 2400 mg/m² intravenous infusion over 46 hours, on Days 1 and 15 of each 28-day cycle.

### Bevacizumab

Bevacizumab is a vascular endothelial growth factor inhibitor, and is administered at a dose of 5 mg/kg on Days 1 and 15 of each 28-day cycle.

Efficacy assessments including disease response and progression, duration of response, and survival are evaluated using the following methods:

Tumor tissue (a minimum of 5 and up to 15 unstained slides, or paraffin block) obtained from an archival tissue sample or fresh tissue biopsy is evaluated for CKB biomarker expression and potentially other markers, related to the phosphocreatine transport pathway.

Serum and/or plasma is collected both prior to the first dose of RGX-202-01 and at selected time points after initiation of RGX-202-01 to evaluate levels of creatine, creatinine, and other pharmacodynamic biomarkers.

Plasma concentrations of RGX-202-01 and metabolites is performed on Day 1 and Day 15 of Cycle 1 in patients. Limited plasma concentrations are assessed at other time points to gauge concentrations of RGX-202-01 and identify metabolites at steady-state in these patients. Plasma concentration data over time is used to characterize the pharmacokinetic (PK) disposition of RGX-202-01 and metabolites, to assess any change in the PK properties of RGX-202-01 between initial administration and steady-state, and between cycles of treatment, and relate the PK characteristics of RGX-202-01 to toxicity and anticancer activity. Plasma concentrations are related to the QTc interval and interval changes. For patients enrolled in the dose escalation stage, pooled urine is collected during the 12-hour dosing interval after the first dose on both Days 1 and 15 of Cycle 1 to determine excretion of RGX-202-01 and identify potential metabolites in urine. Pre-treatment spot urine sample is collected prior to Day 1 of Cycle 1.

### Statistical Methods:

Results obtained in connection with RGX-202-01 as a single agent and as a combination therapy are assessed using the following statistical methods. Demographic (e.g., gender, age, race) and baseline characteristics (e.g., Eastern Cooperative Oncology Group (ECOG) performance status, height, weight, and prior therapy) are summarized by RGX-202-01 dose group and tumor type with descriptive statistics.

Treatment-emergent AEs through 30 days after last single agent dose are summarized by MedDRA™ Version 21.0 (or higher) System Organ Class and preferred term. The incidences and percentages of patients experiencing each AE are summarized with descriptive statistics.

AEs are summarized by National Cancer Institute Common Terminology Criteria for Adverse Events (NCI-CTCAE), Version 5 (or higher) grade and by causality (attribution to study treatment). Dose-limiting toxicities, Grade 3-4 AEs, serious adverse events (SAEs), and AEs resulting in dose modification or treatment discontinuation are summarized by preferred term.

Laboratory results are classified according to NCI-CTCAE, Version 5 (or higher). Laboratory results not corresponding to an NCI-CTCAE term will not be graded. Incidences of laboratory abnormalities are summarized with descriptive statistics.

Vital signs and physical examination results are summarized with descriptive statistics.

ORR is defined as the proportion of patients achieving a best response of complete response (CR) or partial response (PR) according to RECIST version 1.1. Duration of response (DoR) is defined as the time from the date measurement criteria are first met for PR or CR to the date measurement criteria are first met for PD. DoCR is defined as the time from the date when the measurement criteria are met for CR to the date measurement criteria are first met for PD. PFS is defined as the time from the date of initiation of study therapy to the date measurement criteria are first met for PD or death from any cause, whichever occurs first. OS is defined as the time from the date of initiation of study therapy to the date of death from any cause. Testing for ORR will be by one-sample binomial tests with a 1-sided type I error rate of 10%. Distributions for PFS, DoR, DoCR, and OS will be estimated by Kaplan-Meier methodology.

Multiple doses of orally administered RGX-202-01 with or without FOLFIRI± bevacizumab (single agent or combination therapy) is evaluated in patients with advanced gastrointestinal tumors (i.e., locally advanced and unresectable, or metastatic) who have had progressive disease (PD) on available standard systemic therapies or for which there are no standard systemic therapies of relevant clinical impact.

Seventeen patients have been treated in the single agent dose escalation stage of the study and 10 patients have been treated in the FOLFIRI dose escalation stage of the study.

TABLE 4
Patient and Disease Characteristics, Study RGX-202-01

| | | Combination |
|---|---|---|
| | Single Agent | with FOLFIRI |
| Patient Characteristics | (N = 17) | (N = 10) |
| Median Age, years (range) | 56 (30, 77) | 63 (47, 76) |
| Gender, n (%) | | |
| Male | 6 (35) | 8 (80) |
| Female | 11 (65) | 2 (20) |
| Cancer Type, n (%) | | |
| Gastric | 0 | 1 (10) |
| Pancreatic | 4 (24) | 0 |
| Colorectal | 13 (76) | 9 (90) |
| Disease Status, n (%) | | |
| Locally Advanced | 2 (12) | 1 (10) |
| Metastatic | 15 (88) | 9 (90) |

Treatment administration and incidences of DLTs by dose for single agent and combination therapy during the dose escalation stage is summarized in Table 4-5. No DLTs among 13 evaluable patients in the single agent cohorts and 9 evaluable patients in the combination with FOLFIRI cohorts had been observed as of the data cut-off date.

TABLE 5
Treatment Regimen, DLTs, and Response, Single Agent and Combination
Therapy Dose Escalation Stage, Study RGX-202-001

| | | Patients Evaluable | Patients with |
|---|---|---|---|
| RGX-202-01 Dose and Regimen | Patients Treated | for DLTs | DLT |
| Single Agent Cohort 1: 600 mg BID | 3 | 3 | 0 |
| Days 1-28 of a 28-day cycle | | | |
| Single Agent Cohort 2: 1200 mg BID | 4 | 3 | 0 |
| Days 1-28 of a 28-day cycle | | | |
| Single Agent Cohort 3: 2400 mg BID | 5 | 4 | 0 |
| Days 1-28 of a 28-day cycle | | | |
| Single Agent Cohort 4: 3600 mg BID | 5 | 3 | 0 |
| Days 1-28 of a 28-day cycle | | | |
| Combination with FOLFIRI Cohort 1: 1800 mg | 3 | 3 | 0 |
| BID Days 1-28 of a 28-day cycle | | | |
| Combination with FOLFIRI Cohort 2: 2400 mg | 4 | 3 | 0 |
| BID Days 1-28 of a 28-day cycle | | | |
| Combination with FOLFIRI Cohort 3: 3000 mg | 3 | 3 | 0 |
| BID Days 1-28 of a 28-day cycle | | | |

Patients who are determined to be eligible, based on Screening assessments, will be enrolled in the study and receive their first dose of study therapy on Cycle 1, Day 1. A treatment cycle is 28 days in length. All patients will receive RGX-202-01 administered orally (PO) on a continuous daily schedule; the dose regimen of RGX-202-01 is dependent on the cohort in which the patient is enrolled. For all RGX-202-01 dosing cohorts receiving combination therapy, the FOLFIRI dose and schedule will be irinotecan 180 mg/m² intravenously over 90 minutes concurrently with folinic acid 400 mg/m² intravenously over 2 hours, followed by 5-FU 2400 mg/m² intravenous infusion over 46 hours, on Days 1 and 15 of each 28-day cycle, and the bevacizumab dose will be 5 mg/kg on Days 1 and 15 of each 28-day cycle.

### Dose Escalation Scheme

Dose escalation will be initiated with single agent RGX-202-01. When at least 2 cohorts of patients treated with single agent RGX-202-01 have been evaluated, dose escalation will commence with RGX-202-01 in combination with fixed dose FOLFIRI. The text below describes dose escalation for single agent therapy, but will also apply to RGX-202-01 in combination therapy.

The starting dose regimen of single agent RGX-202-01 is 600 mg BID on Days 1-28 of a 28-day cycle. The starting dose of RGX-202-01 in combination with FOLFIRI will be at least one dose level below the last dose of RGX-202-01 that has been completed as a single agent therapy and was not considered to be the MTD. The starting dose of RGX-202-01 in combination with FOLFIRI plus bevacizumab will be at least one dose level below the last dose of RGX-202-01 that has been evaluated in combination with FOLFIRI and which was not considered to be the MTD.

### Dosage and Administration of FOLFIRI and Other Combination Therapeutics

All three cytotoxic therapies are administered parenterally on an every 14-day schedule in the FOLFIRI regimen. This regimen is commonly used to treat patients with advanced colorectal and gastric cancer and has been studied in in patients with pancreatic and biliary cancer.

For all RGX-202-01 dosing cohorts receiving combination therapy, the FOLFIRI dose and schedule will be irinotecan 180 mg/m² intravenously over 90 minutes concurrently with folinic acid 400 mg/m² intravenously over 2 hours, followed by 5-FU 2400 mg/m² intravenous infusion over 46 hours, on Days 1 and 15 of each 28-day cycle.

The results obtained in Examples 1-7 were obtained using the following methods and materials.

### Experimental Design

The number of samples for each group was chosen based on knowledge on intra-group variation and expected effect size. Sample sizes for in vitro experiments were chosen based on prior knowledge on intra-group variation. Data was collected until reaching a pre-determined end-point (in vitro assays, PDX trial and clinical sample analysis) or tumor burden exceeded ≥2000 mm³. No data were excluded. Replications were performed as noted in text and figure legends. Replications for all presented experiments were successful. Samples were allocated randomly if possible. Animals were randomized prior to start treatment and mice were sex- and age-matched. No blinding was performed in the in vivo experiments due to cage labeling requirements. Quantification of CKB, cleaved caspase-3 and Ki67 signal by IHC was blinded.

### Animal Strains

All mouse experiments and procedures were approved by the Institutional Animal Care and Use Committees (IACUC) at the New York Blood Center (NYBC), The Rockefeller University and Crown Biosciences. C57BL/6 (JAX stock #000664, RRID: IMSR_JAX:000664), NOD-SCID (JAX stock #001303), Athymic nude (J:NU, JAX stock #007850), NOD scid gamma (NOD.Cg-Prkdcscid Il2rgtm1Wjl/SzJ, JAX stock #005557) and B6129SF1/J (JAX stock #101043) mice were purchased from the Jackson Laboratory. BALB/c mice (stock #028) were purchased from Charles River. SLC6A8 knockout mice were originally obtained from the laboratory of Dr. Skelton (34) and bred in-house. BALB/c nude mice were purchased from Beijing Anikeeper Biotech Co.,Ltd (Beijing, China).

### Primary Tumor Growth Studies

For primary tumor growth experiments, cells (suspended in 50 μl of PBS) were mixed 1:1 with Matrigel (356231, BD Biosciences, Bedford, MA) and subcutaneously injected unilaterally or bilaterally into the lower flank of 6 to 8-week old sex matched mice. Upon detection of tumor volumes reaching the size indicated in each figure, mice were randomly assigned to a drug treatment or a control cohort. RGX-202 was administered through formulated drug chow (Purina 5001, Research Diet, New Brunswick, NJ) at indicated doses or formulated in sterile drinking water for oral gavage. Control cohorts received either regular chow (Purina 5001) or vehicle control. Where xenograft models were tested for biomarker analysis, 10-15 animals were inoculated with the indicated cell lines. Out of these animals, 7-10 mice were assigned to the efficacy portion and were treated for the duration of the experiment. The remaining 3-5 animals were sacrificed when tumors reached ˜500 mm³. Protein and RNA analysis of CKB expression was conducted on the control tumors. Tumor growth measurements were taken using a digital caliper on the days indicated throughout the course of the experiment. Tumor volume was calculated using the formula: volume=(longest diameter)/2×(shortest diameter)². For survival analysis, mice were euthanized when total tumor burden approached IACUC guidelines with a tumor burden exceeding 2000 mm³ in volume. Tumor growth inhibition (TGI) was calculated using the formula TGI (%)=(((ΔCᵢ−ΔC₀)−(ΔTᵢ−ΔT₀))/(ΔCᵢ−ΔC₀))*100%.

### Patient-Derived Xenograft (PDX) Studies

Implantation of PDX's was conducted as described before (22). Briefly, 20-30 mm³ tumor fragments were subcutaneously implanted bilaterally into the flank of 6 to 8-week old age-matched athymic nude mice under general anesthesia with 100 mg/kg ketamine (995-2949-100 mg/mL, Henry Schein Animal Health, Melville, NY) and 10 mg/kg xylazine (X1251, Sigma Aldrich, St. Louis, MO).

### Metastasis Assays

Experiments were conducted by intra-splenic injection of 5×10⁵ luciferase-labeled Lvm3b or CKB CRISPR knock out or CRISPR control Lvm3b cells suspended in 50 L PBS into 6 to 8-week-old NOD SCID mice or NSG mice, respectively that were anesthetized through i.p. injection of ketamine/xylazine solution (100 mg/kg Ketamine, 10 mg/kg xylazine). The day after tumor cell inoculation, mice were randomly assigned to a control or RGX-202 treatment group. Control mice received i.p. injections of 200 μL of PBS and treatment mice received 200 μL of 0.5 M RGX-202 (˜650 mg/kg). Treatment continued daily until the end of the experiment. In the PANC1 experiment, cells grown in D10F complete medium and were pre-treated in vitro with or without RGX-202 at a dose of 10 mM (1.31 mg/mL) RGX-202 for 48 hours prior to injection into the mice. There was no treatment of the mice after the cells were injected. Bioluminescence measurements were conducted once per week for the duration of the experiment. One hundred μL of D-Luciferin (88292, Thermofisher, Waltham, MA, 1 g in 60 mL sterile DPBS) was injected into the venous sinus and the bioluminescence signal over the liver was measured using an IVIS Spectrum In Vivo Imaging System (Perkin Elmer). Photon flux ratio is the ratio of bioluminescence signal at a given time point to the signal on day 0.

### Drug Treatments

5-Fluorouracil (5-FU) (F6627, Millipore Sigma, St. Louis, MO) was administered in sterile 0.9% NaCl by intraperitoneal injections (i.p.) once per week as indicated in the figures. Gemcitabine (G6423, Millipore Sigma, St. Louise, MO) was administered at 100 mg/kg/week i.p. in PBS. Irinotecan (11406-50 mg, Millipore Sigma, St. Louise, MO) was administered at 15 mg/kg/week i.p. in 2.5% DMSO 97.5%, NaCl 0.9%. Leflunomide was formulated in DMSO and administered by daily i.p. injections at 2.5 or 7.5 mg/kg at 0.5 mL/kg. Uridine was formulated in sterile water at 800 mg/kg and administered by daily i.p. injections.

### Cell Culture

UN-KPC-961 cells were obtained from Professor S. K. Batra at Eppley Institute for Research in Cancer (Omaha, Nebraska). COLO 205 (CCL-222), HCT-15 (CCL-225), HT-29 (HTB-38), SW 480 (CCL-228), HCT 116 (CCL-247), HCT-8 (CCL-244), NCI-H508 (CCL-253), HepG2 (HB-8065), Hs746T (HTB-135), CT26 (CRL-2638), LS-174T (CL-88), PANCl (CRL-1469) and NCI-N87 (CRL-5822) cell lines were purchased from ATCC (Baltimore, MD) and maintained in standard conditions following the providers instructions. Lvm3b was generated by in vivo selection from the parental cell line LS-174T (ATCC, Baltimore, MD) (15). UN-KPC-961 cells were maintained in DMEM (11960-044, Gibco, Langley, OK), 7.5% Sodium Bicarbonate (25080-094, Gibco, Langley, OK), 1% Penicillin-Streptomycin (Lonza, 17-745E), 10% Fetal Bovine Sera (F4135, Sigma, St. Louis, MO), 200 mM L-Glutamine (25030081, Gibco, Langley, OK), 1 mM HEPES (15630080, Gibco, Langley, OK) and 50 mg/ml Gentamicin (15750078, Gibco, Langley, OK). MC38 cells were cultured in DMEM, 10% Fetal Bovine Serum and 1 mM HEPES.

### 1×1 PDX Trial (HuTrial)

Details on the models are found in FIG. 9. PDX models were inoculated with a PDX cell suspension or a tumor fragment as follows. Cryogenic vials containing PDX tumor cells were thawed and cells washed in RPMI, counted, and resuspended in cold RPMI at a concentration of 50,000-100,000 viable cells/50 mL. Cell suspensions were mixed with an equal volume of Cultrex ECM. 100 μL of the cell suspension in ECM media was subcutaneously injected into the rear flank of 5 female NOD-SCID mice per model. Alternatively, tumor fragments from stock mice were harvested and used for inoculation into mice. Five 6 to 8-week old female BALB/c nude mice per model was inoculated subcutaneously in the right flank with a primary human tumor fragment (2-3 mm in diameter) for tumor development. Two out of these five mice were randomized into 2 groups (1 mouse per group) when average tumor volume reached 100-150 mm³ and treatment with either control chow (Purina 5001) or RGX-202-formulated chow (in Purina 5001, Research Diet, New Brunswick, NJ) at −400 mg/kg started within 24h after randomization. Randomization was performed based on “Matched distribution” method (StudyDirector™ software, version 3.1.399.19). Tumor growth inhibition on Day 21 (TGI₂₁) was calculated using the formula TGI₂₁=((C₂₁−C₀)/C₂₁)−((T₂₁−T₀)/T₀). The PDX trial was conducted at Crown Biosciences (San Diego, CA and Taicang Jiangsu Province, China).

### Urine and Plasma Creatine Analysis

Six to eight-week old female CD-1 mice were fed with control or RGX-202-01-formulated chow (Purina 5001, Research Diet, New Brunswick, NJ) at 100, 400 and 1200 mg/kg for 10 days. Bleeds were collected in EDTA-coated blood collection tubes (02-669-33, Fisher Scientific) and plasma separated upon centrifugation. Plasma and urine samples were collected from both the control and RGX-202-01-treated cohorts on Day 10 (240 h, 246.5 h, 254.5 h) and Day 11 (264 h) timepoints. Samples were flash frozen and stored at −80° C. until bioanalysis for RGX-202 and creatine levels at Pharmaron (Beijing, China). Mice plasma and urine analysis were conducted as a non-GLP study according to standard operating procedures at Pharmaron (Beijing, China). Briefly, concentration of RGX-202-001 was determined using a surrogate analyte RGX-202-¹³C1¹⁵N₂ by liquid chromatography tandem mass spectrometry (LC-MS/MS). Lower limit of quantification (LLOQ) was determined to be 50.0 ng/mL. The in-life portion of this study was conducted at L2P Research.

### Creatine-(Methyl-d3) Uptake in the Tumors and Heart

Three to four B6129SF1/J male mice harboring UN-KPC-961 tumors that were treated with either control chow or RGX-202-formulated chow at −800 mg/kg for 35 days, received one dose of 1 mg/kg creatine-(methyl-d3) (DLM-1302-0.25, Cambridge Isotope Laboratories, Tewksbury, MA) by i.p. injection. One and a half hours later, the animals were euthanized, the tumors and hearts were extracted, flash frozen in liquid nitrogen and submitted to bioanalysis.

Three to four 6-9-week-old C57BL/6J male wild-type male mice received one dose of RGX-202 at 100, 250 or 500 mg/kg, respectively, in sterile 0.9% NaCl. Seven minutes following injection of RGX-202 or vehicle control, one dose of d3-creatine was administered at 1 mg/kg in sterile 0.9% NaCl by i.p. injection. SLC6A8 knock-out mice received one dose of d3-creatine. One hour later, the mice were anesthetized using 2.5% isoflurane and the animal was perfused with 5-10 mL of DPBS that was injected into the left heart chamber. The heart was then extracted, and flash frozen in liquid nitrogen. Heart samples were stored at −80° C. until being submitted to Seventh Wave Laboratories for analysis. Hearts and tumors were homogenized with a 70/30 methanol/water solution at a 3:1 (v:w) ratio. 20 μL of the homogenate was diluted with 1 mL of internal standard solution (Creatine-d5 in methanol). The samples were centrifuged, and the supernatant was analyzed. A fit-for-purpose, semi-quantitative liquid chromatography with tandem mass spectrometry (LC-MS/MS) method was developed at Seventh Wave Laboratories for the determination of d3-creatine and creatinine in heart and tumor tissue homogenate. Creatinine concentrations were used to normalize the signal of d3-creatine across samples.

### Real-Time PCR Analysis of Tumor Samples

RNA was extracted from 10 mg of flash frozen vehicle or RGX-202-01-treated tumors using Total RNA Purification kit (37500, Norgen Biotek, Thorold, Canada) according to the manufacturer's instructions. RNase-Free DNase I (25710, Norgen Biotek, Thorold, Canada) was used to remove genomic DNA. cDNA synthesis was performed with 600ng of total RNA, using a Verso cDNA synthesis kit (AB-1453/B, Fisher Scientific, Waltham, MA) according to protocol. qPCR was performed using TaqMan Fast Universal PCR Master Mix (2×), no AmpErase UNG (Fisher Scientific, Cat #: 4366073) and TaqMan probes (20×), with 1 μL of 1:5 diluted cDNA.

Predesigned Taqman Gene Expression Assays for CKB, SLC6A8, CKM, CKMT1, CKMT2 and GUSB (Hs00176484 ml, Hs00940515_ml, Hs00176490_ml, Hs00179727_ml, Hs00176502_ml, Hs00939627_ml, Fisher Scientific, Waltham, MA) were used to perform the reactions in a Step One plus Real-time PCR system (Applied Biosystems). GUSB was used as an internal control.

A standard curve for normalization was generated by preparing serial dilutions as follows. Undiluted cDNA from the HCT116 cell line at a stock concentration of 30 ng/μL was used to prepare dilutions of 12.5 ng, 4.17 ng, 1.25 ng, 0.42 ng, 0.125 ng and 0.013 ng. 1:5 diluted cDNA sample of HCT116 cell line was used as a reference that was run in all qPCR plates to account for plate to plate variability. All standards and samples were tested in triplicates or quadruplicates.

### Histology, Immunohistochemistry and Immunofluorescence

Tumors were fixed overnight with 4% PFA (50-980-497, EMS, Hatfield, PA) at 4° C. After two washes with Phosphate Buffer Saline (PBS) (10010023, Gibco), tissue was embedded in paraffin following standard protocols. Tumors were sectioned using a microtome (Leica) and 5 km-thick sections were mounted on Superfrost Plus Microscope Slides (22-037-246, Fisher Scientific). The tissue sections were blocked first for 30 min in Background Blocking reagent (NB306, Innovex). A rabbit monoclonal anti-creatine kinase B type antibody (ab92452, Abcam) was used in 1:400 dilution. The incubation with the primary antibody was done for 5 hours, followed by 60 minutes with biotinylated goat anti-rabbit IgG (PK6101, Vector labs) in 5.75 μg/ml, Blocker D, Streptavidin-HRP and DAB detection kit (Ventana Medical Systems) were used according to the manufacturer's instructions. Slides were counterstained with hematoxylin and coverslipped with Permount (Fisher Scientific). Antibody specificity was determined by staining conducted on HCT116 cells transiently transfected with CKB siRNAs. A tumor microarray (TMA) containing 10 human healthy tissues in duplicates was purchased (CT565861, Origene).

The detection of Ki67 Al 488+ and cleaved caspase-3 CF 594 by immunofluorescence was performed as follows: After 32 min of heat and CCT (Cell Conditioning 1, 950-500, Ventana cat) retrieval, the tissue sections were blocked first for 30 min in Background Blocking reagent (NB306, Innovex). A mouse monoclonal anti-Ki67 antibody (M7240, DAKO) was used in 0.5 μg/mL concentrations. The incubation with the primary antibody was done for 5 hours followed by 5.75 μg/mL biotinylated goat anti-mouse secondary (MOM Kit BMK-2202, Vector Labs). Blocker D, Streptavidin-HRP D (part of DAB Map kit, Ventana Medical Systems), followed by incubation with Tyramide Alexa Fluor 488 (T20922, Invitrogen) prepared according to manufacturer's instruction in 1:150 for 16 min. A rabbit polyclonal anti-cleaved caspase-3 (9661, Cell Signaling) was used in 0.1 mg/ml concentration. The incubation with the primary antibody was done for 5 hours, followed by 60 minutes incubation with biotinylated goat anti-rabbit IgG (PK6101, Vector lab) in 5.75 μg/ml concentration. Blocker D, Streptavidin-HRP and Tyramide-CF594 (92174, Biotium) prepared according to manufacturer instruction in 1:1500 dilution for 16 minutes. After staining, slides were counterstained with DAPI (D9542, Sigma Aldrich, 5 mg/ml) for 10 min and mounted with Mowiol. All stainings were performed at the Molecular Cytology Core Facility of Memorial Sloan Kettering Cancer Center, using Discovery XT processor (Ventana Medical System, Roche—Indianapolis, IN)

### Analysis of Tumor Samples

Six to eight tumors per xenograft model was assigned a score of β-3 based on intensity of CKB staining. Score 0 is interpreted as negative for protein expression while scores 1, 2, and 3 are interpreted as positive staining for each core with 3 being maximal intensity. For each positive sample, the area (% of tumor) corresponding to each intensity staining was recorded to allow for calculation of percentage tumor positivity (Tumor Proportion Score). A weighted overall staining score (H-score) was calculated as (percentage area of 1+staining ×1)+(percentage area of 2+staining ×2)+(percentage area of 3+staining ×3). Tumor sections were excluded from the analysis if that section was predominantly necrotic. In these cases, new tumor sections were stained to reach a minimum of n=3-4 sections per tumor, 3 tumors per cohort.

### Image Quantification

Quantification of the number of Ki67 positive cells, and percentage of CC3 per area was performed using Image J (version 1.50i). Three tumors per group were selected and 3 to five fields per tumor were chosen for quantification.

### Hypoxia Cell Growth Assay

Lvm3b cells were seeded at 300,000 cells/well in a 6 well plate. After 24 hours incubation under normoxia, the cells were cultured under hypoxia (0.5% oxygen, 5% C02, 37° C.) for 96 hours in the presence of RGX-202, creatine (Cr) (Sigma-Aldrich #C3630) or phosphocreatine (PCr) (Sigma-Aldrich #237911) at 10 μM. The media was replaced and incubation continued up to 120 hours, when cells were counted.

### Metabolite Extraction and Liquid Chromatography

Lvm3b cells were plated at 3×10⁵ cells/well in triplicates in RPMI1640 in the presence of dialyzed FBS, 2 mM glutamine and 6 mM glucose and allowed to adhere to the plate for 24 hr. Cells are treated with either control or 10 mM RGX-202 for 24 hours in 0.5% 02. Cells were washed with ice cold 0.9% NaCl and harvested in ice cold 80:20 LC-MS methanol:water (v/v). Samples were vortexed vigorously and centrifuged at 20,000 g at maximum speed at 4° C. for 10 min. The supernatant was transferred to new tubes. Samples were then dried to completion using a nitrogen dryer. All samples were reconstituted in 30 μl 2:1:1 LC-MS water:methanol:acetonitrile. The injection volume for polar metabolite analysis was 5 μL. Metabolite extraction and subsequent Liquid-Chromatography coupled to High-Resolution Mass Spectrometry (LC-HRMS) for polar metabolites of cells was carried out using a Q Exactive Plus.

### Liquid Chromatography

A ZIC-pHILIC 150×2.1 mm (5 μm particle size) column (EMD Millipore) was employed on a Vanquish Horizon UHPLC system for compound separation at 40° C. The autosampler tray was held at 4° C. Mobile phase A is water with 20 mM Ammonium Carbonate, 0.1% Ammonium Hydroxide, pH 9.3, and mobile phase B is 100% Acetonitrile. The gradient is linear as follows: 0 min, 90% B; 22 min, 40% B; 24 min, 40% B; 24.1 min, 90% B; 30 min, 90% B. The follow rate was 0.15 ml/min. All solvents are LC-MS grade and purchased from Fisher Scientific.

### Mass Spectrometry

The Q Exactive Plus MS (Thermo Scientific) is equipped with a heated electrospray ionization probe (HESI) and the relevant parameters include: heated capillary, 250° C.; HESI probe, 350° C.; sheath gas, 40; auxiliary gas, 15; sweep gas, 0; spray voltage, 3.0 kV. A full scan ranged from 55 to 825 (m z) was used. The resolution was set at 70,000. The maximum injection time was 80 milliseconds (ms). Automated gain control (AGC) was targeted at 1×10⁶ ions.

Maximum injection time was 20 msec. Raw data collected from LC-Q Exactive Plus MS was processed on Skyline (available on the world wide web at https://<skyline.ms/project/home/software/Skyline/begin.view>) using a five ppm mass tolerance and an input file of m z and detected retention time of metabolites from an in-house library of chemical standards. The output file including detected m z and relative intensities in different samples was obtained after data processing. Quantitation and statistics were calculated using Microsoft Excel, GraphPad Prism 8.1, and Rstudio 1.0.143.

### Clinical Specimen Analysis

Serum specimens were drawn from patients on Cycle 1 Day 15 at 0 hours (h), 0.5h, 1, 1.5h, 2h, 4h, 6h, 8h, 10h, 12h after administration of RGX-202-01 and were analyzed for RGX-202 using an analytically validated LC-MS/MS method at Syneos Health. Urine specimens were collected on Cycle 1 Day 15 at 0, 4 and 12 h after administration of RGX-202; average values are presented in the figure. Serum sample for creatine analysis was collected at 12h post-administration. Creatine levels were analyzed by Q2 solutions at contract research laboratories.

### Patient Details

In the RGX-202-01 Phase 1 a/b human study, adult patients of both sexes over the age of 18 years were enrolled. Data from 13 patients were analyzed. The study is currently accruing and ongoing.

### Clinical Study Design

This Phase 1 a/b study is open-label, multi-center and single-arm, whose primary objective is to determine the maximum-tolerated dose, or maximum tested dose at which multiple dose-limiting toxicities (DLTs) are not observed, of RGX-202-01. Inclusion and exclusion criteria are stipulated, requiring all patients to have a pathologic confirmation of a locally advanced or metastatic solid tumor or lymphoma that has been deemed refractory to standard therapies. Patients may not have any other active malignancy that could confound the study endpoints. Patients must not have a history of pancreatitis, active Hepatitis B or C, or any illness/social situation in the opinion of the treating investigator that would limit compliance with the study requirements. Patients are not allowed to be treated with any other anti-neoplastic therapies while on study. Typical Phase 1 study parameters are required for performance status, hematologic and other organ function measurements. Patients are required to use acceptable contraceptive methods while on study and for a specified time period thereafter. Concomitant medications are restricted only if they pose a clinical risk of drug-drug interactions. Treatment for any condition with corticosteroids is not allowed unless at doses less than 10 mg daily prednisone equivalents. Patients were treated with RGX-202 at 600 mg, 1200 mg, 2400 mg or 3600 mg twice/day continuous dosing, according to the patient cohort to which they were enrolled. Patients continued treatment with RGX-202-01 until treatment intolerance or progression of disease. The primary endpoint is incidence of DLTs, which are evaluated by the medical monitor in collaboration with all treating clinical investigators. Secondary endpoints include pharmacokinetic measurements of RGX-202 and its metabolites in plasma and urine. Exploratory endpoints include measuring of serum and urine levels of creatine. Ultimately, efficacy endpoints will be obtained on a large sample size of patients in disease-specific expansion cohorts. Dose-limiting toxicities are defined as any of the following toxicities occurring during the first 4 weeks of treatment that are not clearly related to another cause (i.e., disease progression): any grade ≥3 non-hematologic AE, with the exceptions of Grade 3 nausea, vomiting, diarrhea, constipation, fever, fatigue, or skin rash in which there has been suboptimal prophylaxis and management that resolves to Grade % 2 within 72 hours; grade 4 thrombocytopenia, or Grade 3 thrombocytopenia with Grade ≥1 bleeding or requirement for platelet transfusion; grade 4 neutropenia; grade ≥3 febrile neutropenia; grade ≥3 transaminase (AST/ALT) elevation; any toxicity resulting in ≥25% held/skipped doses during the cycle; any other significant toxicity considered by the Investigator Sponsor's medical representatives to be dose-limiting.

### Statistical Analysis

Significance of tumor growth curve comparisons was carried out using two-sided t tests. Metastasis assays were analyzed using the non-parametric Mann-Whitney test. The Mantel-Cox log-rank test was used for statistical comparisons in survival analyses. Statistical analysis of creatine and RGX-202 AUC correlations in human patients and mice were carried out using simple linear regression with Prism 8. Statistical comparisons of cleaved caspase-3 and Ki67 signal, in vitro and in vivo d3-creatine analysis, qPCR and IHC quantification were carried out using two-sided t tests. T-test was used to compare metabolite abundance with Bonferroni's multiple test correction where indicated. Throughout all figures: *p<0.05, **p<0.01, and ***p<0.001, ****p<0.0001. Significance was concluded at p<0.05.

### Example 18: Tumor Growth Inhibition Achieved Using RGX-202 in Combination with a Low-Dose KRAS Inhibitor

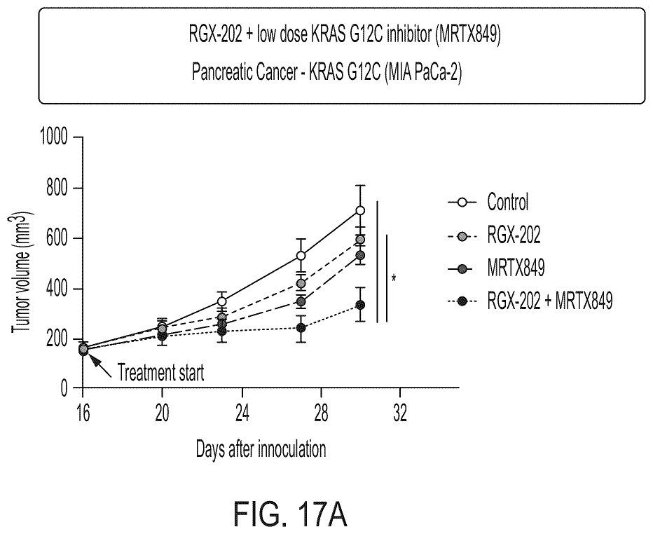

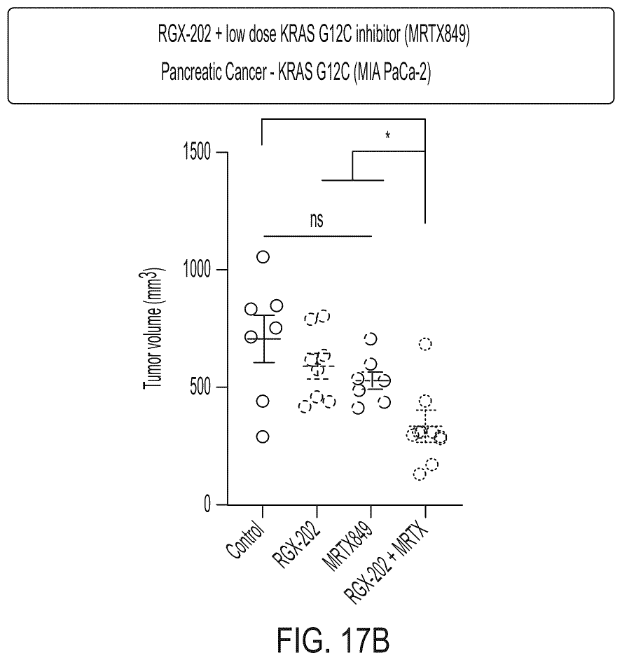

An experiment was undertaken to evaluate suppression of tumor growth in a mouse KRAS G12C RAS mutant synergeneic pancreatic cancer model (MIA PaCA-2) using RGX-202 and MRTX849 (Adagrasib) alone or in combination. Mice were treated with RGX-202 (800 mg/kg), MRTX849 (3 mg/kg starting at day 16 and then 10 mg/kg starting at day 23), control, or a combination of RGX-202 and MRTX849 (n=6-7/cohort). Growth of the xenografts in the mice was measured over time. Administration of RGX-202 in combination with MRTX849 was the most effective treatment for inhibiting tumor growth (FIGS. 17A and 17B).

### Example 19: RGX-202 was Effective in Inhibiting Growth of Non-Small Cell Lung Cancer (NSCLC) Xenografts

Experiments were undertaken to evaluate inhibition of non-small cell lung cancer (NSCLC) xenografts in mice using RGX-202.

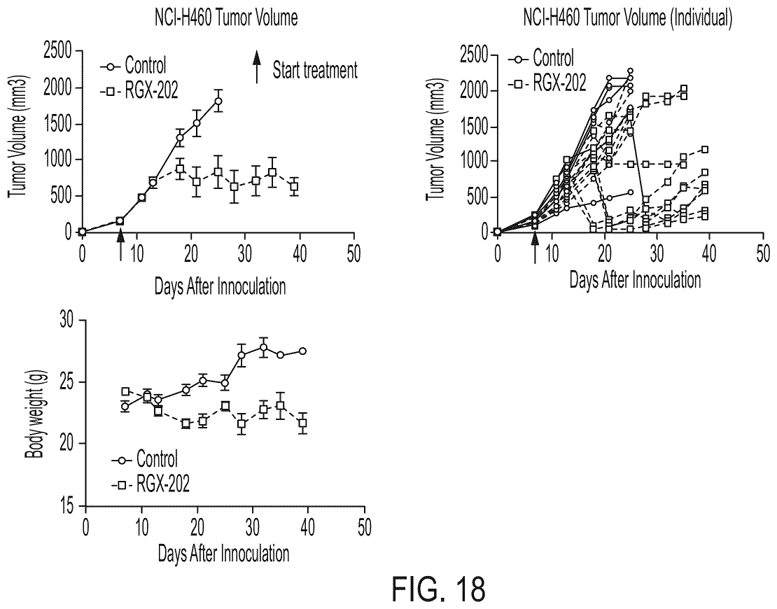

Administration of RGX-202 led to tumor growth inhibition in NCL-H460 KRASQ61H xenografts in mice (FIG. 18). 4×10⁶ NCI-H460 cells were injected in 6-8 week old female Nude mice. Treatment with RGX-202-formulated chow started when tumors reached ˜200 mm³. NCI-H460 is a non-small cell lung cancer cell line that harbors the KRAS Q61H mutation. The mice were administered RGX-202 at a dose of 800 mg/kg.

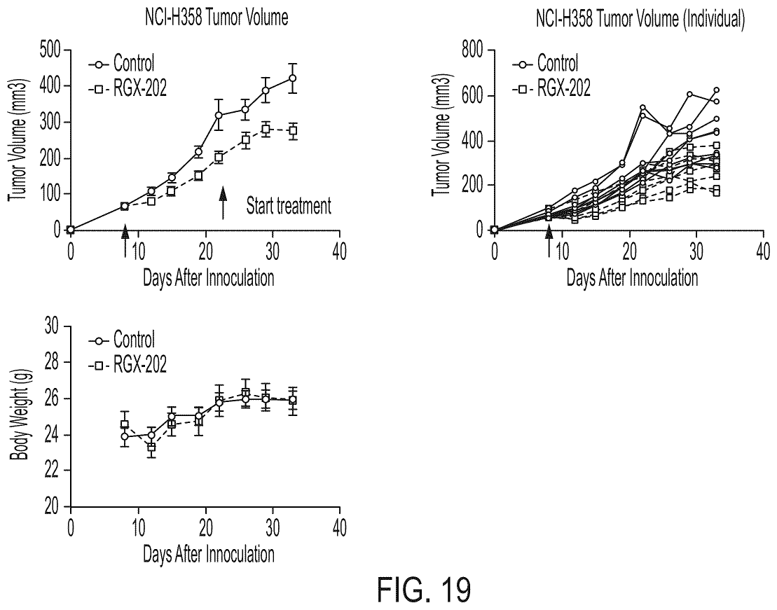

Administration of RGX-202 also led to tumor growth inhibition in NCL-H358 KRASG12C xenografts in mice (FIG. 19). 5×10⁶ NCI-H358 cells were injected in 6-8 week old female Nude mice. Treatment with RGX-202-formulated chow started when tumors reached ˜80 mm³. H358 is a non-small cell lung cancer cell line that harbors the KRAS G12C mutation. The mice were administered RGX-202 at a dose of 800 mg/kg.

### OTHER EMBODIMENTS

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. A method of inhibiting cancer cell proliferation or survival, the method comprising contacting a cancer cell with a creatine transporter inhibitor and a KRAS inhibitor, thereby inhibiting cancer cell proliferation or survival.

2. The method of claim 1, wherein the creatine transporter is SLC6A8 or is selected from those listed in Table 2.

3. The method of claim 1, wherein the creatine transporter inhibitor is selected from the group consisting of β-Guanidinopropionic acid, N-methylamidino-N-methylglycine, 1-carboxymethyl-2-imino-hexahydropyrimidine (cyclocreatine), DL-alpha-guanidinopropionic acid, N-methyl-N-amidino-beta-alanine, N-ethyl-N-amidinoglycine, DL-alpha-guanidinobutyric acid, DL-beta-guanidinobutyric acid, gamma-guanidinobutyric acid, and guanidinoacetic acid, or a pharmaceutically acceptable salt thereof.

4. The method of claim 3, wherein the creatine transporter inhibitor is (β-Guanidinoproprionic acid (β-GPA) or a pharmaceutically acceptable salt thereof.

5. The method of claim 1, further comprising contacting the cancer cell with an EGFR inhibitor.

6. The method of claim 1, wherein a KRAS inhibitor is selected from the group consisting of Sotorasib, Adagrasib, MRTX1133, RMC-6291, and RMC-6236.

7. The method of claim 1, wherein the cancer cell is contacted with Adagrasib and β-GPA.

8. The method of claim 1, wherein the cancer cell is a colorectal cancer cell.

9. A method of inhibiting cancer cell proliferation or survival, the method comprising contacting a cancer cell with β-GPA and Adagrasib, thereby inhibiting cancer cell proliferation or survival.

10. The method of claim 9 further comprising contacting the cancer cell with an EGFR inhibitor.

11. A method of inhibiting cancer cell proliferation or survival, the method comprising contacting a cancer cell with a creatine transporter inhibitor, FOLFIRI comprising leucovorin (folinic acid-FOL), 5-fluorouracil (F), and irinotecan hydrochloride (IRI), and/or bevacizumab, thereby inhibiting cancer cell proliferation or survival.

12. The method of claim 11, wherein the cancer cell is contacted with β-GPA, FOLFIRI, and bevacizumab.

13. The method of claim 1, wherein the method further comprises contacting the cell with one or more additional chemotherapeutic agents selected from the group consisting of atovaquone, brequinar sodium, teriflunomide, BAY-2402234, AG-636, leucovorin, 5-fluorouracil, irinotecan, and oxaliplatin.

14. A method of treating cancer in a subject, the method comprising administering to the subject a creatine transporter inhibitor and a KRAS inhibitor.

15. The method of claim 14, wherein the creatine transporter inhibitor is selected from the group consisting of β-Guanidinopropionic acid, N-methylamidino-N-methylglycine, 1-carboxymethyl-2-imino-hexahydropyrimidine (cyclocreatine), DL-alpha-guanidinopropionic acid, N-methyl-N-amidino-beta-alanine, N-ethyl-N-amidinoglycine, DL-alpha-guanidinobutyric acid, DL-beta-guanidinobutyric acid, gamma-guanidinobutyric acid, and guanidinoacetic acid;
and the KRAS inhibitor is selected from the group consisting of Sotorasib, Adagrasib, MRTX1133, RMC-6291, and RMC-6236.

16. A method of treating a subject having a cancer, the method comprising administering to the subject β-GPA, 5-fluorouracil.

17. A method of treating cancer in a subject, the method comprising administering to the subject a creatine transporter inhibitor, FOLFIRI comprising leucovorin (folinic acid-FOL), 5-fluorouracil (5-FU), and irinotecan hydrochloride (IRI), and/or bevacizumab.

18. A method of treating cancer in a subject, the method comprising administering to the subject a creatine transporter inhibitor, and FOLFOX comprising leucovorin (folinic acid-FOL), 5-fluorouracil (5-FU), and oxaliplatin (OX).

19. A method of treating a selected subject, the method comprising administering to the subject a creatine transporter inhibitor and a KRAS inhibitor, wherein the subject is selected as having a cancer characterized by increased levels of creatine and/or creatine kinase B expression and/or as having a cancer comprising a KRAS, HRAS, or NRAS mutation relative to a control cancer.

20. The method of claim 19, wherein the creatine transporter inhibitor is selected from the group consisting of β-Guanidinopropionic acid, N-methylamidino-N-methylglycine, 1-carboxymethyl-2-imino-hexahydropyrimidine (cyclocreatine), DL-alpha-guanidinopropionic acid, N-methyl-N-amidino-beta-alanine, N-ethyl-N-amidinoglycine, DL-alpha-guanidinobutyric acid, DL-beta-guanidinobutyric acid, gamma-guanidinobutyric acid, and guanidinoacetic acid.

21. The method of claim 19, further comprising administering to the subject an EGFR inhibitor.

22. A method for treating a subject having cancer, the method comprising administering to the subject β-GPA or a pharmaceutically acceptable salt thereof and FOLinic acid-Fluorouracil-IRInotecan regimen (FOLFIRI), wherein β-GPA or its salt form is administered two times daily at a dosage of 1000 mg-3600 mg.


SEQUENCE LISTING (10 sequences)

SEQ ID NO: 1 - protein, 381 aa, Homo sapiens
MPFSNSHNALKLRFPAEDEFPDLSAHNNHMAKVLTPELYAELRAKSTPSGFTLDDVIQTGVDNPGHPYIMTVGCVAGDEESYEVFKDLFDPIIEDRHGGYKPSDEHKTDLNPDNLQGGDDLDPNYVLSSRVRTGRSIRGFCLPPHCSRGERRAIEKLAVEALSSLDGDLAGRYYALKSMTEAEQQQLIDDHFLFDKPVSPLLLASGMARDWPDARGIWHNDNKTFLVWVNEEDHLRVISMQKGGNMKEVFTRFCTGLTQIETLFKSKDYEFMWNPHLGYILTCPSNLGTGLRAGVHIKLPNLGKHEKFSEVLKRLRLQKRGTGGVDTAAVGGVFDVSNADRLGFSEVELVQMVVDGVKLLIEMEQRLEQGQAIDDLMPAQK

SEQ ID NO: 2 - protein, 189 aa, Homo sapiens
MTEYKLVVVGAGGVGKSALTIQLIQNHFVDEYDPTIEDSYRKQVVIDGETCLLDILDTAGQEEYSAMRDQYMRTGEGFLCVFAINNTKSFEDIHQYREQIKRVKDSDDVPMVLVGNKCDLAARTVESRQAQDLARSYGIPYIETSAKTRQGVEDAFYTLVREIRQHKLRKLNPPDESGPGCMSCKCVLS

SEQ ID NO: 3 - protein, 189 aa, Homo sapiens [sequence omitted]

SEQ ID NO: 4 - protein, 188 aa, Homo sapiens [sequence omitted]

SEQ ID NO: 5 - protein, 75 aa, Homo sapiens
MTEYKLVVVGAGGVGKSALTIQLIQNHFVDEYDPTIEGVDDAFYTLVREIRKHKEKMSKDGKKKKKKSKTKCVIM

SEQ ID NO: 6 - protein, 189 aa, Homo sapiens
MTEYKLVVVGAGGVGKSALTIQLIQNHFVDEYDPTIEDSYRKQVVIDGETCLLDILDTAGQEEYSAMRDQYMRTGEGFLCVFAINNSKSFADINLYREQIKRVKDSDDVPMVLVGNKCDLPTRTVDTKQAHELAKSYGIPFIETSAKTRQGVEDAFYTLVREIRQYRMKKLNSSDDGTQGCMGLPCVVM

SEQ ID NO: 7 - protein, 593 aa, Homo sapiens [sequence omitted]

SEQ ID NO: 8 - protein, 635 aa, Homo sapiens
MAKKSAENGIYSVSGDEKKGPLIAPGPDGAPAKGDGPVGLGTPGGRLAVPPRETWTRQMDFIMSCVGFAVGLGNVWRFPYLCYKNGGGVFLIPYVLIALVGGIPIFFLEISLGQFMKAGSINVWNICPLFKGLGYASMVIVFYCNTYYIMVLAWGFYYLVKSFTTTLPWATCGHTWNTPDCVEIFRHEDCANASLANLTCDQLADRRSPVIEFWENKVLRLSGGLEVPGALNWEVTLCLLACWVLVYFCVWKGVKSTGKIVYFTATFPYVVLVVLLVRGVLLPGALDGIIYYLKPDWSKLGSPQVWIDAGTQIFFSYAIGLGALTALGSYNRFNNNCYKDAIILALINSGTSFFAGFVVFSILGFMAAEQGVHISKVAESGPGLAFIAYPRAVTLMPVAPLWAALFFFMLLLLGLDSQFVGVEGFITGLLDLLPASYYFRFQREISVALCCALCFVIDLSMVTDGGMYVFQLFDYYSASGTTLLWQAFWECVVVAWVYGADRFMDDIACMIGYRPCPWMKWCWSFFTPLVCMGIFIFNVVYYEPLVYNNTYVYPWWGEAMGWAFALSSMLCVPLHLLGCLLRAKGTMAERWQHLTQPIWGLHHLEYRAQDADVRGLTTLTPVSESSKVVVVESVM

SEQ ID NO: 9 - protein, 625 aa, Homo sapiens
MAKKSAENGIYSVSGDEKKGPLIAPGPDGAPAKGDGPVGLGTPGGRLAVPPRETWTRQMDFIMSCVGFAVGLGNVWRFPYLCYKNGGGVFLIPYVLIALVGGIPIFFLEISLGQFMKAGSINVWNICPLFKGLGYASMVIVFYCNTYYIMVLAWGFYYLVKSFTTTLPWATCGHTWNTPDCVEIFRHEDCANASLANLTCDQLADRRSPVIEFWENKVLRLSGGLEVPGALNWEVTLCLLACWVLVYFCVWKGVKSTGKIVYFTATFPYVVLVVLLVRGVLLPGALDGIIYYLKPDWSKLGSPQVWIDAGTQIFFSYAIGLGALTALGSYNRFNNNCYNGTSFFAGFVVFSILGFMAAEQGVHISKVAESGPGLAFIAYPRAVTLMPVAPLWAALFFFMLLLLGLDSQFVGVEGFITGLLDLLPASYYFRFQREISVALCCALCFVIDLSMVTDGGMYVFQLFDYYSASGTTLLWQAFWECVVVAWVYGADRFMDDIACMIGYRPCPWMKWCWSFFTPLVCMGIFIFNVVYYEPLVYNNTYVYPWWGEAMGWAFALSSMLCVPLHLLGCLLRAKGTMAERWQHLTQPIWGLHHLEYRAQDADVRGLTTLTPVSESSKVVVVESVM

SEQ ID NO: 10 - protein, 520 aa, Homo sapiens
MKAGSINVWNICPLFKGLGYASMVIVFYCNTYYIMVLAWGFYYLVKSFTTTLPWATCGHTWNTPDCVEIFRHEDCANASLANLTCDQLADRRSPVIEFWENKVLRLSGGLEVPGALNWEVTLCLLACWVLVYFCVWKGVKSTGKIVYFTATFPYVVLVVLLVRGVLLPGALDGIIYYLKPDWSKLGSPQVWIDAGTQIFFSYAIGLGALTALGSYNRFNNNCYKDAIILALINSGTSFFAGFVVFSILGFMAAEQGVHISKVAESGPGLAFIAYPRAVTLMPVAPLWAALFFFMLLLLGLDSQFVGVEGFITGLLDLLPASYYFRFQREISVALCCALCFVIDLSMVTDGGMYVFQLFDYYSASGTTLLWQAFWECVVVAWVYGADRFMDDIACMIGYRPCPWMKWCWSFFTPLVCMGIFIFNVVYYEPLVYNNTYVYPWWGEAMGWAFALSSMLCVPLHLLGCLLRAKGTMAERWQHLTQPIWGLHHLEYRAQDADVRGLTTLTPVSESSKVVVVESVM